(19) United States
(12) Patent Application Publication
(10) Pub. No.: US 2025/0069699 A1
(43) Pub. Date: Feb. 27, 2025

(54) Methods and Systems for Discovery of Embedded Target Genes in Biosynthetic Gene Clusters

(71) Applicant: LifeMine Therapeutics, Inc., Cambridge, MA (US)

(72) Inventors: Michalis HADJITHOMAS, Cambridge, MA (US); Stephen Andrew WYKA, Cambridge, MA (US); Jinwoo KIM, Acton, MA (US); Yu-Cheng LIN, Taipei City (TW); Iain James MCFADYEN, Cambridge, MA (US); Gregory L. VERDINE, Newton, MA (US)

(21) Appl. No.: 18/654,873

(22) Filed: May 3, 2024

Related U.S. Application Data
(63) Continuation of application No. PCT/US2022/049040, filed on Nov. 4, 2022.
(60) Provisional application No. 63/278,065, filed on Nov. 10, 2021.
(60) Provisional application No. 63/263,638, filed on Nov. 5, 2021.

(51) Int. Cl. G16B 30/10; G16B 10/00; G16B 40/20

(52) U.S. Cl. CPC G16B 30/10; G16B 10/00; G16B 40/20

(57) ABSTRACT

The present disclosure relates to computer-based methods and systems for identifying genes associated with biosynthetic gene clusters (BGCs), including embedded target genes (ETaGs) that are homologs of potential therapeutic targets, using comparative genomics techniques and machine learning models.

### CROSS-REFERENCE TO RELATED APPLICATIONS

This application is a continuation of International Application No. PCT/US22/049040, which designated the United States and was filed on Nov. 4, 2022, published in English, which claims the priority benefit of U.S. Provisional Patent Application Ser. No. 63/263,638, filed Nov. 5, 2021, and the priority benefit of U.S. Provisional Patent Application Ser. No. 63/278,065, filed Nov. 10, 2021, the contents of each of which are incorporated herein by reference in their entirety.

### FIELD

The present disclosure relates generally to methods and systems for identifying genes associated with gene clusters (e.g., biosynthetic gene clusters), and applications thereof including methods of determining the boundaries of gene clusters (e.g., the boundaries of biosynthetic gene clusters), methods for identifying therapeutic targets, and methods for drug discovery.

### BACKGROUND

Microbes produce a wide variety of small molecule compounds, known as secondary metabolites or natural products, which have diverse chemical structures and functions. Some secondary metabolites allow microbes to survive adverse environments, while others serve as weapons of inter- and intra-species competition. See, e.g., Piel, J. Nat. Prod. Rep., 26:338-362, 2009. Many human medicines (including, e.g., antibacterial agents, antitumor agents, and insecticides) have been derived from secondary metabolites. See, e.g., Newman D. J. and Cragg G. M. J. Nat. Prod., 79: 629-661, 2016.

Microbes synthesize secondary metabolites using enzyme proteins encoded by clusters of co-located genes called biosynthetic gene clusters (BGCs). Evidence is emerging that some microbial biosynthetic gene clusters contain genes that appear not to be involved in synthesis of the relevant biosynthetic products produced by the enzymes encoded by the clusters. In some cases, such non-biosynthetic genes have been described as “self-protective” because they encode proteins that apparently can render the host organism resistant to the relevant biosynthetic product. For example, in some cases, non-biosynthetic genes encoding transporters of the biosynthetic products, detoxification enzymes that act on the biosynthetic products, or resistant variants of proteins whose activities are targeted by the biosynthetic products, have been reported. See, for example, Cimermancic, et al., Cell 158: 412, 2014; Keller, Nat. Chem. Biol. 11:671, 2015. Researchers have proposed that identification of such genes, and determination of their functions, could be useful in determining the role of the biosynthetic products synthesized by the enzymes of the clusters. See, for example, Yeh, et al., ACS Chem. Biol. 11:2275, 2016; Tang, et al., ACS Chem. Biol. 10: 2841, 2015; Regueira, et al., Appl, Environ. Microbiol. 77: 3035, 2011; Kennedy, et al., Science 284: 1368, 1999; Lowther, et al., Proc. Natl. Acad. Sci. USA 95: 12153, 1998; Abe, et al., Mol. Genet. Genomics 268: 130, 2002. United States Patent Application Publication No. 2020/0211673 A1 provides insights that certain non-biosynthetic genes present in biosynthetic gene clusters, or present in close proximity to the biosynthetic genes of the clusters (particularly in eukaryotic, e.g., fungal, biosynthetic gene clusters as contrasted with bacterial biosynthetic gene clusters) may represent homologs of human genes that are targets of therapeutic interest. Such non-biosynthetic genes are referred to as “embedded target genes” or “ETaGs.”

Traditionally, secondary metabolites have been identified from microbial cultures and screened for therapeutic activities against human targets of interest. However, the vast majority of microbes are not culturable, and even BGCs in culturable microbes can remain transcriptionally silent under laboratory conditions. Recent developments in nucleic acid and protein sequencing technologies and bioinformatics pipelines have enabled rapid identification of a large number of BGCs from environmental microbes without having to culture the microbes and test the bioactivity of the BGCs. See, e.g., Palazzotto E. and Weber T., Curr. Opin. Microbiol., 45:109-116, 2018. However, it remains a challenge to precisely define the genomic boundaries of BGCs using purely computational methods. There are also no computational pipelines available to identify embedded genes in BGCs that confer self-protection against the secondary metabolites produced by the BGCs.

### SUMMARY

Disclosed herein are exemplary methods and systems for identifying genes associated with a biosynthetic gene cluster (BGC) in a target genome. The disclosed methods and systems provide genomic database search and analysis tools that may be used for identifying putative embedded target gene sequences (pETaGs) in one or more target genomes that are homologs of a known ETaG sequence in a first genome. Also disclosed are methods and systems for evaluating the likelihood that a given pETaG is an actual ETaG (e.g., a resistance gene against a secondary metabolite produced by the BGC). The disclosed methods and systems may also be used for determining the boundaries of a BGC in the one or more target genomes, or for assisting in the identification of a small molecule modulator of a target gene of interest.

Disclosed herein are computer-implemented methods for identifying an embedded target gene (ETaG) comprising: specifying one or more query sequences, or proxies thereof; selecting one or more target genomes; performing a search of the one or more target genomes using the one or more query sequences, or proxies thereof, to identify putative embedded target gene (pETaG) sequences that are homologs of the one or more query sequences based on a comparison of one or more homologous sequence-based metrics for candidate pETaGs to one or more predetermined homologous sequence-based metric thresholds; and determining whether or not a given pETaG is an ETaG based on a comparative genomics analysis of a plurality of genomes.

In some embodiments, the comparative genomics analysis comprises generating a comparative genomics heatmap based on the plurality of genomes. In some embodiments, the plurality of genomes comprises a plurality of positive genomes and a plurality of negative genomes. In some embodiments, the comparative genomics analysis comprises determining a phylogenetic feature, a co-occurrence feature, a co-evolution feature, or any combination thereof, for the given pETaG based on the plurality of genomes.

In some embodiments, the comparative genomics analysis comprises an analysis of an input data set comprising phylogenetic features, co-occurrence features, co-evolution features, comparative genomics heatmaps, data derived from comparative genomics heatmaps, or any combination thereof, for a pETaG using a machine learning model or an empirical algorithm to predict a probability that the pETaG is an ETaG.

In some embodiments, the computer-implemented method further comprises determining that an identified pETaG is related to a resistance mechanism based on a determination of a copy number for the identified pETaG. In some embodiments, the computer-implemented method further comprises determining that a pETaG is related to a resistance mechanisms based on a determination of a copy number difference between positive genomes that comprise the pETaG and negative genomes that to not comprise the pETaG.

In some embodiments, the one or more query sequences, or proxies thereof, comprise one or more protein sequences, one or more nucleic acid sequences, one or more Universal Protein Resource (Uniprot) identification numbers, one or more profile Hidden Markov Models (pHMMs), a specified set of protein sequence domains, or any combination thereof. In some embodiments, the one or more query sequences, or proxies thereof, are selected from a bacterial genome, an archaebacterial genome, a fungal genome, a plant genome, an animal genome, a human genome, or any combination thereof.

In some embodiments, the one or more target genomes are selected from a bacterial genome, a fungal genome, a plant genome, or any combination thereof. In some embodiments, two or more target genomes are selected based on a pairwise similarity score, a pairwise phylogenetic distance, or any combination thereof.

In some embodiments, the computer-implemented method further comprises filtering the two or more selected target genomes to retain only those target genomes for which: (i) the pairwise similarity score is greater than a specified pairwise similarity threshold, or (ii) the pairwise phylogenetic distance is less than a specified phylogenetic distance threshold.

In some embodiments, the computer-implemented method further comprises clustering the retained target genomes into sets using a clustering algorithm, and performing the search using one or more of the clustered sets of target genomes. In some embodiments, the clustering algorithm comprises a Markov cluster algorithm.

In some embodiments, the search is performed using BLAST, DIAMOND, HMMER, Exonerate, or ggsearch. In some embodiments, the search is restricted to one or more specific regions of the one or more target genomes. In some embodiments, the one or more specific regions comprise one or more biosynthetic gene clusters (BGCs). In some embodiments, the one or more BGCs in the one or more target genomes are predicted using a BGC search algorithm. In some embodiments, the BGC search algorithm comprises antiSMASH, SMURF, TOUCAN, or deepBGC.

In some embodiments, the one or more BGCs are predicted for the one or more target genomes by extracting sequence regions of a specified length that are proximal to gene sequences that match a known biosynthetic core synthase as determined using a sequence search tool. In some embodiments, the sequence search tool comprises BLAST, DIAMOND, HMMER, Exonerate, or ggsearch. In some embodiments, the one or more BGCs are predicted for the one or more query genomes using a Hidden Markov Model (HMM) of known core synthases. In some embodiments, the one or more BGCs are predicted for the one or more target genomes based on co-localization of protein sequence domains associated with known core synthases.

In some embodiments, the one or more homologous sequence-based metrics comprise percent sequence identity, percent sequence coverage, E-value, bitscore, HMM score, or any combination thereof. In some embodiments, the one or more predetermined homologous sequence-based metric thresholds comprise a percent sequence identity threshold, a percent sequence coverage threshold, an E-value threshold, a bitscore threshold, an HMM score threshold, or any combination thereof. In some embodiments, the one or more predetermined homologous sequence-based metric thresholds comprise a percent sequence identity threshold having a value of at least 20%, at least 30%, at least 40%, at least 50%, at least 60%, at least 70%, at least 75%, at least 80%, at least 85%, at least 90%, at least 95%, or at least 98%. In some embodiments, the one or more predetermined homologous sequence-based metric thresholds comprise a percent sequence coverage threshold having a value of at least 20%, at least 30%, at least 40%, at least 50%, at least 60%, at least 70%, at least 75%, at least 80%, at least 85%, at least 90%, at least 95%, or at least 98%. In some embodiments, the one or more predetermined homologous sequence-based metric thresholds comprise an E-value threshold having a value of less than 10, less than 9, less than 8, less than 7, less than 6, less than 5, less than 4, less than 3, less than 2, less than 1, less than 0.01, less than 0.001, less than 1e⁻¹⁰, less than 1e⁻²⁰, less than 1e⁻³⁰, less than 1e⁻⁴⁰, less than 1e⁻⁵⁰, less than 1e⁻⁶⁰, less than 1e⁻⁷⁰, less than 1e⁻⁸⁰, less than 1e⁻⁹⁰, or less than 1e⁻¹⁰⁰. In some embodiments, the one or more predetermined homologous sequence-based metric thresholds comprise a bitscore threshold having a value of at least 40, at least 50, at least 60, at least 70, at least 80, at least 90, at least 100, at least 250, at least 500, at least 1000, or at least 5000. In some embodiments, the one or more predetermined homologous sequence-based metric thresholds comprise an HMM score threshold having a value of at least 10, at least 25, at least 50, at least 100, at least 250, at least 500, at least 1000, or at least 5000.

In some embodiments, performing the search comprises: converting one or more query sequences that comprise protein sequences to nucleic acid sequences; performing a search of the one or more target genomes using the one or more query sequences that have been converted to nucleic acid sequences to identify homologous nucleic acid sequences based on a comparison of the one or more homologous sequence-based metrics for candidate pETaGs to the one or more predetermined homologous sequence-based metric thresholds; and comparing genomic coordinates of the homologous nucleic acid sequences with genomic coordinates corresponding to predicted protein sequences in the one or more target genomes. In some embodiments, if a homologous nucleic acid sequence overlaps a nucleic acid sequence corresponding to a single predicted protein sequence, and the overlap is greater than a specified nucleic acid sequence overlap threshold, the predicted protein sequence is reported as a pETaG. In some embodiments, if a homologous nucleic acid sequence overlaps nucleic acid sequences corresponding to a plurality of predicted proteins sequences, and the overlap for each is greater than a specified nucleic acid sequence overlap threshold, only one of the predicted protein sequences is reported as a pETaG.

In some embodiments, the predicted protein sequence reported as a pETaG is the predicted protein sequence for which the homologous nucleic acid sequence and the nucleic acid sequence corresponding to the predicted protein sequence exhibit the largest percent sequence identity, percent sequence coverage, E-value, or bitscore value. In some embodiments, the predicted protein sequence reported as a pETaG is the predicted protein sequence for which the homologous nucleic acid sequence and the nucleic acid sequence corresponding to the predicted protein sequence exhibit the longest overlapping sequence. In some embodiments, if a homologous nucleic acid sequence overlaps one or more nucleic acid sequences corresponding to one or more predicted proteins sequences, but the overlap for each is less than a specified nucleic acid sequence overlap threshold, the longest predicted protein sequence is reported as a pETaG. In some embodiments, if a homologous nucleic acid sequence does not overlap a nucleic acid sequence corresponding to a predicted protein sequence, the genomic coordinates of the homologous nucleic acid sequence are reported as a pETaG. In some embodiments, the specified nucleic acid sequence overlap threshold has a value of at least 20%, 30%, 40%, 50%, 60%, 70%, 75%, 80%, 85%, 90%, 95%, or 98%.

In some embodiments, the comparative genomics heatmap generated for a given pETaG comprises a plurality of cells arranged in a grid according to a first axis and a second axis, wherein the first axis corresponds to a plurality of different target genomes, wherein the plurality of different target genomes comprises a plurality of positive genomes each having an ortholog of an anchor gene sequence of a known BGC in one of the target genomes and a plurality of negative genomes that do not have an ortholog of the anchor gene sequence, wherein the second axis corresponds to a plurality of query gene sequences, or orthologs thereof, that are co-localized with the anchor gene sequence of the known BGC, wherein the putative embedded target gene (pETaG) is one of the plurality of co-localized query gene sequences, and wherein a numerical value for each cell is based on: (i) a presence or absence of an ortholog of the respective co-localized query gene sequence in the respective target genome; (ii) a sequence similarity of the ortholog to the respective co-localized query gene sequence; and (iii) whether the ortholog of the respective query gene sequence is co-localized with the ortholog of the anchor gene sequence in the respective genome.

In some embodiments, the computer-implemented method further comprises analyzing the comparative genomics heatmap, or underlying data thereof, using a trained machine learning model, wherein the machine-learning model is a classification model configured to output a probability for each of a plurality of predefined likelihood categories (e.g., categories of likelihood that the putative embedded gene is embedded in the gene cluster (e.g., a BGC)). In some embodiments, the classification model is a long short-term memory (LSTM) model. In some embodiments, the classification model is a convolutional neural network (CNN) model. In some embodiments, the classification model is a vision transformer model, a generative adversarial network model, a variational autoencoder model, or a latent diffusion model. In some embodiments, for example, the plurality of predefined likelihood categories comprise: (1) high likelihood; (2) more likely than not; (3) more unlikely than not; and (4) low likelihood.

Also disclosed herein are computer-implemented methods of determining a likelihood that a putative embedded target gene (pETaG) is a resistance gene against a secondary metabolite produced by a biosynthetic gene cluster (BGC) in a query genome, the methods comprising: a) determining one or more parameters selected from the following: i) a likelihood that the pETaG is associated with the BGC based on presence or absence of orthologs of each of a plurality of query genes that are co-localized with the BGC in a plurality of different genomes, wherein the plurality of genomes comprises a plurality of positive genomes comprising an ortholog of an anchor gene of the BGC, and a plurality of negative genomes that do not comprise an ortholog of the anchor gene of the BGC, wherein the anchor gene is known to associate with the BGC; ii) one or more phylogenetic features of the last common ancestor (LCA) of homologs of the pETaG in a phylogenetic tree of the plurality of genomes; iii) one or more scores indicative of co-occurrence of the ortholog of the pETaG and the ortholog of the anchor gene among the plurality of positive genomes; iv) one or more scores indicative of co-evolution of sequence divergence among orthologs of the pETaG with respect to sequence divergence among orthologs of the anchor gene in positive genomes comprising both an ortholog of the pETaG and an ortholog of the anchor gene; and v) one or more scores indicative of copy number of homologs of the pETaG in the plurality of positive genomes and copy number of homologs of the pETaG in the plurality of negative genomes; and b) determining the likelihood that the pETaG is a resistance gene against the secondary metabolite produced by the BGC based on the one or more parameters. In some embodiments, the computer-implemented further comprises predicting the probability that a pETaG is an actual ETaG using one or more of the parameters described above as input for a trained machine-learning model. In some embodiments, the trained machine-learning model is a trained neural network (e.g., an artificial neural network (ANN), a multilayer perceptron (MLP), a deep neural network (DNN), a convolutional neural network (CNN), and so forth). In some embodiments, the data can be utilized to train other types of machine-learning models (e.g. Bayesian inference, decision tree-based methods such as XGBoost or Random Forest, and so forth). In some embodiments, the data can be utilized to train logistic regression models or other types of supervised models.

In some embodiments, the pETaG is co-localized with the BGC in the query genome. In some embodiments, the pETaG is not involved in production of the secondary metabolite by the BGC. In some embodiments, the anchor gene is the core synthase gene of the BGC.

In some embodiments, the computer-implemented method comprises determining a likelihood for each of a plurality of pETaGs that the pETaG is a resistance gene against a secondary metabolite produced by a BGC in a target genome. In some embodiments, the computer-implemented comprises: a) identifying putative BGCs in a plurality of genomes having pairwise sequence similarity above a threshold value; b) identifying non-biosynthetic genes that are co-localized with orthologs of the anchor gene in the putative BGCs, wherein the non-biosynthetic genes are homologous to any one of a plurality of query genes in an organism of interest, and wherein the non-biosynthetic genes are not involved in production of secondary metabolites by the BGCs; c) for each of the plurality of query genes, identifying the non-biosynthetic gene encoding a protein having the highest sequence similarity to the protein of the respective target gene as the pETaG and the genome encoding the non-biosynthetic gene as the target genome; and d) for each of the plurality of query genes, determining a likelihood that the respective pETaG is a resistance gene against a secondary metabolite produced by the respective BGC in the respective target genome. In some embodiments, the computer-implemented method comprises: a) clustering genomes in a database into a plurality of clusters each comprising genomes having pairwise sequence similarity above a threshold value; b) for each of the plurality of clusters: i) identifying non-biosynthetic genes that are co-localized with orthologs of the anchor gene in the putative BGCs, wherein the non-biosynthetic genes are homologous to any one of a plurality of query genes in an organism of interest, and wherein the non-biosynthetic genes are not involved in production of secondary metabolites by the BGCs; ii) for each of the plurality of query genes, identifying the non-biosynthetic gene encoding a protein having the highest sequence similarity to the protein of the respective query gene as a candidate pETaG; and c) clustering the candidate pETaGs into a plurality of clusters based on sequence similarities among the pETaGs, and identifying the candidate pETaG encoding a protein having the highest sequence similarity to the protein of the respective query gene in each cluster as the pETaG and the respective genome encoding the pETaG as the target genome; and d) for each of the plurality of query genes, determining a likelihood that the respective pETaG is a resistance gene against a secondary metabolite produced by the respective BGC in the respective target genome.

In some embodiments, the threshold value is at least 70%, at least 75%, at least 80%, at least 85%, at least 90%, at least 95%, or at least at least 98% pairwise sequence similarity. In some embodiments, each of the identified non-biosynthetic genes encodes a protein having at least about 30% sequence identity to a protein encoded by the respective query gene.

In some embodiments, the plurality of query genes are all protein-coding genes in the organism of interest. In some embodiments, the organism of interest is a mammal. In some embodiments, the organism of interest is human. In some embodiments, the organism of interest is a reptile, a bird, an amphibian, an animal, a plant, a fungus, or a bacterium.

In some embodiments, the plurality of genomes are fungal genomes. In some embodiments, the plurality of genomes are bacterial genomes. In some embodiments, the plurality of genomes are plant genomes.

In some embodiments, each of the plurality of clusters comprise about 10 to about 100 genomes.

Disclosed herein are computer-implemented methods of identifying a druggable target in an organism of interest, comprising performing any one of the methods described herein, and identifying a query gene as a druggable target based on the likelihood that the respective pETaG of the query gene is a resistance gene against a secondary metabolite produced by the BGC in the target genome. In some embodiments, the computer-implemented method further comprises identifying the secondary metabolite or an analog thereof as a small molecule modulator of the query gene or a protein encoded by the query gene. In some embodiments, the computer-implemented method of claim 62, further comprising contacting the secondary metabolite or analog thereof with a protein encoded by the query gene, and detecting an activity of the protein encoded by the query gene. In some embodiments, the number of the positive genomes is equal to the number of the negative genomes. In some embodiments, the computer-implemented method comprises selecting a plurality of positive genomes and a plurality of negative genomes from a database of genomes. In some embodiments, the computer-implemented method comprises clustering the database of genomes into a plurality of clusters based on sequence similarities, and selecting one positive genome per cluster to provide the plurality of positive genomes. In some embodiments, the computer-implemented method comprises selecting a negative genome that has highest sequence similarity to each positive genome in the cluster. In some embodiments, the average pairwise percentage sequence identities of orthologs of one or more single copy genes in the positive genomes is no more than about 95%, and/or the average pairwise percentage sequence identities of orthologs of one or more single copy genes in the negative genomes is no more than about 95%. In some embodiments, the number of the positive genomes is at least 5.

In some embodiments, the one or more parameters comprises a likelihood that the pETaG is associated with the BGC based on presence or absence of orthologs of each of a plurality of query genes in the BGC in a plurality of different genomes. In some embodiments, determining a likelihood that the pETaG is associated with the BGC comprises: a) receiving a grid representation comprising a plurality of cells arranged according to a first axis and a second axis, wherein the first axis corresponds to the plurality of genomes, wherein the second axis corresponds to the plurality of query genes in the BGC in the query genome, and wherein each cell is based on: i) the presence or absence of an ortholog of the respective query gene in the respective genome; ii) sequence similarity of the ortholog to the respective query gene; and iii) whether the ortholog of the respective query gene is co-localized with the ortholog of the anchor gene in the respective genome; and b) inputting the grid representation into a machine-learning model, wherein the machine-learning model is trained to determine a likelihood that the pETaG is associated with the BGC based on values of the plurality of cells in the grid representation, thereby providing the likelihood that the pETaG is associated with the BGC.

In some embodiments, the computer-implemented method further comprises: a) identifying a putative BGC comprising a pETaG from a library of putative BGCs from a plurality of genomes and identifying the longest biosynthetic gene in the putative BGC as the core synthase gene; b) obtaining a plurality of positive genomes comprising an ortholog of the core synthase gene and a plurality of negative genomes that do not comprise an ortholog of the core synthase gene, wherein the plurality of positive genomes have pairwise sequence similarities no more than a threshold value, and wherein the plurality of negative genomes are selected based on sequence similarity to the plurality of positive genomes; and c) creating a grid representation comprising a plurality of cells arranged according to a first axis and a second axis, wherein the first axis corresponds to all protein-coding genes that are co-localized with the core synthase gene in the putative BGC in the query genome, and the second axis corresponds to the plurality of positive genomes and the plurality of negative genomes, wherein each cell is computed based on: i) the presence or absence of an ortholog of the respective protein-coding gene in the respective genome; ii) sequence similarity of the ortholog to the respective protein-coding gene; and iii) whether the ortholog of the respective protein-coding gene is co-localized with the ortholog of the core synthase gene in the respective genome.

In some embodiments, the machine-learning model is a classification model configured to output a probability for each of a plurality of predefined likelihood categories. In some embodiments, the classification model is a long short-term memory (LSTM) model. In some embodiments, the classification model is a convolutional neural network (CNN). In some embodiments, the classification model is a vision transformer model, a generative adversarial network model, a variational autoencoder model, or a latent diffusion model. In some embodiments, the plurality of predefined likelihood categories comprise: (1) high likelihood; (2) more likely than not; (3) more unlikely than not; and (4) low likelihood.

In some embodiments, the one or more parameters comprises one or more phylogenetic features of the last common ancestor (LCA) of homologs of the pETaG in a phylogenetic tree of the plurality of positive and negative genomes. In some embodiments, the one or more phylogenetic features are selected from the group consisting of average copy number difference (CND) between genes in the plurality of positive genomes and genes in the plurality of negative genomes and values determined from a plurality of positive genomes, ratio mean to LCA, ratio standard deviation to LCA, mean ratio of neighbor distance, standard deviation of ratio of neighbor distance, and sum of ratio clade. In some embodiments, the one or more parameters comprise one or more scores indicative of co-occurrence of the ortholog of the pETaG and the ortholog of the anchor gene among the plurality of positive genomes. In some embodiments, the one or more scores indicative of co-occurrence are selected from the group consisting of co-occurrence pETaG distance, co-occurrence pETaG rank, co-occurrence core distance, and co-occurrence core rank. In some embodiments, the one or more parameters comprise one or more scores indicative of co-evolution of sequence divergence among orthologs of the pETaG with respect to sequence divergence among orthologs of the anchor gene in positive genomes comprising both an ortholog of the pETaG and an ortholog of the anchor gene. In some embodiments, the one or more scores indicative of co-evolution are selected from the group consisting of co-evolution correlation, co-evolution rank, and co-evolution slope. In some embodiments, the one or more parameters further comprise one or more features of the plurality of positive genomes and the plurality of negative genomes. In some embodiments, the one or more features are selected from the group consisting of the number of the positive genomes, mean pairwise genomic identity (PGI) among the positive genomes, standard deviation of PGI among the positive genomes, the number of the negative genomes, mean PGI among the negative genomes, and standard deviation of PGI among the negative genomes.

In some embodiments, said determining the likelihood based on the one or more parameters comprises inputting the one or more features into a machine-learning model, wherein the machine-learning model has been trained to determine a likelihood that the pETaG is a resistance gene. In some embodiments, the machine-learning model is a deep learning model. In some embodiments, the machine-learning model is a decision tree model. In some embodiments, the machine-learning model is a Bayesian inference model. In some embodiments, one or more features can be utilized to train logistic regression models or other types of supervised models. In some embodiments, whether a gene is co-localized with an anchor gene of a BGC is determined using antiSMASH. In some embodiments, whether a gene is co-localized with an anchor gene of a BGC is determined based on whether the gene is located within a proximity distance from the anchor gene. In some embodiments, the proximity zone is no more than about 50 kb. In some embodiments, the proximity zone is about 20 kb.

Disclosed herein are systems comprising: one or more processors; and a memory communicatively coupled to the one or more processors and configured to store instructions that, when executed by the one or more processors, cause the system to: i) receive as input one or more query sequences, or proxies thereof; ii) receive as input a selection of one or more target genomes; iii) perform a search of the one or more target genomes using the one or more query sequences, or proxies thereof, to identify putative embedded target gene (pETaG) sequences that are homologs of the one or more query sequences based on a comparison of one or more homologous sequence-based metrics for candidate pETaGs to one or more predetermined homologous sequence-based metric thresholds; and iv) determine whether or not a given pETaG is an actual ETaG based on a comparative genomics analysis of a plurality of genomes related to the one or more target genomes.

Also disclosed herein are systems comprising: one or more processors; and a memory communicatively coupled to the one or more processors and configured to store instructions that, when executed by the one or more processors, cause the system to perform a method of determining a likelihood that a putative embedded target gene (pETaG) is a resistance gene against a secondary metabolite produced by a biosynthetic gene cluster (BGC) in a query genome, the method comprising: a) determining one or more parameters selected from the following: i) a likelihood that the pETaG is associated with the BGC based on presence or absence of orthologs of each of a plurality of query genes that are co-localized with the BGC in a plurality of different genomes, wherein the plurality of genomes comprises a plurality of positive genomes comprising an ortholog of an anchor gene of the BGC, and a plurality of negative genomes that do not comprise an ortholog of the anchor gene of the BGC, wherein the anchor gene is known to associate with the BGC; ii) one or more phylogenetic features of the last common ancestor (LCA) of homologs of the pETaG in a phylogenetic tree of the plurality of genomes; iii) one or more scores indicative of co-occurrence of the ortholog of the pETaG and the ortholog of the anchor gene among the plurality of positive genomes; iv) one or more scores indicative of co-evolution of sequence divergence among orthologs of the pETaG with respect to sequence divergence among orthologs of the anchor gene in positive genomes comprising both an ortholog of the pETaG and an ortholog of the anchor gene; and v) one or more scores indicative of copy number of homologs of the pETaG in the plurality of positive genomes and copy number of homologs of the pETaG in the plurality of negative genomes; and b) determining the likelihood that the pETaG is a resistance gene against the secondary metabolite produced by the BGC based on the one or more parameters. In some embodiments, the method performed by the system further comprises predicting the probability that a pETaG is an actual ETaG using one or more of the parameters described above as input for a trained machine-learning model. In some embodiments, the trained machine-learning model is a trained neural network (e.g., an artificial neural network (ANN), a multilayer perceptron (MLP), a deep neural network (DNN), a convolutional neural network (CNN), and so forth). In some embodiments, the data can be utilized to train other types of machine-learning models (e.g. Bayesian inference, decision tree-based methods such as XGBoost or Random Forest, and so forth). In some embodiments, the data can be utilized to train logistic regression models or other types of supervised models.

Disclosed herein are systems comprising: one or more processors; and a memory communicatively coupled to the one or more processors and configured to store instructions that, when executed by the one or more processors, cause the system to perform any of the methods described herein.

Disclosed herein are non-transitory computer-readable storage media storing one or more programs, the one or more programs comprising instructions, which when executed by one or more processors of an electronic device, cause the electronic device to perform any of the methods described herein.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE FIGURES

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 illustrates an exemplary putative biosynthetic gene cluster (BGC) as predicted by antiSMASH.

FIG. 2 provides a non-limiting example of a process flowchart for identifying and evaluating putative embedded target genes (pETaGs).

FIG. 3 provides an exemplary illustration of positive genomes (i.e., genomes that contain the core synthase gene sequence) and negative genomes (i.e., genomes that do not contain the core synthase gene sequence).

FIG. 4 provides a non-limiting example of a process flowchart for identifying and evaluating putative embedded target genes (pETaGs).

FIG. 5 provides an exemplary illustration of different outcomes for evaluating targeted pETaG search results.

FIG. 6 illustrates an exemplary method for generating a grid representation (e.g., heat map) of comparative genomics data.

FIG. 7 illustrates an exemplary method for determining a likelihood that a putative embedded gene co-localized with a core synthase gene of a BGC in a query genome is associated with the BGC, according to some examples.

FIGS. 8A-1 to 8A-3 illustrate an exemplary comparative genomics heat map.

FIGS. 8B-1 and 8B-2 illustrate an exemplary long short-term memory (LSTM) model used for classification of an input comparative genomics heat map into one of four likelihood categories (i.e., tiers).

FIGS. 8C-1 and 8C-2 illustrate an example of an output of the LSTM model illustrated in FIGS. 8B-1 and 8B-2.

FIG. 9A illustrates a table comparing manual classification versus machine-learning based classification of comparative genomics heat maps for “Tier A+”, “Tier 1”, “Tier 2”, and “Tier 3” categories, respectively.

FIG. 9B illustrates a table comparing manual classification versus machine-learning based classification of comparative genomics heat maps for “Tier A+”, “Tier 1”, “Tier 2”, and “Tier 3”, including a positive predictive value, a negative predictive value, a sensitivity value, and a specificity value.

FIGS. 10A and 10B illustrate an exemplary comparative genomics heat map of a BGC for lovastatin that identifies the true borders of the lovastatin BGC as compared to the lovastatin BGC predicted by antiSMASH.

FIGS. 11A-1 and 11A-2 illustrate an exemplary comparative genomics heat map manually reviewed and classified as “Tier A+”.

FIGS. 11B-1 and 11B-2 illustrate an exemplary comparative genomics heat map manually reviewed and classified as “Tier 1”.

FIGS. 11C-1 and 11C-2 illustrate an exemplary comparative genomics heat map manually reviewed and classified as “Tier 2”.

FIGS. 11D-1 and 11D-2 illustrate an exemplary comparative genomics heat map manually reviewed and classified as “Tier 3”.

FIG. 12A illustrates a data table of a set of features (e.g., up to 27 features or more) that may be organized into a data table and utilized to train a machine-learning model.

FIG. 12B illustrates an initial training phase of a neural network trained to output a probability value that a putative embedded gene (e.g., a pETaG) is associated with a BGC (i.e., an “embedded gene probability value” (e.g., an “ETaG probability value”)).

FIG. 12C illustrates a further training phase of a neural network trained to output an embedded gene probability value (e.g., an “ETaG probability value”).

FIG. 12D illustrates an inference phase of a neural network trained to output an ETaG probability value.

FIG. 12E illustrates an example data table including the unknown input data set of features and the corresponding outputs of ETaG probability values for each of the features.

FIG. 13 provides an exemplary illustration of the workflow for calculating phylogenetic features using a custom software algorithm (PhyloCCC).

FIG. 14 provides a non-limiting example of phylogenetic features calculated from a Lovastatin ETaG and a selected set of positive and negative genomes.

FIG. 15 provides a non-limiting example of a workflow for evaluating co-evolution when percent identities are used to compare the co-evolution between pairs of COGs.

FIG. 16 provides a non-limiting example of the number of units used per hidden layer for a deep learning model trained to evaluate the likelihood that a putative ETaG is an ETaG.

FIG. 17 provides a non-limiting example of performance data (test loss) for a deep learning model trained to evaluate the likelihood that a putative ETaG is an ETaG.

FIG. 18 provides a non-limiting example of performance data (test specificity) for a deep learning model trained to evaluate the likelihood that a putative ETaG is an ETaG.

FIG. 19 provides a non-limiting example of performance data (test sensitivity) for a deep learning model trained to evaluate the likelihood that a putative ETaG is an ETaG.

FIG. 20 provides a non-limiting example of performance data (test precision) for a deep learning model trained to evaluate the likelihood that a putative ETaG is an ETaG.

### DETAILED DESCRIPTION

The present disclosure provides comparative genomics methods and systems for identifying orthologs of genes associated with a biosynthetic gene cluster (BGC) in a first genome that occur in one or more target genomes. In particular, the disclosed methods and systems may be used to identify putative embedded target genes (pETaGs) that are associated with, e.g., a resistance mechanism, in the one or more target genomes based on a specified set of sequence homology search criteria. The pETaGs thus identified may then be filtered and evaluated for the likelihood that they are actual ETaGs associated with, e.g., the resistance mechanism, in the host organism(s) from which the one or more target genomes are derived.

In some instances, pETaGs may be filtered and evaluated based on a comparative genomics analysis for each pETaG using a grid representation (e.g., a heat map) of genomic data associated with a given pETaG. The grid representation enables visual and/or machine-learning based evaluation of co-occurrence and co-localization between genes found in proximity to biosynthetic genes, biosynthetic gene clusters (BGCs), or other gene(s) of interest, as will be described in more detail below.

In some instances, pETaGs may be filtered and evaluated based on a variety of comparative genomics analysis-based evolutionary metrics. Examples of such evolutionary metrics include, but are not limited to, phylogenetic features, co-occurrence features, co-evolution features, and genome data set features, which may be determined for a plurality of genomes comprising both positive genomes (i.e., genomes that contain a core synthase gene sequence) and negative genomes (i.e., genomes that do not contain the core synthase gene sequence), as will be described in more detail below.

In some instances, data derived from comparative genomics-based heatmaps and/or evolutionary metrics for pETAGs may be further processed using, e.g., empirical algorithms and/or machine learning-based models, to determine a likelihood score or probability that a given pETaG is an actual ETaG, as will be described in more detail below. The output from such an analysis may then be used to compile a Target Look-up Table (e.g., an array of data that collects the sequence features and values or ranges of evolutionary metrics for each pETaG, and its probability to be an actual ETaG).

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly indicates otherwise. Any reference to “or” herein is intended to encompass “and/or” unless otherwise stated, and encompasses any and all possible combinations of one or more of the associated listed items.

As used herein, the terms “includes, “including,” “comprises,” and/or “comprising” specify the presence of stated features, integers, steps, operations, elements, components, and/or units but do not preclude the presence or addition of one or more other features, integers, steps, operations, elements, components, units, and/or groups thereof.

As used herein, the term “about” a number refers to that number plus or minus 10% of that number. The term ‘about’ when used in the context of a range refers to that range minus 10% of its lowest value and plus 10% of its greatest value.

As used herein, a “secondary metabolite” refers to an organic small molecule compound produced by archaea, bacteria, fungi or plants, which are not directly involved in the normal growth, development, or reproduction of the host organism, but are required for interaction of the host organism with its environment. Secondary metabolites are also known as natural products or genetically encoded small molecules. The term “secondary metabolite” is used interchangeably herein with “biosynthetic product” when referring to the product of a biosynthetic gene cluster.

The terms “biosynthetic gene cluster” or “BGC” are used herein interchangeably to refer to a locally clustered group of one or more genes that together encode a biosynthetic pathway for the production of a secondary metabolite. BGCs contain genes encoding signature biosynthetic proteins that are characteristic of each type of BGCs. The longest biosynthetic gene in a BGC is referred to herein as the “core synthase gene” of a BGC. In addition to genes involved in the biosynthesis of a secondary metabolite, a BGC may also include non-biosynthetic genes, i.e., genes that encode products that are not involved in the biosynthesis of a secondary metabolite, which are interspersed among the biosynthetic genes. The non-biosynthetic genes are referred herein as being “associated” or “embedded” with the BGC if their products are functionally related to the secondary metabolite of the BGC. The term “anchor gene” as used herein refers to a biosynthetic or non-biosynthetic gene that is co-localized with a BGC and is known to be functionally related (i.e., associated) with the BGC.

The term “co-localize” refers to the presence of two or more genes in close spatial proximity, such as no more than about 200 kb apart, no more than about 100 kb apart, no more than about 50 kb apart, no more than about 40 kb apart, no more than about 30 kb apart, no more than about 20 kb apart, no more than about 10 kb apart, no more than about 5 kb apart, or less, in a genome.

The term “homolog” refers to a gene that is part of a group of genes that are related by descent from a common ancestor (i.e., the gene sequences (i.e., nucleic acid sequences) of the group of genes and/or the sequences of their protein products are inherited from a common origin). Homologs may arise through speciation events (giving rise to “orthologs”), or through gene duplication events, or through horizontal gene transfer events. Homologs may be identified by phylogenetic methods, through identification of common functional domains in the aligned nucleic acid or protein sequences, or through sequence comparisons.

The term “ortholog” refers a gene that is part of a group of genes that are predicted to have evolved from a common ancestral gene by speciation.

The terms “bidirectional best hit” and “BBH” are used herein interchangeably to refer to the relationship between a pair of genes in two genomes (i.e., a first gene in a first genome and a second gene is a second genome) wherein the first gene or its protein product has been identified as having the most similar sequence in the first genome as compared to the second gene or its protein product in the second genome, and wherein the second gene or its protein product has been identified as having the most similar sequence in the second genome as compared to the first gene or its protein product in the first genome. The first gene is the bidirectional best hit (BBH) of the second gene, and the second gene is the bidirectional best hit (BBH) of the first gene. BBH is a commonly used method to infer orthology.

As used herein, “sequence similarity” between two genes means similarity of either the nucleic acid (e.g., mRNA) sequences encoded by the genes or the amino acid sequences of the gene products.

“Percent (%) sequence identity” or “percent (%) sequence homology” with respect to the nucleic acid sequences (or protein sequences) described herein is defined as the percentage of nucleotide residues (or amino acid residues) in a candidate sequence that are identical or homologous with the nucleotide residues (or amino acid residues) in the oligonucleotide (or polypeptide) with which the candidate sequence is being compared, after aligning the sequences and considering any conservative substitutions as part of the sequence identity. Homology between different amino acid residues in a polypeptide is determined based on a substitution matrix, such as the BLOSUM (BLOcks Substitution MAtrixAlignment). Methods for aligning sequences and determining percent sequence identity or percent sequence homology for nucleic acid or protein sequences are well known to those of skill in the art. Examples of publicly available computer software that may be used include, but are not limited to, BLAST (Basic Local Alignment Search Tool; software for comparing the amino-acid sequences of proteins or the nucleotide sequences of DNA and/or RNA molecules), BLAST-2, ALIGN or Megalign (DNASTAR) software. Any of a variety of suitable parameters for measuring sequence alignment and determining percent sequence identity or homology may be determined by those of skill in the art, including use of algorithms required to achieve maximal alignment over the full length of the sequences being compared.

Certain aspects of the present disclosure include process steps and instructions described herein in the form of an algorithm. It should be noted that the process steps and instructions of the present disclosure may be embodied in software, firmware, and/or hardware, and when embodied in software, may be downloaded to reside on and be operated from different platforms used by a variety of operating systems. Unless specifically stated otherwise in the following disclosure, it will be appreciated that descriptions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

### Methods for Discovery of Embedded Target Genes in Biosynthetic Gene Clusters

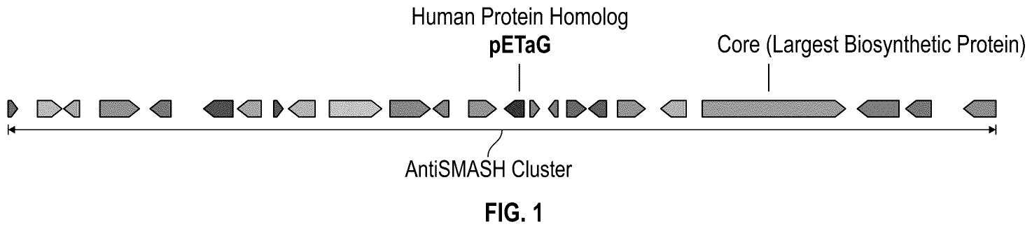

The systems and methods described herein relate to identification of genes associated with a biosynthetic gene cluster (BGC). FIG. 1 illustrates an exemplary putative biosynthetic gene cluster (BGC) as predicted by antiSMASH—a genomics database search tool that enables rapid genome-wide identification, annotation, and analysis of secondary metabolite biosynthesis gene clusters in, e.g., bacterial, plant, and fungal genomes.

As indicated in FIG. 1, the putative BGC may include a series of genes that encode for biosynthetic enzymes (the longest of which is referred to herein as a “core biosynthetic protein” or “core synthase”) in a biosynthetic pathway for the production of a secondary metabolite, as well as a series of interspersed, non-biosynthetic genes. Exemplary BGCs include, but are not limited to, biosynthetic gene clusters for the synthesis of non-ribosomal peptide synthetases (NRPS), polyketide synthases (PKS), terpenes, and bacteriocins. See, for example, Keller N, “Fungal secondary metabolism: regulation, function and drug discovery.” Nature Reviews Microbiology 17.3 (2019): 167-180 and Fischbach M. and Voigt C. A., PROKARYOTIC GENE CLUSTERS: A RICH TOOLBOX FOR SYNTHETIC BIOLOGY. In: Institute of Medicine (US) Forum on Microbial Threats. The Science and Applications of Synthetic and Systems Biology: Workshop Summary. Washington (DC): National Academies Press (US); 2011. A21.

In some cases, non-biosynthetic genes that co-localize with the biosynthetic genes in a BGC may be homologs of human proteins, including therapeutic targets of interests. In some instances, the non-biosynthetic genes that co-localize with the BGC may comprise a homolog of a mammalian gene, a reptilian gene, an avian gene, an amphibian gene, etc., or a gene of interest from any other organism, including genes encoding for veterinarian therapeutic targets of interest. In some embodiments, the non-biosynthetic genes that co-localize with the BGC may be a homolog of a fungal protein, including fungicidal targets of interest. In some embodiments, the non-biosynthetic genes that co-localize with the BGC may be a homolog of a plant protein, including herbicidal targets of interest. In some embodiments, the non-biosynthetic genes that co-localize with the BGC may be a homolog of a bacterial protein, including microbial targets of interest. In some instances, non-biosynthetic genes may be functionally related to the secondary metabolite produced by the BGC. In some instances, non-biosynthetic genes that are functionally related to the secondary metabolite produced by the BGC may be functionally related to a resistance mechanism that protects the host organism from the effects of the secondary metabolite. In some instances, non-biosynthetic genes may encode functionally irrelevant protein products. A non-biosynthetic gene in a BGC which is a homolog of a human protein of interest, is a putative embedded target gene (pETaG). The methods described herein enable the identification of pETaGs in one or more target genomes based on a known (or query) ETaG sequence in a first (or reference) genome (e.g., a human genome, a mammalian genome, a reptilian genome, an avian genome, an amphibian genome, a plant genome, a bacterial genome, a fungal genome, or any other genome of interest), and leverage comparative genomics to determine the likelihood that a given pETaG is in fact a true ETaG associated with a BGC.

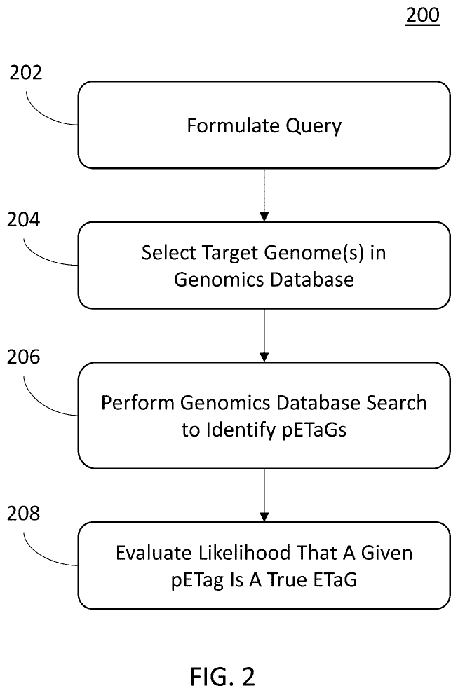

FIG. 2 provides a non-limiting example of a process 200 for formulating a query and conducting a search of one or more query genomes (or target genomes) in one or more genomic databases to identify pETaGs, which may then be evaluated for the likelihood that a given pETaG is a true pETaG. Process 200 can be performed, for example, as a computer-implemented method using software running on one or more processors of one or more electronic devices, computers, or computing platforms. In some examples, process 100 is performed using a client-server system, and the blocks of process 100 are divided up in any manner between the server and a client device. In other examples, the blocks of process 100 are divided up between the server and multiple client devices. Thus, while portions of process 100 are described herein as being performed by particular devices of a client-server system, it will be appreciated that process 100 is not so limited. In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

At step 202 in FIG. 2, a search query comprising one or more known query sequences (e.g., one or more gene sequences of interest) is formulated. A search query may comprise one or more query sequences (or target sequences, e.g., one or more known ETaG sequences), or proxies thereof. For example, a search query may comprise one or more protein (amino acid) sequences, one or more nucleic acid (nucleotide) sequences, one or more Universal Protein Resource (Uniprot) identification numbers, one or more profile Hidden Markov Models (pHMMs; i.e., probabilistic models that capture the biological diversity of the protein domains based on a position-dependent scoring of multiple sequence alignments for the corresponding protein or nucleic acid sequences), a specified set of protein or nucleic acid sequence domains, or any combination thereof. A search query may comprise one or more query sequences (e.g., one or more gene sequences of interest), or proxies thereof, selected from, for example, an archaea genome, a bacterial genome, a fungal genome, a plant genome, an animal genome, a human genome, or any combination thereof. A search query may comprise one or more query sequences (e.g., one or more gene sequences of interest) selected, for example, according to participation of the corresponding protein products in a cell biosynthetic pathway, a cell signaling pathway, a disease state (e.g., a cancer, an immunological disease, and infectious disease, or a rare disease), or any combination thereof.

In some instances, e.g., in a target agnostic search, all protein sequences (as an example) from one or more genomes (e.g., from one or more organisms) may be selected as query sequences for the query. In some instances, e.g., in a targeted search, one or more specific protein sequences (as an example) from one or more genomes may be selected as query sequences for the query. In some instances, a search query may comprise 1, 2, 3, 4, 5, 6, 7, 8, 9, 10, 100, 1000, 10,000, 20,000, 30,000, 40,000, 50,000, 60,000, 70,000, 80,000, 90,000, 100,000, or more than 100,000 query sequences (or any number of query sequences within this range). The formulation of search queries will be discussed in more detail below.

At step 204 in FIG. 2, the one or more target genomes to be searched for homologous sequences are selected. In some instances, target genomes can be selected from organisms in any kingdom of life for which, e.g., secondary metabolite molecules, are known to be produced (e.g., archaea, bacteria, fungi, plants, etc.). In some instances, target genomes may be selected from organisms in any kingdom of life, for example, archaea, bacteria, fungi, plants, animals, humans, or any combination thereof. In some instances, an individual genome can be used as a target genome. In some instances, a plurality of genomes drawn from the same or from different kingdoms of life can be used as target genomes. In some instances, 1, 2, 3, 4, 5, 6, 7, 8, 9, 10, 15, 20, 25, 30, 35, 40, 45, 50, 60, 70, 80, 90, 100, 150, 200, 250, 500, 1,000, 5,000, 10,000, 20,000, 30,000, 40,000, 50,000, 60,000, 70,000, 80,000, 90,000, 100,000, or more than 100,000 genomes (or any number of genomes within this range) may be selected for a search. In some instances, genomes may be grouped according to, e.g., pairwise percent sequence identity or phylogenetic distance, prior to performing a search. The selection and grouping of target genomes to be searched will be discussed in more detail below.

At step 206 in FIG. 2, a search is performed using one or more genomic and/or proteomic databases to identify sequences that are homologous to the one or more query sequences (e.g., putative embedded target genes (pETaGs)) in the one or more target genomes.

The identification of homologous sequences may comprise alignment of query sequences to sequences in the one or more target genomes, and determination of one or more homologous sequence-based metrics. In some instances, 1, 2, 3, 4, 5, 6, 7, 8, 9, 10, 100, 500, 1000, 5,000, 10,000, 20,000, 30,000, 40,000, 50,000, 60,000, 70,000, 80,000, 90,000, 100,000, or more than 100,000 genomes (or any number of genomes within this range) may be selected as target genomes. Examples of suitable homologous sequence-based metrics include, but are not limited to, percent sequence identity, percent sequence coverage, E-value (a parameter that indicates the number of expected hits of equivalent sequence similarity score that could be found by chance), bitscore (a measure of sequence similarity that is independent of query sequence length and database size, and is normalized based on raw pairwise alignment scores), HMM score (a measure from Hidden Markov Models that capture biological diversity of the protein domains based on a position-dependent scoring of multiple sequence alignments for the corresponding protein or nucleic acid sequences), or any combination thereof. Suitable search methods will be described in more detail below.

At step 208 in FIG. 2, pETaGs identified by the search are evaluated to determine the likelihood that a given pETaG is a true ETaG. Evaluation of pETaGs may be based on, e.g., the use of comparative genomics heatmaps and/or calculation of genome-derived metrics to determine whether a candidate pETaG is associated with a candidate core synthase (or with a candidate BGC). In some instances, the comparative genomics analysis used to evaluate pETaGs may be based on identifying a set of positive genomes and negative genomes. Methods for evaluating pETaGs to identify true ETaGs will be described in more detail below.

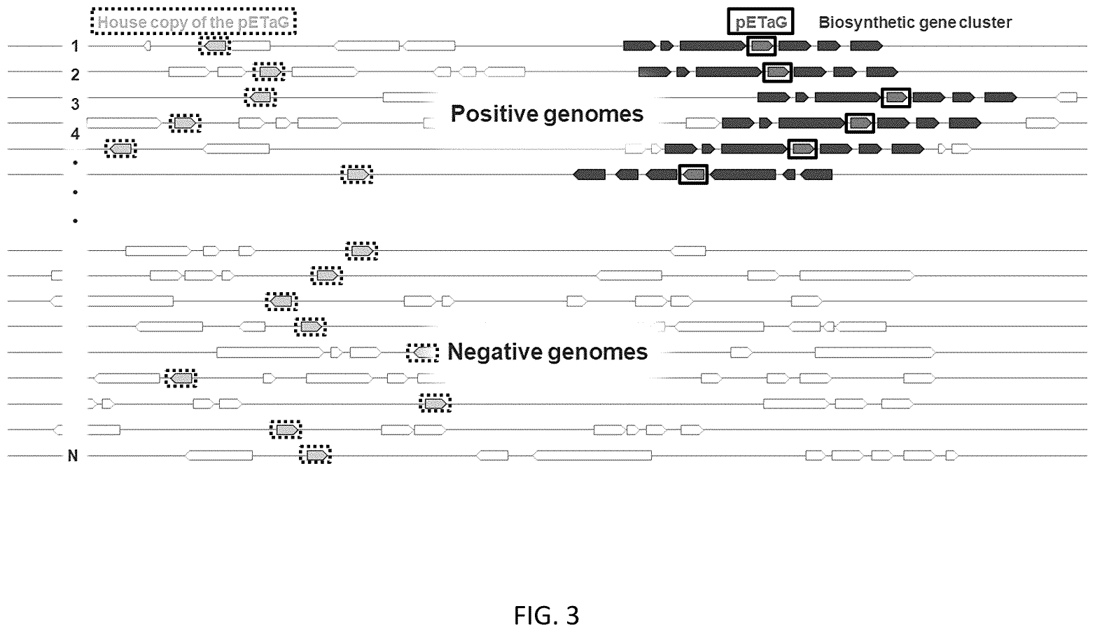

FIG. 3 provides a schematic illustration of positive genomes (i.e., genomes that contain a core synthase gene sequence) and negative genomes (i.e., genomes that do not contain the core synthase gene sequence). In this example, genomes 1, 2, 3, 4, . . . , N have been aligned and searched to identify a pETaG as well as other genes in a putative BGC. Genomes that include an embedded copy of the pETaG sequence as well as other genes in the BGC are considered to be “positive” genomes. In addition to the copy of the pETaG sequence that is embedded within the BGC region, some positive genomes may include an additional copy of the pETaG sequence (e.g., a house-keeping copy). In some cases, the presence of one or more additional copies of the pETaG sequence (e.g., house-keeping copies) may be indicative of a pETaG that is involved in a resistance mechanism. Genomes that lack the BGC are considered to be “negative” genomes. As illustrated, some negative genomes may include a non-embedded pETaG sequence (e.g., a house-keeping copy only).

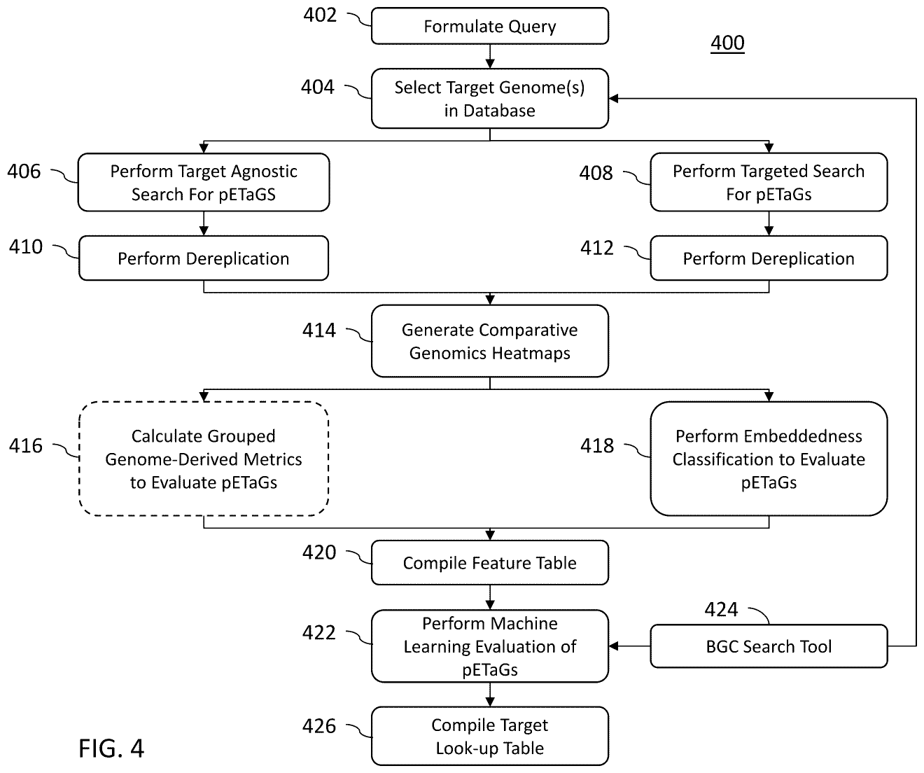

FIG. 4 provides another non-limiting example of a flowchart for a process 400 for identifying and evaluating putative embedded target genes (pETaGs).

Formulation of search queries: At step 402 in FIG. 4, a search query for identifying putative ETaGs is formulated. In some instances, a search may comprise a target agnostic search in which, for example, all of the proteins in one or more organisms are selected as query sequences. In some instances, a search may comprise a targeted search in which, for example, one or more specific proteins from one or more organisms are selected as query sequences. In some instances, a search query may comprise 1, 2, 3, 4, 5, 6, 7, 8, 9, 10, 100, 1000, or more than 1000 known query sequences. In some instances, query targets (for either target agnostic or targeted searches) may be selected from 1, 2, 3, 4, 5, 6, 7, 8, 9, 10, or more than 10 organisms. The one of more organisms selected as sources of query targets can be drawn from any kingdom of life, e.g., animals, plants, fungi, bacteria, archaea, etc.

Query targets, for either target agnostic or targeted searches, may be specified as a protein sequence (amino acid), a gene sequence (nucleotide), a Uniprot ID, a profile Hidden Markov Model (pHMM), a set of specified protein or nucleic acid sequence domains, or any combination thereof. In some instances, queries may be grouped by participation of the proteins of interest in specific pathways, by the sequence similarity between the proteins of interest, by their involvement in certain diseases, etc.

Selection of target genomes: At step 404 in FIG. 4, the target genomes to be searched for homologs of the proteins of interest are selected from a genomic database. Target genomes can be selected for organisms drawn from any kingdom of life (e.g., animals, plants, fungi, bacteria, archaea, etc.) in which secondary metabolite molecules are known to be produced. In some instances, an individual genome may be used as a target genome. In some instances, all genomes in a given database may be used as target genomes. In some instances, 1, 2, 3, 4, 5, 6, 7, 8, 9, 10, 100, 1000, 5,000, 10,000, 20,000, 30,000, 40,000, 50,000, 60,000, 70,000, 80,000, 90,000, 100,000, or more than 100,000 genomes (or any number of genomes within this range) may be selected as target genomes. Examples of suitable genomic databases include, but are not limited to, UniProt Knowledgebase (a protein sequence and function database), Swiss-Prot (a curated protein sequence database), GenBank (an annotated collection of all publicly available DNA sequences), MycoCosm (Joint Genome Institute), PhycoCosm (Joint Genome Institute), Phytozome (Joint Genome Institue), and RefSeq (an annotated set of sequences, including genomic DNA, transcripts, and proteins). In some instances, the genomic database may comprise a publicly-available database. In some instances, the genomic database may comprise a proprietary or private database.

In some instances, prior to performing the search candidate query (or target) genomes selected for a target agnostic or targeted search may be grouped into sets of two or more target genomes that, for example, maximize genomic diversity, focus the search on a given genera, or focus the search on closely related genomes. Non-limiting examples of the criteria that may be used to group two or more genomes include pairwise sequence identity, pairwise sequence similarity, and phylogenetic distances. In some instances, a group of query genomes may comprise 2, 4, 6, 8, 10, 20, 30, 40, 50, 60, 70, 80, 90, 100, or more than 100 genomes.

Pairwise sequence alignment methods are used to find the best pairwise (local or global) alignments of two query sequences. In some instances, pairwise sequence identity or sequence similarity may be calculated by comparing, e.g., single copy protein (amino acid) sequences or gene (nucleotide) sequences shared between the two genomes. Single copy proteins or genes can be annotated using tools such as BUSCO (Manni, et al. (2021), BUSCO Update: Novel and Streamlined Workflows along with Broader and Deeper Phylogenetic Coverage for Scoring of Eukaryotic, Prokaryotic, and Viral Genomes, Mol. Biol. Evol. 38(10):4647-4654), which estimates the completeness and redundancy of processed genomic data based on universal single-copy orthologs. In some instances, a pre-defined subset of proteins or genes may also be used. Selected proteins or genes are individually aligned, trimmed, and concatenated to calculate the pairwise sequence identity, i.e., the number of identical amino acid residues or nucleotides in the alignment. In some instances, pairwise sequence identity may be calculated by performing alignment of whole genomes and calculating the percent sequence identity between the pair of genomes.

Alternatively, a sequence similarity score can be calculated based on substitution matrices like BLOSUM (used to score local alignments between evolutionarily divergent protein sequences) and PAM (used to score global alignments between closely related protein sequences). See, e.g., Trivedi, et al. (2020), “Substitution Scoring Matrices for Proteins—An Overview”, Protein Science 29:2150-2163.

Genome grouping: In some instances, two or more target genomes may be grouped according to phylogenetic distance. Single-copy proteins or genes, or specific sequences such as the internal transcribed spacer (ITS) region (i.e., the DNA spacer situated between the small-subunit ribosomal RNA (rRNA) and large-subunit rRNA genes in the chromosome (or the corresponding transcribed region in the polycistronic rRNA precursor transcript), can be used to create phylogenetic trees from a set of genomes. The resulting phylogenetic trees are parsed to identify the phylogenetic distance (or degree of genomic divergence, as indicated by branch length) between all pairs of genomes.

In some instances, if a genome grouping value such as pairwise percent sequence identity score, pairwise sequence similarity score, or phylogenetic distance score is calculated, the target genomes may be grouped by first applying a specified threshold for the given grouping value to eliminate candidate target genomes (or pairs of candidate target genomes) that fail to meet the threshold, and then clustering the remaining target genomes into sets by using the Markov Cluster (MCL) algorithm or another clustering algorithm. In some instances, pairs of candidate target genomes may be retained and grouped if their pairwise percent identity, or pairwise sequence similarity, is greater than 50%, greater than 60%, greater than 70%, greater than 75%, greater than 80%, greater than 85%, greater than 90%, or greater than 95%. Examples of other clustering algorithms that may be used to group target genomes include, but are not limited to, a k means clustering method, a hierarchical clustering method, a mixture model method, or any combination thereof.

In some instances, taxonomic relationships from a known or curated database can be used to group genomes based on, e.g., Phylum, Class, Order, Family, Genus, or Species.

Target agnostic searches: At step 406 in FIG. 4, a target agnostic search is performed to determine the presence of absence of homologs of a plurality of query targets in the one or more selected target genomes. The results of the target agnostic search are then optionally dereplicated at step 410 by selecting one representative genome if multiple target genomes of the same species are identified as positive genomes.

Targeted searches: At step 408 in FIG. 4, a targeted search is performed to determine the presence of absence of homologs of the one or more query targets in the one or more selected target genomes. The results of the target agnostic search are then optionally dereplicated at step 412 by selecting one representative genome if multiple target genomes of the same species are identified as positive genomes.

For either target agnostic or targeted searches, the search may be performed using any of a variety of genomics search tools. Examples include, but are not limited to, BLAST, Diamond, HMMER (software for sequence alignment and searching sequence databases for homologs using probabilistic models called profile hidden Markov models (pHMMs)), or ggsearch.

For either target agnostic or targeted searches, the search can be performed across the entire genome of the one or more selected target genomes or it may be restricted to specific locations or regions (e.g., promoter regions, coding regions, introns, exons, termination sequences, etc.) of the one or more selected target genomes. In some instances, the search may be restricted to regions of the one or more selected target genomes that are known to be, or are predicted to be, biosynthetic gene clusters (BGCs). In some instances, BGCs may be predicted by sequence analysis tools such as antiSMASH (software for identifying, annotating, and comparing gene clusters that encode the biosynthesis of secondary metabolites in bacterial and fungal genomes; see, e.g., Medema, et al. (2011), “antiSMASH: Rapid Identification, Annotation and Analysis of Secondary Metabolite Biosynthesis Gene Clusters in Bacterial and Fungal Genome Sequences”, Nucleic Acids Research 39, web server issue W339-W346), SMURF (web-based software for predicting clustered secondary metabolite genes based on their genomic context and domain content; see, e.g., Khaldi, et al. (2010), “SMURF: Genomic Mapping of Fungal Secondary Metabolite Clusters”, Fungal Genet Biol. 47(9):736-741), TOUCAN (supervised learning framework for identifying fungal biosynthetic gene clusters based on protein or nucleotide sequences); see, e.g., Almeida, et al. (2020), “TOUCAN: a framework for fungal biosynthetic gene cluster discovery”, NAR Genomics and Bioinformatics 2(4):1gaa098, Deep-BGC (deep learning-based software for biosynthetic gene cluster prediction; see, e.g., Hannigan, et al. (2019), “A Deep Learning Genome-Mining Strategy for Biosynthetic Gene Cluster Prediction”, Nucleic Acids Research 47(18):e110), or custom search algorithms.

BGC prediction: BGCs can be predicted by extracting regions (of a specified length) from target genomes that are adjacent to gene sequences that appear to be homologs of known biosynthetic core synthase genes based on sequence searches (using tools such as BLAST, Diamond, or ggsearch), HMMs of known core synthases (using tools such as HMMER), or co-localization of protein domains associated with core synthases. Candidate putative embedded target genes (pETaGs) are identified if matches to the target gene sequence are found within predicted BGCs or within specified sequence regions of the target genome(s). A search result is considered a match to a query target if the search result exceeds a specified threshold for sequence similarity according to any of a variety of metrics known to those of skill in the art. Examples include, but are not limited to, percent sequence identity (e.g., at least 20%, 30%, 40%, 50%, 60%, 70%, 80%, 85%, 90%, 95%, 96%, 97%, 98%, 99%, or higher), percent sequence coverage (e.g., at least 20%, 30%, 40%, 50%, 60%, 70%, 80%, 85%, 90%, 95%, 96%, 97%, 98%, 99%, or higher)), E-value (e.g., 10, 1, 0.1, 0.001, 0.0001, 1e⁻¹⁰, 1e⁻²⁰, 1e⁻¹⁰⁰, or lower), bitscore (e.g., 5, 10, 25, 50, 100, 250, 500, 1000, 5000 or more), or HMM score (e.g., 5, 10, 25, 50, 100, 250, 500, 1000, 5000 or more).

Sensitive targeted search techniques: In some instances of a targeted search, an additional search method can be utilized to increase the sensitivity of the search and capture hard-to-find signals of pETaG presence. Searches for query targets comprising protein sequences or nucleotide sequences, or pHMMs of the query targets, are often performed using sequence alignment tools such as BLAST, Exonerate, HMMER, etc. If the query targets are protein sequences, protein-DNA search tools such as TBLASTN or Exonerate may be used to convert protein sequence targets to nucleotide sequences, and then perform a search of the nucleotide sequence against the one or more query genomes (or target genomes). Alternatively, probabilistic models such as PFAM or TIGRFAM can also be used with an HMM search tool such as HMMER. The resulting search hits can be filtered by genome location and/or by comparison of sequence metrics such as E-value, percent sequence identity, query coverage (e.g., the degree of coverage of the query sequence by the identified homologous sequence), subject coverage (e.g., the degree of overage of the identified homologous sequence by the query sequence of interest), or bitscore, and the like to a corresponding cutoff threshold. Cutoff threshold for percent sequence identity may be at least 20%, 30%, 40%, 50%, 60%, 70%, 80%, 85%, 90%, 95%, 96%, 97%, 98%, 99%, or higher. Cutoff thresholds for subject coverage and/or query coverage may be at least 20%, 30%, 40%, 50%, 60%, 70%, 80%, 85%, 90%, 95%, 96%, 97%, 98%, 99%, or higher. Cutoff thresholds for E-value may be 10, 1, 0.1, 0.001, 0.0001, 1e⁻¹⁰, 1e⁻²⁰, 1e⁻¹⁰⁰, or lower. Cutoff thresholds for bitscore may be 5, 10, 25, 50, 100, 250, 500, 1000, 5000 or more.

DNA alignment regions from the search results may be evaluated by comparing the genomic coordinates of the alignment regions with given protein sequence predictions in the query genome. If the DNA alignment region overlaps a single predicted protein sequence, and a corresponding sequence similarity metric (such as E-value, percent sequence identity, query coverage, subject coverage, or bitscore) for the DNA alignment region and the predicted protein sequence exceeds a specified threshold for the sequence similarity metric (as described above), the corresponding nucleic acid sequence is reported as a pETaG (Region Case 1).

If the DNA alignment region overlaps a plurality of predicted protein sequences, and a corresponding sequence similarity metric (such as E-value, percent sequence identity, query coverage, subject coverage, or bitscore) for the DNA alignment region and each of the plurality of predicted protein sequences exceeds a specified threshold, only one of the predicted protein sequences (or its corresponding nucleic acid sequence) is reported as the pETaG (Region Case 2). In some instances, the decision of which overlapping protein sequence to report as the pETaG can be based on which has the highest corresponding sequence metric (i.e., the protein sequence (or its corresponding nucleic acid sequence) for which the E-value, percent sequence identity, query coverage, subject coverage, or bitscore has the highest value is reported as the pETaG). In some instances, the decision of which overlapping protein sequence to report as the pETaG can be based on protein sequence length (i.e., the longest overlapping protein sequence (or its corresponding nucleic acid sequence) is reported as the pETaG).

If the DNA alignment region is overlapping a single predicted protein sequence or multiple predicted protein sequences, but none of the corresponding sequence similarity metrics exceed a specified threshold, the longest protein sequence or the protein sequence having the highest corresponding sequence similarity metric value is reported as a pETaG (Region Case 2).

If the DNA alignment region does not overlap a predicted protein sequence, the coordinates of the DNA alignment region are reported as a pETaG (Region Case 3).

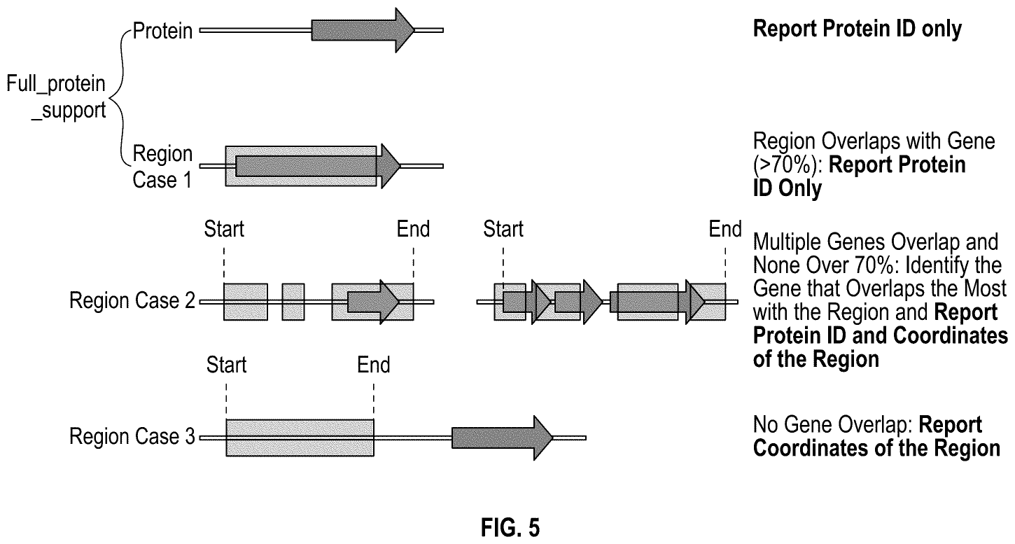

Review of targeted search results and the reporting of pETaGs: FIG. 5 illustrates the different cases described for targeted search results. The protein case illustrated at the top of the figure depicts the scenario where the targeted search method (e.g., a protein search, a HMM search, etc.) identifies a full protein in the target genome of interest as a direct hit for the search query (e.g., a human gene), and the protein ID is reported as corresponding to a pETaG. Region Case 1 illustrates the scenario where a DNA sequence search identifies a DNA alignment region that overlaps part of a predicted protein sequence with high sequence similarity (e.g., >70% sequence identity in this example), and the protein ID is reported as corresponding to a pETaG. Region Case 2 illustrates the scenario where a DNA sequence search identifies DNA alignment region(s) that overlap part of a predicted gene sequence, extensions of a predicted gene sequence, or multiple predicted gene sequences, with none of the overlapping sequences exhibiting a sequence similarity metric that exceeds a specified threshold value (e.g., >70% sequence identity), and the protein ID for the protein sequence that exhibits the largest degree of overlap and the coordinates of the DNA alignment region are reported as a pETaG. Region Case 3 illustrates the scenario where a DNA sequence search identifies a DNA alignment region that does not overlap a predicted protein sequence in the target genome of interest, and the coordinates of the DNA alignment region are reported as a pETaG.

Generating comparative genomics heatmaps: Returning to FIG. 4, at step 414 comparative genomics heatmaps are generated for each pETaG identified by the target agnostic or targeted search, and then used to evaluate the pETaG. The heatmaps, or the underlying data from the heatmaps, may be used, for example, to evaluate the degree of “embeddedness” (i.e., the degree of association with a BGC) of a given pETaG. In some instances, vector representations of the data included in a heat map may be provided to one or more trained neural networks (e.g., trained long short-term memory (LSTM) models) to perform an embeddedness classification of the corresponding pETaG. Methods for generating comparative genomics heatmaps and using them to evaluate pETaGs will be described in more detail below.

Calculation of grouped genome-derived metrics: At step 416 in FIG. 4, grouped genome-derived metrics are calculated if two or more target genomes are optionally grouped into sets. The grouped genome-derived metrics may then be used to evaluate pETaGs. In some instances, target genomes may be grouped prior to performing query searches, as described above. In some instances, target genomes may be grouped after performing query searches of all target genomes for the query targets of interest. For each set of target genomes and for each pETaG within the set of genomes, several search features can be computed. Examples of grouped genome-derived metrics (or search features) include, but are not limited to, (i) the number of “positive” genomes in the genome set (i.e., the number of genomes that contain the core synthase, where the BGC has been predicted by using, e.g., antiSMASH, SMURF, TOUCAN, deepBGC, or a custom BGC identification method), (ii) the number of “negative” genomes in the genome set (i.e., the number of genomes that do not contain the core synthase), and (iii) a copy number difference (CND) for a given gene between positive and negative genomes (an indicator of a gene's involvement in a resistance mechanism is duplication, so pETaGs are evaluated as being an extra copy of, e.g., a housekeeping gene). In some instances, a positive genome can be defined as a genome that contains a candidate BGC, independent of a core synthase gene. In some instances, a negative genome can be defined as a genome that does not contain a candidate BGC, independent of a core synthase gene.

Clusters of orthologous groups (COGs): In some instances, Clusters of Orthologous Groups (COGs) of proteins or genes may be used to determine copy number differences (“COG CNDs”). COGs can be faster to generate and provide a somewhat orthogonal/alternative way to calculate copy number differences than tree-based approaches. COGs may be identified by performing an all-versus-all protein (amino acid) sequence search using all of the target genomes in a given genome set using tools such as BLASTp, ggsearch, or Diamond-BLASTp. Members of the same COG are presumed to have orthologous functions. COGs can also be defined as gene or protein families (or orthogroups, i.e., sets of genes or proteins derived from a single gene in the last common ancestor of the set of target species/genomes under consideration). COGs may also be identified using tools such as OrthoMCL, OrthoFinder, PanX, or other orthogroup/pangenome creation tools, or using protein clustering tools such as USEARCH, CD-HIT, and MMseqs. Pairwise associations are established between each query target and corresponding target protein sequence identified in the search based on, for example, either the percent sequence identity or E-value, and are then clustered using MCL, SiLiX, or other clustering algorithms. For COGs of interest, the copy number difference is calculated by subtracting the average number of target gene homologs in negative genomes from the average number of target gene homologs in positive genomes present within the COG.

Agnostic tree CNDs: In some instances, copy number differences may be calculated from a phylogenetic tree created for the set of grouped target genomes (i.e., “agnostic tree CNDs”). COGs are identified as described above or, in some instances, the same COGs used to determine COG CNDs may also be used to determine agnostic tree CNDs. For the COG that contains the pETaGs, a multiple sequence alignment is created using any multiple sequence alignment software tool (e.g., MAFFT, MUSCLE, ClustalW, etc.) and then trimmed using any sequence trimming software tool (e.g., trimAI, GBlocks, ClipKIT, etc.). The resulting trimmed sequence alignment is then used to create a phylogenetic tree using any phylogenetic tree reconstruction software (e.g., FastTree, IQ-TREE, RAxML, MEGA, MrBayes, BEAST, PAUP, etc.). Additionally, phylogenetic trees can also be constructed using various algorithms, such as maximum likelihood algorithms, parsimony algorithms, neighbor joining algorithms, distance matrix algorithms, Bayesian inference algorithms, or any combination thereof. The resulting phylogenetic tree is then computationally parsed to identify the last common ancestor clade of the pETaG and housekeeping versions of the pETaG. The last common ancestor may be determined by first identifying the clade that contains all or defined subset of the pETaGs from positive genomes (i.e., the pETaG clade). From the pETaG clade, the phylogenetic tree may then be traversed by walking backwards towards the root and checking if each new clade contains genes from all or a defined subset of negative genomes. Once the correct clade is identified (i.e., the clade that contains all or defined subset of the pETaGs and the genes from all or a defined subset of negatives genomes), it is identified as the last common ancestor clade. The agnostic tree CND is then calculated for a given pETaG by subtracting the average number of gene homologs in the negative genomes from the average number of gene homologs in the positive genomes present within the last common ancestor clade.

Evolutionary metrics: In some instances, candidate pETaGs may also be evaluated based on one or more evolutionary metrics (e.g., phylogenetic features, co-occurrence features, or co-evolution features) determined at step 416 in FIG. 4 using a custom software algorithm that utilizes a set of positive genomes and negative genomes to evaluate whether a candidate pETaG is associated with a candidate core synthase or BGC. Positive genomes are defined as genomes that contain a candidate core synthase in a candidate BGC and at least one of the positive genomes contains the candidate pETaG. In some embodiments, a positive genome can be defined as genomes that contain a candidate BGC, independent of a core synthase gene. Negative genomes are defined as genomes that do not contain candidate core synthase in a candidate BGC. In some embodiments, a negative genome can be defined as genomes that do not contain a candidate BGC, independent of a core synthase gene. The positive and negatives genomes used to calculate evolutionary metrics can be identified using any of a variety of methods known to those of skill in the art. For example, the comparative genomics heatmaps, or the underlying data thereof, described elsewhere herein can be supplied to the custom software algorithm. Alternatively, any method that utilizes protein or DNA sequence searches to determine if a query sequence of interest is co-located with a core synthase and/or is located within a predicted BGC may be used.

To evaluate evolutionary metrics for a candidate pETaG, a minimum number of positive and or negative genomes are required to perform certain analyses. For example, phylogenetic features require at least 1 positive genome and at least 1 negative genome for analysis. Co-occurrence features require at least 1 positive genome and at least 1 negative genome. Co-evolution features require at least 3 positive genomes, but there is no requirement for identifying a negative genome. The actual number of positive and negatives genomes used can be any number greater than the required minimum number for a given analysis. In some instances, the number of positive genomes and/or negative genomes used in evaluating evolutionary metrics may be at least 1, 2, 3, 4, 5, 6, 7, 8, 9, 10, 20, 30, 40, 50, 60, or more than 60 genomes. In some instances, the number of combined positive genomes and/or negative genomes used does not exceed 20 genomes in order to preserve computational efficiency.

In some instances, the positive genomes may optionally be dereplicated by species (i.e., filtered so that only one representative genome is kept if multiple genomes of the same species are identified as positive genomes). Dereplication of the genomes can be performed in several ways, e.g., based on taxonomy if species names are known, based on pairwise sequence identity or similarity as described above for the search methodology (a species-level distinction can be determined based on a specified threshold for the pairwise sequence identity (e.g., 99.9%, 99.8%, 99.7%, 99.6%, 99.5%, 99.4%, 99.3%, 99.2%, 99.1%, 99%, 98%, 97%, 96%, 95%, 94%, 93%, 92%, 91%, 90%, 85%, 80%, or lower) or similarity (threshold values will vary depending on sequence length, number of sequences used, and the similarity matrix that is used)), or based on phylogenetic distance as described above for the search methodology (a species-level distinction can be determined based on a specified threshold for the pairwise phylogenetic distance (e.g., 0.0001, 0.001, 0.002, 0.003, 0.004, 0.005, 0.006, 0.007, 0.008, 0.009, 0.01, 0.02, 0.03, 0.04, 0.05, 0.1, or higher)). In some embodiments, the positive genomes may optionally be dereplicated by other taxonomic rankings such as, but not limited to genera, family, order, class, or phylum.

In some instances, the genomes selected for use as negative genomes may be those genomes that are closest to positive genomes but classified as being negative. The closeness of a negative genome to a positive genome can be determined in any of several ways, e.g., based on taxonomy if species names are known, based on pairwise sequence identity or similarity as described above for the search methodology (e.g., negative genomes are selected that have the highest percent identity or similarity to the positive genomes), or based on phylogenetic distance as described above for the search methodology (e.g., negative genomes are selected that have the lowest phylogenetic distance to the positive genomes).

Phylogenetic features: Phylogenetic features may be calculated from a phylogenetic tree of the last common ancestor clade of the candidate pETaG and its housekeeping copy. The process of creating the phylogenetic tree and identifying the last common ancestor is described as follows. In a first step of the process, the pETaG is used as a query sequence to identify all homologs in the set of selected positive and negative genomes using any sequence search and alignment tool (e.g., BLASTp, ggsearch, or Diamond-BLASTp, etc.). Then, the homolog sequences are aligned using any alignment software tool (e.g., MAFFT, MUSCLE, ClustalW, etc.) and trimmed using any sequence trimming software (e.g., trimAI, GBlocks, ClipKIT, etc.). The resulting trimmed sequence alignment is then used to create a phylogenetic tree using any phylogenetic reconstruction software tool (e.g., FastTree, IQ-TREE, RAxML, MEGA, MrBayes, BEAST, PAUP, etc.). Alternatively, phylogenetic trees can be constructed using various algorithms, such as maximum likelihood algorithms, parsimony algorithms, neighbor joining algorithms, distance matrix algorithms, Bayesian inference algorithms, or any combination thereof. The resulting phylogenetic tree is then computationally parsed to identify the last common ancestor clade of the pETaG and the house keeping versions of the pETaG. The last common ancestor is determined by first identifying the clade that contains all or subset of the pETaGs from positive genomes (i.e., the pETaG clade). From the pETaG clade, the phylogenetic tree may then be traversed by walking backwards towards the root and checking if each new clade contains genes from all or a subset of negative genomes. Once the correct clade is found (i.e., the clade that contains all or subset of pETaGs and the genes from all or a defined subset of negatives genomes), it is identified as the last common ancestor clade (LCA).

The last common ancestor (LCA) clade may be used to calculate a variety of metrics from the phylogenetic tree. Examples include, but are not limited to:

Phylogenetic tree CND—calculated for a given gene (e.g., pETaG) by subtracting the average number of gene copies in the negative genomes from the average number of gene copies in the positive genomes present within the last common ancestor clade.

Ratio mean to LCA—calculated as the average branch length for all pETaG genes to the LCA node divided by the average branch length for all house copy genes to the LCA node.

Ratio standard deviation (stdev) to LCA—calculated as the standard deviation of the branch lengths for all pETaG genes to the LCA node divided by the standard deviation of the branch lengths for all house copy genes to the LCA node.

Ratio mean neighbor distance—calculated as the average branch length for each pETaG gene to all other pETaGs divided by the average branch length for each house copy gene to all other house copy genes.

Ratio standard deviation (stdev) neighbor distance—calculated as the standard deviation of branch lengths for each pETaG gene to all other pETaG is divided by the standard deviation of branch lengths for each house copy gene to all other house copy genes.

Ratio clade sum—calculated as the sum of all branch lengths in the pETaG clade (i.e., the clade in the phylogenetic tree that contains all or subset of pETaGs) divided by the sum of all branch lengths in the house copy clade (i.e., the clade in the phylogenetic tree that contains all or subset of house copy genes).

Alternative phylogenetic metrics: In some cases, alternative metrics may be calculated from the phylogenetic tree and used as additional evidence for a high probability that a pETaG is an actual ETaG. Examples include, but are not limited to, the Robinson-Foulds (RF) distance metric, which is used to compute the distance between two phylogenetic trees (in this case, the distance between the pETaG clade and the House copy clade). Similar RF distance metrics or reconciliation-based distances metrics can be computed using tools such as TreeKO to compare the topology and distances of the pETaG and house copy clade.

Co-occurrence features: Co-occurrence features may be calculated from clusters of orthologous groups (COGs) of protein or gene sequences. COGs may be identified by performing an all-versus-all protein (amino acids) sequence search or an all-versus-all nucleotide sequence search, using all positive and negative genomes, using software tools such as BLAST, ggsearch, or Diamond. Reciprocal best-hits (i.e., where the best protein match between genome A and genome B must also be the best protein match between genome B and genome A) are used to establish an association between protein sequences (or their nucleotide sequence counterparts), which are then clustered using, e.g., MCL, SiLiX, or other clustering algorithms. Alternatively, in some instances, unidirectional search results may be used (instead of reciprocal searches) to create associations prior to clustering. COGs may also be identified using tools such as OrthoMCL, OrthoFinder, PanX, or other orthogroup/pangenome creation tools, or using protein clustering tools such as USEARCH, CD-HIT, and MMseqs.

A variety of co-occurrence metrics may be calculated from the identified COGs. Examples include, but are not limited to:

Normalized co-occurrence distance metric: A normalized co-occurrence distance metric is calculated for all COGs based on the following formula:

$$Normalized Distance : \frac{\sqrt{{( TPG - PG )}^{2} + {( 0 - NG )}^{2}}}{\sqrt{{( TPG )}^{2} + {( TNG )}^{2}}}$$

where TPG is the total number of positive genomes, TNG is the total number of negative genomes, PG is the number of positive genomes containing genes in the given COG, and NG is the number of negative genomes containing genes in the given COG.

Co-occurrence pETaG distance—calculated as the distance score of the COG that contains the pETaG.

Co-occurrence pETaG rank—the rank of the distance score of the COG that contains the pETaG relative to the distance scores for other COGs, in ascending order. In the case of ties for a distance score, the rank assigned for all COGs in the tie is the lowest rank in the group.

Co-occurrence core distance—the distance score of the COG that contains the core synthase.

Co-occurrence core rank—the rank of the distance score of the COG that contains the core synthase relative to the distance scores for other COGs, in ascending order. In the case of ties for a distance score, the rank assigned for all COGs in the tie is the lowest rank in the group.

Co-evolution features: Co-evolution features may also be calculated from clusters of orthologous groups (COGs) of protein or gene sequences. COGs may be identified in a similar manner to that described above, e.g., by performing an all-versus-all protein (amino acid) sequence search using all of the target genomes in a given genome set using tools such as BLASTp, ggsearch, or Diamond-BLASTp and subsequent clustering of the pairwise sequence similarity results. Only COGs that include at least 3 genes that are single-copy genes are passed to the co-evolution analysis. Single-copy means that COG consists of only 1 gene/protein per genome, of genomes present in the COG.

Co-evolution analysis comprises the determination of COG-to-COG comparison values, which can be done using several different approaches, e.g., multiple sequence alignments (MSAs) or gene-tree comparisons.

Multiple sequence alignments (MSAs) can be created for each of a pair of COGs using any of a variety of alignment software tools including, but not limited to, MAFFT, MUSCLE, and ClustalW. MSAs can then be trimmed based on specified parameters (such as removal of all gaps, removal of gaps such that the number of sequential gaps are greater than a specified threshold, keeping all gaps, etc.), and a percent sequence identity (the number of identical residues in the alignment) is calculated. Alternatively, a sequence similarity score can be calculated based on substitution matrices like BLOSUM and PAM.

Gene-tree comparisons can be calculated based on phylogenetic trees generated from amino acid or nucleotide sequences within each of a pair of COGs by sequence alignment, trimming, and phylogenetic reconstruction. Phylogenetic trees must be constrained to the topology of the species tree of the target genomes, with genes present in both COGs being compared. Pairwise branch lengths between the two resulting COG trees are then calculated.

This analysis can be performed using a custom script or using software tools such as the Co-Variance algorithm in PhyKIT (https://github.com/JLSteenwyk/PhyKIT).

Either percent sequence identities, sequence similarities, or branch length comparisons are then used to calculate the correlation of all pairwise COG combinations using Pearson R, or any other correlation metric known to one of skill in the art. Correlations can only be computed between pairs of COGs that share at least 3 genomes.

Examples of the co-evolution metrics that may be calculated include, but are not limited to:

Co-evolution correlation—the correlation of the pairwise percent sequence identities of COG_{X} with the pairwise percent sequence identities of COG_{Y}.

Co-evolution rank—the rank of the correlation of COGₓ with COG_{y}, relative to all other pairwise COG correlations in ascending order. In the case of ties, the rank assigned to all pairwise COG correlations in the tie is the lowest rank in the group.

Co-evolution slope—the orthogonal regression of the pairwise percent identities of COG_{X} with the pairwise percent identities of COG_{Y}.

In some instances, COGₓ is the COG that contains the core synthase and COG_{y} is the COG that contains the pETaG.

Genome data set features: A variety of genome data set features may be calculated for groups of target genomes that have been clustered as described above. Examples include, but are not limited to:

Number positive genomes—the final number of positive genomes that have been used in any and all metrics described above. In some instances, 10, 15, 20, 25, 30, or more positive genomes may have been used as input.

PGI mean positives—the mean pairwise genomic identity (PGI) between the final number of positive genomes. The pairwise genomic identity may be calculated, e.g., by comparing, e.g., single copy protein (amino acid) sequences or gene (nucleotide) sequences shared between the two genomes.

PGI standard deviation (stdev) positives—the standard deviation of the pairwise genomic identity between the final number of positive genomes.

Number negative genomes—the final number of negative genomes that have been used in any and all metrics described above. In some instances, 10, 15, 20, 25, 30, or more negative genomes may have been used as an input.

PGI mean negatives—the mean pairwise genomic identity between the final number of negative genomes.

PGI standard deviation (stdev) negatives—the standard deviation of the pairwise genomic identity between the final number of negative genomes.

Returning to FIG. 4, at step 418 the comparative genomics heatmaps, or the underlying data thereof, that were generated for each pETaG identified in the search may be used to evaluate the degree of “embeddedness” of a given pETaG, as noted above. In some instances, vector representations of the data included in a heat map may be provided to one or more trained neural networks (e.g., trained long short-term memory (LSTM) models, convolutional neural networks (CNNs)) to perform an embeddedness classification of the corresponding pETaG. For example, a comparative genomics heatmap, or the underlying data therefrom, may be analyzed using a trained LSTM model to predict the probability that a pETaG is associated with a BGC. Methods for generating comparative genomics heatmaps and using them to evaluate pETaGs will be described in more detail below.

At step 420 in FIG. 4, a “feature table” is compiled that summarizes the grouped genome-derived metrics and/or embeddedness classification data for the pETAGs identified in the search. In some instances, the feature table, or data included therein, may be used as input for a machine learning-based analysis (e.g., a deep learning-based analysis) to evaluate pETaGs.

Machine learning-based (e.g., deep learning-based) evaluation of pETaGs: At step 422 in FIG. 4, a machine learning-based analysis (e.g., a deep learning-based analysis) may be performed to evaluate the likelihood (or probability) that a given pETaG is an actual ETaG. The input for the machine learning-based analysis is the feature table, or data included therein, compiled at step 420 in FIG. 4. In some instances, predicted BGC regions for the target genomes, identified using a BGC search tool as indicated at step 424, may also be used as input for the machine learning-based evaluation. As indicated, in some instances the predicted BGC regions for target genomes may also be used as part of the selection of target genomes to be used in the search (e.g., at step 404 in FIG. 4). As noted above, BGCs can be predicted by extracting regions (of a specified length) from target genomes that are adjacent to gene sequences that appear to be homologs of known biosynthetic core synthase genes based on sequence searches (using tools such as BLAST, Diamond, or ggsearch), HMMs of known core synthases (using tools such as HMMER), or co-localization of protein domains associated with core synthases.

Any of a variety of machine learning algorithms may be used in implementing the disclosed pETaG evaluation methods. For example, the machine learning algorithm employed may comprise a supervised learning algorithm (e.g., an algorithm that relies on the use of a set of labeled training data to infer the relationship between a set of one or more pETaG features and a prediction of the probability that a given pETaG is an actual ETaG), an unsupervised learning algorithm (e.g., an algorithm used to draw inferences from training datasets consisting of pETaG features that are not paired with labeled ETaG classification or probability data), a semi-supervised learning algorithm (e.g., an algorithm that makes use of both labeled and unlabeled pETaG feature data for training (typically using a relatively small amount of labeled data with a larger amount of unlabeled data), a decision tree model (e.g., an algorithm that models the learning task as a series of individual decisions, and specifically, gradient boosted trees, in which new trees are created to model errors or residuals to supplement and enhance existing trees that may be used to partition or classify pETaGs based on feature data), a deep learning algorithm (e.g., an algorithm inspired by the structure and function of the human brain such as an artificial neural network (ANN), and specifically, a large neural network comprising many hidden layers of coupled “nodes” that may be used to map pETaG feature data to probability predictions or classification decisions), or any combination thereof.

A deep learning model (i.e., a trained deep learning algorithm) may comprise any total number of layers, and any number of hidden layers, where the hidden layers function as trainable feature extractors that allow mapping of a set of input data to a preferred output value or set of output values. Each layer of the neural network comprises a number of nodes (or units). A node receives input that comes either directly from the input data (e.g., pETaG feature data derived using the methods described above) or the output of nodes in previous layers, and performs a specific operation, e.g., a summation operation. In some cases, a connection from an input to a node is associated with a weight (or weighting factor). In some cases, the node may, for example, sum up the products of all pairs of inputs, Xᵢ, and their associated weights, Wᵢ. In some cases, the weighted sum is offset with a bias, b. In some cases, the output of a node may be gated using, e.g., a threshold or activation function, f, which may be a linear or non-linear function. The activation function may be, for example, a rectified linear unit (ReLU) activation function or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, can be “taught” or “learned” in a training phase using one or more sets of training data. For example, the parameters may be trained using the input data from a training data set and a gradient descent or backward propagation method so that the output value(s) (e.g., a prediction of the probability that a given pETaG is an actual ETaG) that the deep learning model computes are consistent with the examples included in the training data set.

For example, in some instances, a deep learning model trained for predicting a probability that a given pETaG is an actual ETaG may comprise a fully connected neural network having a specified number of hidden layers (e.g. 2, 4, 6, 8, 10, 12, 14, 16, 18, 20, 100, 1000 or more than 1000 hidden layers) and a specified number of units per hidden layer (e.g., 1, 2, 4, 8, 16, 32, 64, 126, 256, or more than 256 units per hidden layer). The output layer may comprise a single unit configured to predict the similarity of a given pETaG to the known ETaGs used for training the model (e.g., the model is trained to predict a probability that the given pETaG is an actual ETaG). In the case of supervised learning, for example, the training data set may comprise feature data for a set of known (or positive) ETaGs to create a set of true positives. The training data set may also comprise feature data for a set of negative ETaGs to create a set of true negatives (i.e., negative ETaGs are gene or protein sequences that are not ETaGs). Known examples of positive and negative ETaGs may be identified, e.g., from the scientific literature and/or through in-house research. Examples of pETaG feature data that may be used for training the model (or for performing an empirical evaluation of pETaGs, as described below) are summarized in Table 1.

TABLE 1
Metrics (features) for Deep Learning-Based and Empirical
Evaluation of pETaGs

| | |
|---|---|
| Number of pETaG-core genomes | Co-occurrence pETaG distance |
| Number of non-pETaG-core | Co-occurrence pETaG rank |
| genomes | |
| ETaG COG CND | Co-occurrence core distance |
| Agnostic tree CND | Co-occurrence core rank |
| Tier A+ (heatmap probability) | Co-evolution correlation |
| Tier 1 (heatmap probability) | Co-evolution rank |
| Tier 2 (heatmap probability) | Co-evolution slope |
| Tier 3 (heatmap probability) | Number of positive genomes |
| Phylogenetic tree CND | PGI mean positives |
| Ratio mean LCA | PGI stdev positives |
| Ratio stdev LCA | Number negative genomes |
| Ratio mean neighbor distance | PGI mean negatives |
| Ratio stdev neighbor distance | PGI stdev negatives |
| Ratio clade sum | |

In some instances, the training data comprising true positives and true negatives may be split into training-test data sets (e.g., using a split of 90/10, 80/20, 70/30, etc.) and first used to train the deep learning model and then test the deep learning model. In some instances, a positive or negative weight can be used to balance the training data set of true positives and true negatives if the initial data set is unbalanced.

In one non-limiting example, a deep learning model for predicting a probability that a given pETaG is an actual ETaG was trained using a stochastic gradient descent algorithm and data from an initial data set training-test split of 80/20 using the following training parameters:
Gradient clip max norm=1.0 (i.e., the gradient was “clipped” if its normalized value exceeded a maximum value of 1.0)
Mini batch size=512 (i.e., 512 training data pairs were used per training iteration)
Learning rate=1e-5 (i.e., the weights were updated by an incremental value of 1e-5 in each training iteration)
Max epochs=3,000 (i.e., the number of complete passes through the training data set during training)
Number of hidden layers: models comprising varying numbers of hidden layers were used and later evaluated for performance
Number of units per hidden layer: models comprising varying numbers of units per hidden layer were used and later evaluated for performance

One the deep learning model has been trained, it may be tested using, e.g., the test dataset from the initial training-test data set split. An example of test results from a model with two hidden layers and varying number of units per hidden layer is presented below.

Use of empirical scores to evaluate pETaGs: In some instances, an empirical scoring method may be used instead of, or in addition to, a deep learning-based method for evaluating pETaGs. As with the deep learning-based approach, the analysis is performed using a pETaG data set comprising examples of data for positive (known) ETaGS and negative (known not to be) ETaGs. A set of positive ETaGs can be identified to create a set of true positives. A set of negative ETaGs (i.e., gene or protein sequence that are known not to be ETaGs) can be identified to create a set of true negatives. Positive and negative ETaGs may be identified from, e.g., the scientific literature and/or through in-house research. Candidate pETaGs selected from the positive and negative ETaG data set may be analyzed using the search methods described above (e.g., the target agnostic search methods) and evaluated using a specified combination of the metrics described above.

The results from the analysis of positive and negatives ETaGs may be used to determine a set of rules, weights, scores, and/or thresholds based on the values of the different metrics summarized in Table 1. The resulting set of rules, weights, scores, and/or thresholds may then be used to develop an algorithm for filtering the pETaG results and classifying candidate pETaGs as positive or negative ETaGs.

The set of rules, weights, scores, and/or thresholds assigned to the different metrics may be determined based on an analysis to identify if the corresponding values for a candidate pETaG are more similar to those for positive ETaGs or to those for negative ETaGs. Decision points and metric value thresholds can be based, e.g., on commonly used statistical measurements and analyses including, but not limited to, mean, median, standard deviation, standard error, quartiles, confidence intervals, bootstrapping (or any variation thereof), jackknifing (or any variation thereof), or any combination thereof.

Algorithm testing may comprise testing all combinations of metric values for a positive and negative ETaG data set and corresponding rules, weights, scores, and/or thresholds for their performance in predicting the likelihood that a pETaG is an actual ETaGs. In some instances, a best algorithm may be selected based on a given target statistic to be maximized. For example, a first empirical model may be selected if one seeks to maximize precision, a second empirical model may be selected if one seeks to maximize sensitivity, and a third empirical model may be selected if one seeks to maximize, e.g., F1 score (a statistical measure of a test's accuracy). The selected model can then be used to analyze the results from a target evaluation, e.g., for a given candidate pETaG, and provide an empirical score which can be used to evaluate the likelihood or probability (and/or confidence level) that the given pETaG is a true ETaG.

Returning to FIG. 4, at step 426 a target look-up table is compiled based on deep learning-based and/or empirical pETaG evaluation methods that comprises an array of data that maps input values or ranges for the evolutionary metrics for pETaGs to output values for the probability that the pETaG is an actual ETaG.

### Grid Representation (Heatmap) Analysis Methods

In some instances, the methods and systems described herein relate to identification of genes associated with a gene cluster, e.g., a biosynthetic gene cluster (BGC), using machine-learning algorithms to assess grid representations (e.g., heat maps, or data matrices) of orthologs of genes that co-localize with an anchor gene (e.g., a core synthase gene) of the gene cluster, e.g., BGC, across diverse genomes.

The most commonly used bioinformatics tool to identify biosynthetic gene clusters is antiSMASH, which annotates more than 40 types of BGCs based on the presence of certain key protein domains. Currently, there is no good method available to accurately predict the genomic boundaries of a gene cluster, e.g., BGC. antiSMASH defines a BGC region using the following algorithm. In the first step, all gene products of the analyzed sequence are searched against a database of hidden Markov model (HMM) profiles for highly conserved enzymes (e.g., core-enzymes), which are indicative of a specific BGC type. In a second step, pre-defined cluster rules are employed to define individual “Clusters” encoded in the analyzed sequence regions. Each identified cluster includes core gene-product(s), or core synthase gene(s), that trigger the cluster rule. antiSMASH defines a BGC region by extending to the upstream and downstream of the core synthase gene(s) by a predetermined length, such as 20 kb. The predetermined lengths for the different cluster types are empirically determined and generally tend to over-include adjacent genes as part of a BGC. See, e.g., Blink K. et al., (2017) Nucleic Acids Res., 45, W36-W41; and Weber T. et al., antiSMASH5, antiSMASH Database Manual (2019). Therefore, genes identified as part of a BGC using antiSMASH or based on proximity to a core synthase gene of a BGC may not be functionally related to the secondary metabolite produced by the BGC.

To solve this problem, the methods and systems described herein leverage comparative genomics and machine-learning algorithms to assess heat maps representing distribution of orthologs, e.g., bidirectional best hits (BBHs), of genes co-localized with an anchor gene (e.g., a core synthase gene) known to associate with a BGC across a number of diverse genomes in order to determine a likelihood that an ortholog of a query gene (e.g., a gene in a reference genome) that is co-localized with an anchor gene (e.g., a core synthase gene) of a gene cluster (e.g., a BGC) in a query (or target) genome is associated with the BGC in the query genome. The machine-learning models can be trained using manually curated heat maps, including manually curated heat maps that represent co-localized non-biosynthetic genes that are known or experimentally verified to associate with gene clusters (such as BGCs), co-localized non-biosynthetic genes that are known or experimentally verified to have no functional association with gene clusters (such as BGCs), and genes that comprise borderline cases. The trained machine-learning algorithms allow rapid assessment of a large number of putative embedded genes that are co-localized with anchor genes (e.g., core synthase genes) in gene clusters such as BGCs using sequence information from a large number of genomes, which greatly improves the accuracy of delineating the borders of the gene cluster. Furthermore, the methods allow prioritization of putative embedded genes in gene clusters such as BGCs for downstream assessment, including assessment through time-consuming and costly experimental verification processes.

The methods and systems described herein can be used to define gene cluster boundaries, i.e., to identify functionally related genes that are co-localized on a chromosome. The gene clusters, or protein products thereof, may participate in a variety of cellular functions, such as biosynthesis (e.g., secondary and primary metabolism), immunity, cell structure, scavenging, energy and sensing. In particular, the methods described herein can be used to define borders of a gene cluster (e.g., a BGC) in different genomes by identifying genes that are associated with the gene cluster.

Furthermore, the methods and systems can be used to identify a resistance gene (e.g., a gene that confers resistance to the action of the secondary metabolite produced by a BGC to the host organism) which is embedded in the BGC required for the production of the secondary metabolite. The identification of BGC embedded resistance genes enables the de-orphanization of the BGC-encoded small molecule's protein target (ETaG product), which may have a homolog in a mammalian genome. The mammalian homologs of the ETaG may serve as candidate therapeutic targets, and the secondary metabolite can provide small molecule scaffolds for developing modulators against such mammalian homologs.

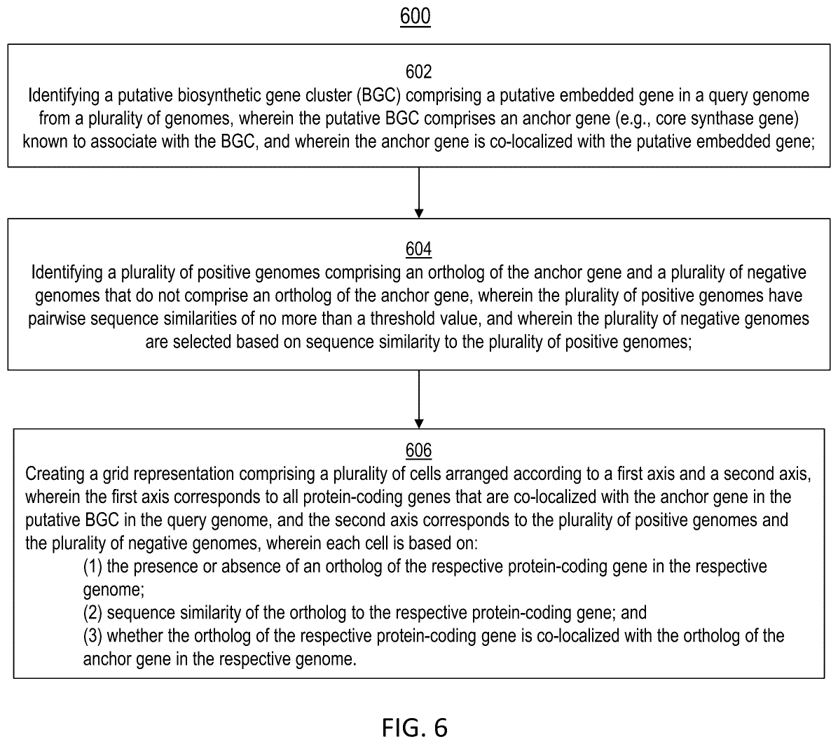

FIG. 6 illustrates an exemplary method 600 for generating a grid representation (e.g., a heat map) of genomic data that can be input into a machine-learning algorithm (such as an artificial neural network (ANN), a convolutional neural network (CNN), a multilayer perceptron (MLP), a deep neural network (DNN), an LSTM, a vision transformer model, a generative adversarial network (GAN) model, a variational autoencoder model, a latent diffusion model. etc.) to determine a likelihood that a putative embedded gene (e.g., a pETaG) is associated with a gene cluster (e.g., BGC) in a genome.

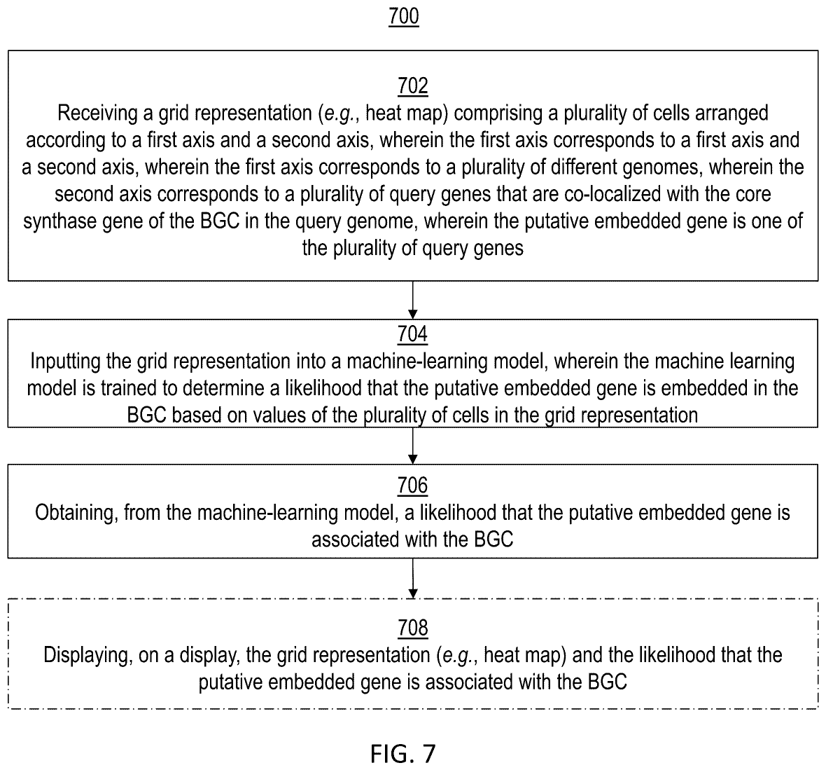

FIG. 7 illustrates an exemplary method 700 for determining a likelihood that a putative embedded gene is associated with a gene cluster (e.g., a BGC). Process 600 and process 700 are performed, for example, using one or more electronic devices implementing a software platform. In some examples, process 600 and/or process 700 is performed using a client-server system, and the blocks of process 600 and/or process 700 are divided up in any manner between the server and one or more client devices. In some examples, process 600 and/or process 700 is performed using only a client device or only multiple client devices. In process 600 and/or 700, some blocks are, optionally, combined, the order of some blocks is, optionally, changed, and some blocks are, optionally, omitted. In some examples, additional steps may be performed in combination with the process 600 and/or process 700. Accordingly, the operations as illustrated (and described in greater detail below) are exemplary by nature and, as such, should not be viewed as limiting.

### Grid Representation

At block 702 of FIG. 7, an exemplary system (e.g., comprising one or more electronic devices) receives a grid representation of genomic data (such as a heat map representation) comprising a plurality of cells arranged according to a first axis and a second axis, wherein the first axis corresponds to a plurality of different genomes (e.g., non-mammalian genomes), wherein the second axis corresponds to a plurality of query genes that are co-localized with the anchor gene (e.g., a core synthase gene) of a gene cluster (e.g., a BGC) in the query (or reference) genome, wherein the putative embedded gene is one of the plurality of query genes. Each cell in the grid representation has a value based on the following: (i) whether an ortholog of the respective query gene (i.e., the query gene corresponding to the cell) is present or absent in the respective genome (i.e., the genome corresponding to the cell); (ii) sequence similarity of the ortholog to the respective query gene; and (iii) whether the ortholog of the respective query gene is co-localized with the ortholog of the anchor gene (e.g., core synthase gene) in the respective genome.

The grid representation described herein can take any of a variety of forms known to those of skill in the art. For example, the grid representation may be a data matrix, such as a two-dimensional data matrix (e.g., a table or array) arranged according to the first axis and the second axis and having the values described herein for each of the cells in the data matrix. In some instances, the grid representation comprises one or more matrices (e.g., tables) of data. For example, each set of values (i.e., (i) whether an ortholog of the respective query gene (i.e., the query gene corresponding to the cell) is present or absent in the respective genome (i.e., the genome corresponding to the cell); (ii) sequence similarity of the ortholog to the respective query gene; and (iii) whether the ortholog of the respective query gene is co-localized with the ortholog of the anchor gene (e.g., core synthase gene) in the respective genome) of the cells in the grid representation, or combinations thereof, may be stored in a separate table and used as an input for the machine learning-based methods described herein. In some instances, the grid representation may be a physical representation of the underlying data matrix, for example, a heat map, which facilitates visualization of the data.

With respect to each query gene in the query (or reference) genome, an ortholog in any given genome (e.g., a target genome) may be identified based on the coding sequence of the query gene or the protein sequence encoded by the query gene, or based on phylogenetic relationships using known methods in the art. For example, an ortholog of a query gene in a given genome may be a gene in the given genome that encodes a protein having the highest sequence similarity to the query gene, or for which the sequence similarity is above a pre-determined threshold. Sequence similarities can be quantified by any of a variety of parameters known to those of skill in the art, including percent sequence identity, percent sequence homology, bitscore, and e-value. The pre-determined threshold may be a percent sequence identity or percent sequence homology of, for example, at least about any one of 20%, 30%, 40%, 50%, 60%, 70%, 80%, 85%, 90%, 95%, 96%, 97%, 98%, 99% or higher.

In some instances, an ortholog of a query gene in a given genome may be a bidirectional best hit (BBH) of the query gene in the given genome. The method for identifying a BBH has been described, for example, in Moreno-Hagelsieb G, Latimer, K., Bioinformatics. 2008 Feb. 1; 24(3):319-24. For example, to identify a BBH of a query gene in a given genome, the given genome is first searched for the gene (“putative BBH”) encoding a protein having the highest sequence similarity to the protein encoded by the query gene. This search is followed by a reciprocal search, in which the query genome is searched for the gene encoding a protein having the highest sequence similarity to the putative BBH identified in the query genome. If the gene identified in the reciprocal search is the original query gene, the putative BBH is a true BBH. Alternatively, an ortholog of a query gene in a given genome may be identified using a reciprocal smallest distance method, e.g., as described in Wall D P, Deluca T, Methods Mol Biol. 2007; 396: 95-110.

The query genes, including the putative embedded genes, in a query (or reference) genome are co-localized with an anchor gene (e.g., core synthase gene) of a gene cluster, e.g., a BGC. Two genes may be considered co-localized if one gene is within a specified distance, or proximity zone, of the other. In some instances, the distance between two genes can be considered, for example, the shortest distance between the genomic coordinates of the two genes. For example, if gene A resides on the +strand and comprises a sequence ranging from positions 1 to 100, and gene B resides on the − strand and comprises a sequence ranging from positions 300 to 200 (i.e., where position 300 is the start of gene sequence B due to its position on the −strand), then the distance between the two genes is 200-100=100 bp. In some instances, the distance between two genes can be considered the longest distance between the genomic coordinates of the two genes. In some instances, the distance between the two genes can be considered the distance between the midpoint genomic coordinates for the two genes. A putative embedded gene in a target genome is co-localized with an anchor gene (e.g., core synthase gene) of a BGC in the target genome when the putative embedded gene is within a specified proximity zone relative to the anchor gene (e.g., core synthase gene) in the BGC of the target genome. In some instances, a proximity zone is about 1-100 kb, for example, no more than about 1, 2, 3, 4, 5, 6, 7, 8, 9, 10, 15, 20, 30, 40, 50, 60, 70, 80, 90, or 100 kb upstream or downstream of an anchor gene (e.g., core synthase gene) in a BGC. In some instances, a proximity zone is about 1-10 kb, for example, no more than 1, 2, 3, 4, 5, 6, 7, 8, 9, or 10 kb upstream or downstream of an anchor gene (e.g., core synthase gene) in a gene cluster (e.g., a BGC). In some instances, a proximity zone is no more than 5 kb upstream or downstream of a gene. In some instances, a proximity zone is no more than 10 kb upstream or downstream of a gene. In some instances, a proximity zone is no more than 15 kb upstream or downstream of a gene. In some instances, a proximity zone is no more than 20 kb upstream or downstream of a gene. In some instances, a proximity zone is no more than 25 kb upstream or downstream of a gene. In some instances, a proximity zone is no more than 30 kb upstream or downstream of a gene. In some instances, a proximity zone is no more than 35 kb upstream or downstream of a gene. In some instances, a proximity zone is no more than 40 kb upstream or downstream of a gene. In some instances, a proximity zone is no more than 45 kb upstream or downstream of a gene. In some instances, a proximity zone is no more than 50 kb upstream or downstream of a gene.

A putative BGC, for example, includes both biosynthetic genes and non-biosynthetic genes, and may further include pseudogenes (e.g., nonfunctional segments of DNA that resemble functional genes). Curated libraries of profile Hidden Markov Model (pHMMs) of biosynthetic domains of BGCs (i.e., probabilistic models that capture the biological diversity of the biosynthetic domains based on a position-dependent scoring of multiple sequence alignments for the corresponding protein or nucleic acid sequences) are known in the art and can be used to identify biosynthetic genes that are clustered in a genome. An anchor gene may be any one of the signature genes associated with a BGC. For example, an anchor gene may be the core synthase gene in a BGC, which is the largest biosynthetic gene in a BGC, or the anchor gene may be the core synthase gene closest to a query gene. Alternatively, an anchor gene may be a non-biosynthetic gene that is known to associate with a BGC, for example, a gene encoding a transporter for the secondary metabolite produced by a BGC. In some embodiments, the plurality of genes in a putative BGC may be identified by extending a window of predetermined length upstream and downstream from an anchor gene (e.g., core synthase gene). In some embodiments, the plurality of genes in a putative BGC of a given genome can be identified using bioinformatics methods, such as antiSMASH.

For example, a grid representation may have a first axis (e.g., Y-axis) corresponding to genomes Q₁ to Qₙ and a second axis (e.g., X-axis) corresponding to genes G₁ to Gₘ. A cell corresponding to genome Qᵢ(1≤i≤n) and gene Gj (1≤i≤n) has a value and a color selected from a first color, a second color and a third color according to the following:
(i) if Qᵢ does not have a BBH in Gⱼ, the color is the first color and the value is 0; or
(ii) if Qᵢ has a BBH in Gⱼ, the value is based on percentage sequence identity of Gⱼ to the BBH of Gⱼ in Qᵢ, and
   (ii-1) if the BBH in Gⱼ is co-localized with the BBH of the core synthase gene in Qᵢ, the color is the second color, or
   (ii-2) if the BBH in Gⱼ is not co-localized with the BBH of the core synthase gene in Qᵢ, the color is the third color.

The grid representation may be hierarchically clustered in order to assist visualization and manual annotation. For example, the grid representation be clustered based on the pairwise sequence identity or homology among the genomes, phylogeny of the genomes, or the presence or absence of orthologs corresponding to all query genes in the grid representation. In some instances, the first axis of the grid representation (e.g., heat map) is organized according to a phylogenetic tree of the plurality of genomes represented in the grid representation.

In some instances, a putative embedded gene is a putative embedded target gene (pETaG), which encodes a homolog of a mammalian protein of interest, such as a homolog of a human protein of interest. In some instances, a pETaG is homologous to an expressed mammalian nucleic acid sequence. In some instances, a mammalian nucleic acid sequence is an expressed mammalian nucleic sequence. In some instances, a mammalian nucleic acid sequence is a mammalian gene. In some instances, a mammalian nucleic acid sequence is an expressed mammalian gene. In some instances, a mammalian nucleic acid is a human nucleic acid sequence. In some instances, a human nucleic acid sequence is an expressed human nucleic acid sequence. In some instances, a human nucleic acid sequence is a human gene. In some instances, a human nucleic acid sequence is an expressed human gene.

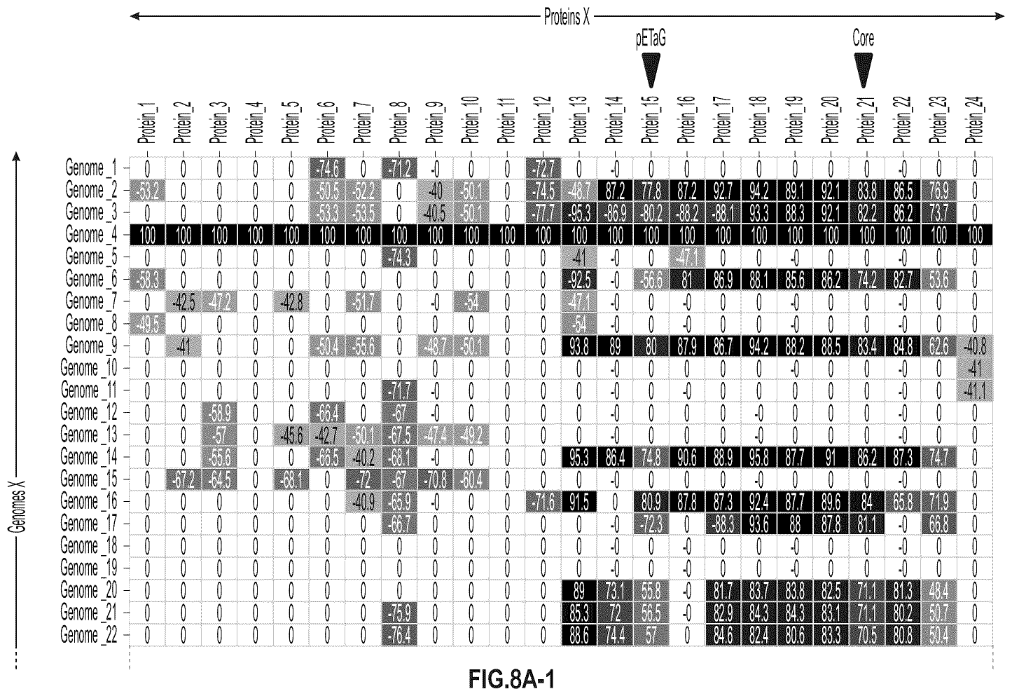

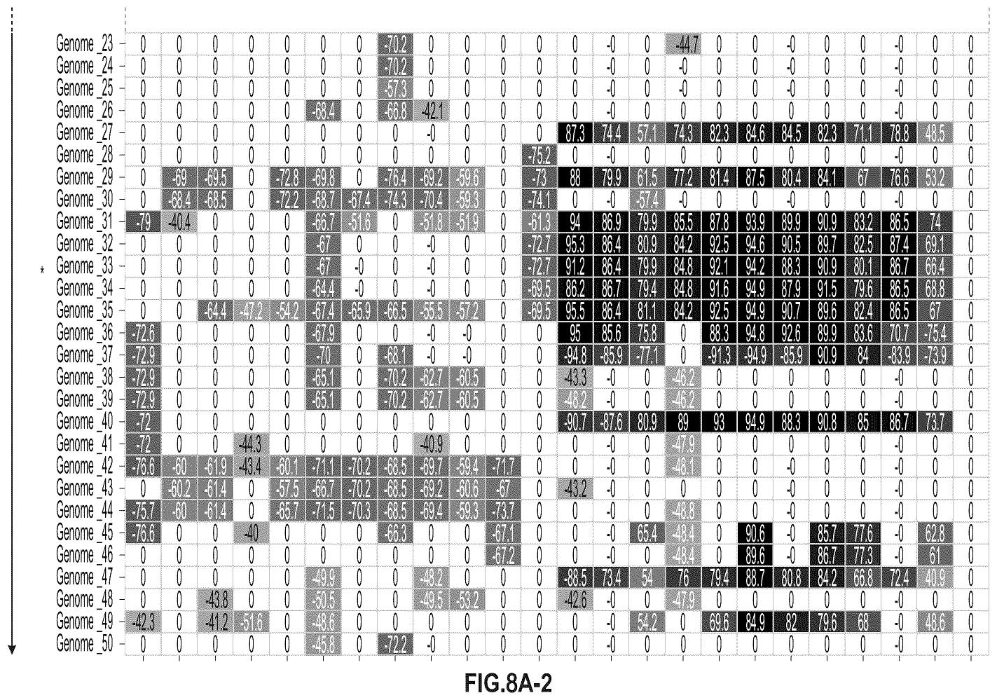

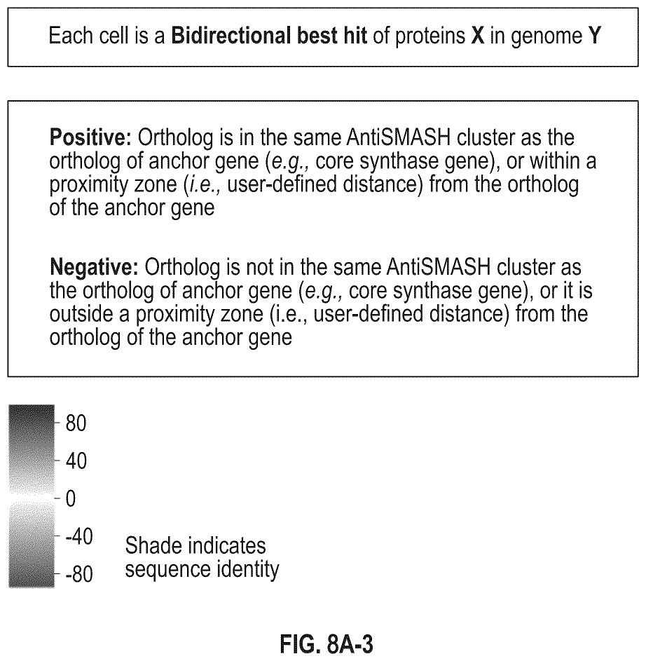

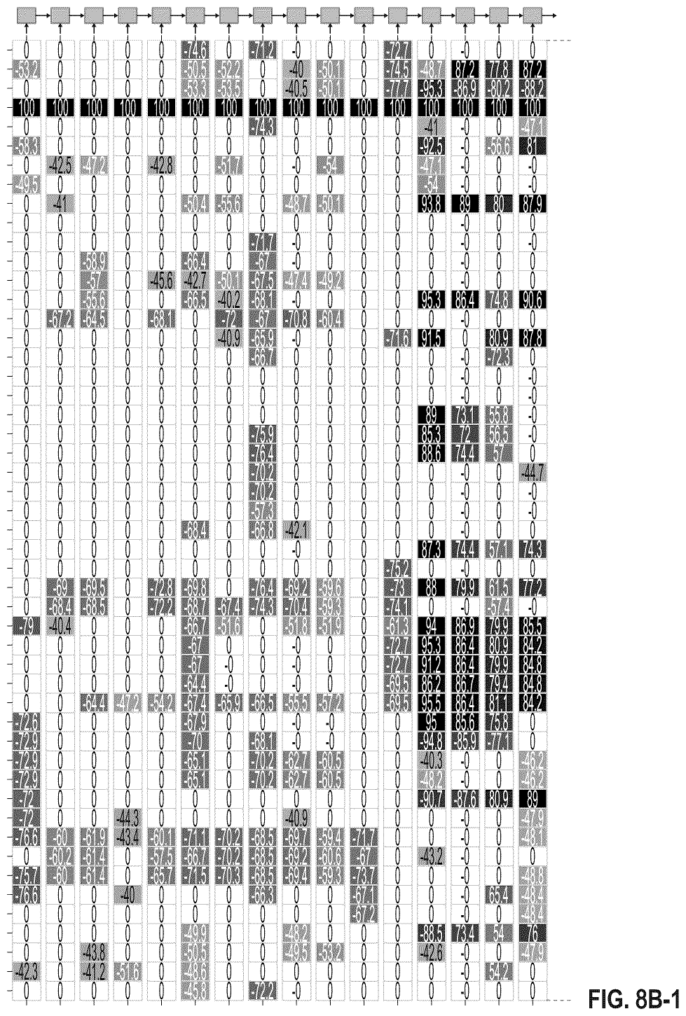

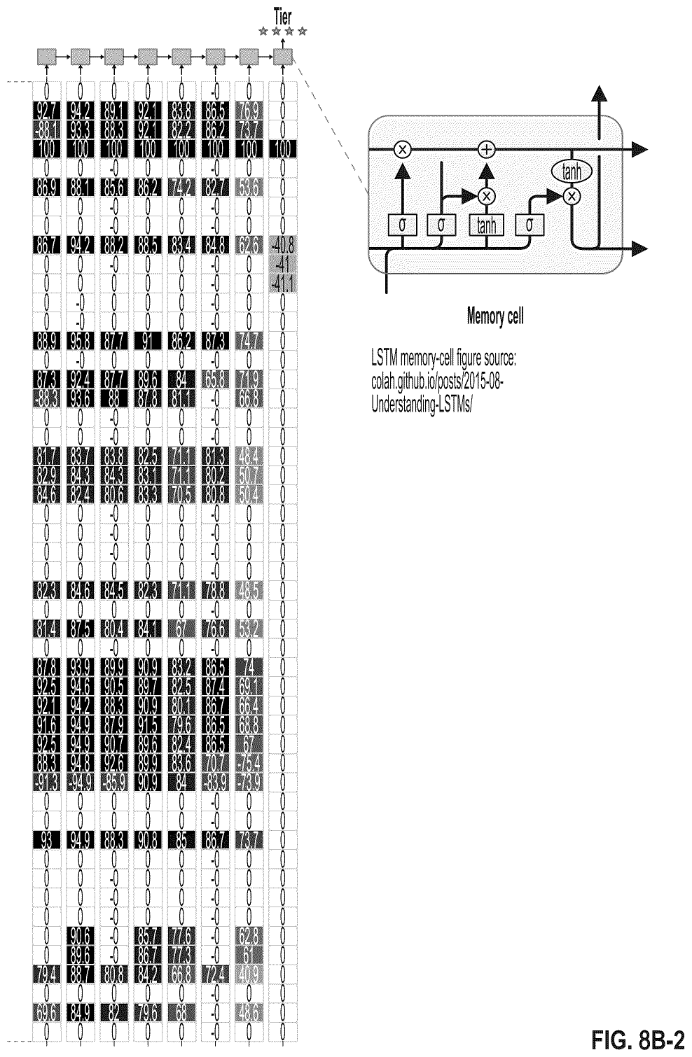

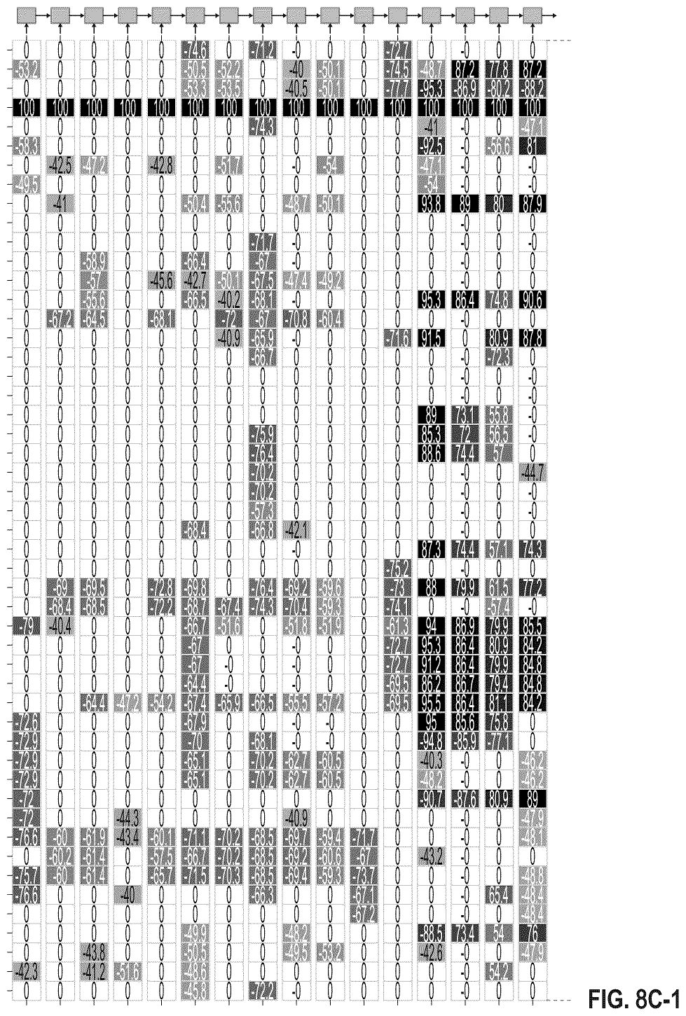

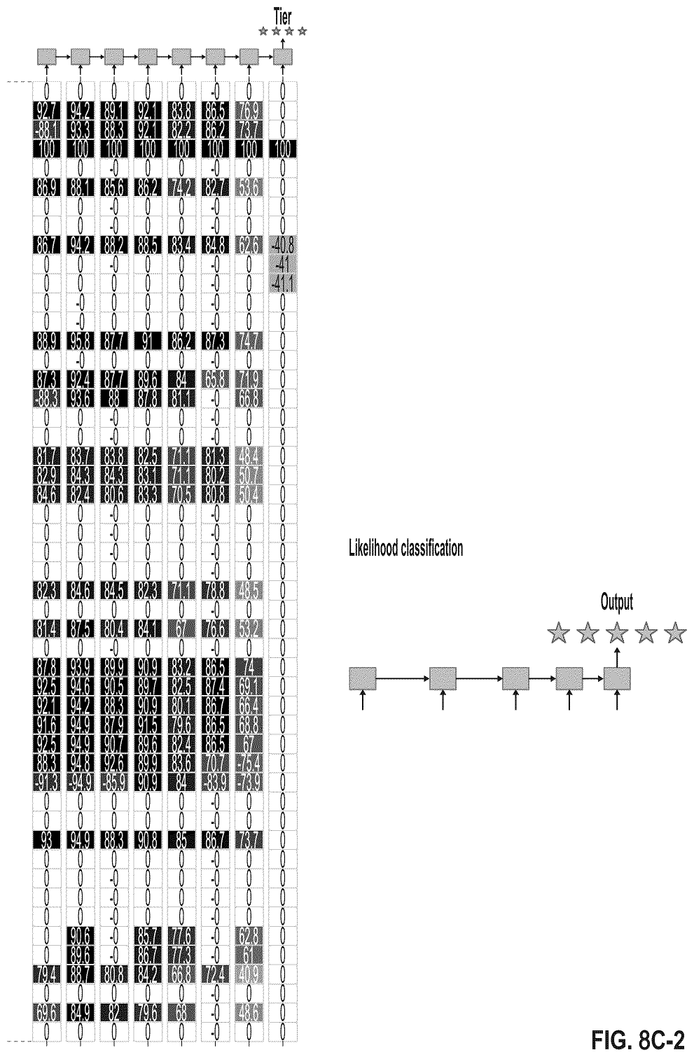

An example of a genomic heat map is shown in FIGS. 8A-1 to 8A-3. This example illustrates a heatmap for a pETaG in a BGC identified by antiSMASH in the genome marked by an asterisk (*). Each of the columns along the X-axis represents a protein (“Protein X”) encoded by a query gene in the BGC identified in the query (or reference) genome marked by an asterisk (*). In some instances, the BGC is identified by antiSMASH. In some instances, the genes in the BGC are within a proximity zone of 20 kb from the core synthase gene of the BGC (i.e., within ±20 kb of the core synthase gene). The columns corresponding to a pETaG and the core synthase gene are marked with arrows. Each of the rows along the Y-axis represents a unique genome (“Genome Y”) selected from the genome database. Half of the genomes contain a BBH of the core synthase gene, and are designated positive genomes for the purpose of identifying genes in a putative BGC. Half of the genomes do not contain a BBH of the core synthase gene, and are designated as negative genomes for the purpose of identifying the genes in a putative BGC. Each cell is colored or shaded according to the presence or absence of a BBH of the respective query gene, and the percentage sequence identity (numbers in cells) of the BBH to the respective query gene. For example, cell (X,Y) is blank if there is no BBH of Protein X in Genome Y; cell (X,Y) is, e.g., blue or positive, if there is a BBH of Protein X in Genome Y and the BBH is in the same antiSMASH BGC cluster as the BBH of the core synthase gene in Genome Y; or cell (X, Y) is e.g., red or negative, if there is a BBH of Protein X in Genome Y and the BBH is not in the same antiSMASH BGC cluster as the BBH of the core synthase gene in Genome Y. The intensity of the red or blue color (or grayscale shading) of cell (X, Y) is based on percentage sequence identity of the BBH of Protein X in Genome Y to Protein X. The heat map is hierarchically clustered based on pairwise sequence identities among the genomes.

The genomes shown in the grid representation may each correspond to an assembled genome, or a plurality of genome fragments obtained from genome sequencing. In some instances, the genomes are annotated using bioinformatics tools, such as antiSMASH, prior to the analysis using any one of the methods described herein. For example, a database of genomes can be constructed so that all putative biosynthetic gene clusters have been identified and annotated. For example, genome fragments containing the putative BGCs, instead of full genomes, may be queried in one or more steps of the methods described herein. For example, co-localization of genes (e.g., orthologs of query genes) with an anchor gene (e.g., core synthase gene) can be determined based on the putative BGC annotations in the genomes.

The methods described herein are suitable for any genomes containing gene clusters, e.g., BGCs. Bacterial genomes, plant genomes, and fungal genomes are known to encode biosynthetic gene clusters. In some embodiments, the query genome and the plurality of queried genomes used to generate the grid representation belong to the same kingdom. In some embodiments, the query (or reference) genome and the plurality of queried (or target) genomes used to generate the grid representation belong to different kingdoms. Suitable genomes include, but are not limited to genomes of Archaea, Protozoa, Chromista (e.g., brown algae, diatoms, crypophytes, etc.), Plantae (e.g., green algae and plants), Fungi, and Animalia. In some embodiments, the query genome and the plurality of queried genomes are fungal genomes, such as genomes of different fungal strains. In some embodiments, the query genome and the plurality of queried genomes are bacterial genomes, such as genomes of different bacterial strains. In some embodiments, the query genome and the plurality of queried genomes are plant genomes, such as genomes of different plant strains. Without wishing to be bound by any theory or hypothesis, fungal genomes are eukaryotic genomes that are phylogenetically more related to mammalian genomes than bacterial or plant genomes. Thus, fungal genomes may be preferred for identification of ETaGs that correspond to human target genes for the secondary metabolites produced by the BGCs harboring the ETaGs.

At least 2 genomes are required for building the grid representation. In some instances, the first axis of the grid representation corresponds to at least about any one of 10, 15, 20, 25, 30, 35, 40, 45, 50, 60, 70, 80, 90, 100, 150, 200, 250 or more genomes. In some instances, the first axis of the grid representation corresponds to at least 20 genomes. In some instances, the first axis of the grid representation corresponds to about 50 genomes. Although a large number of genomes may provide more comparative genomic information, it also requires a large amount of computational power and time. Thus, it may be desirable to sample a representative set of genomes that are diverse with respect to each other in terms of their sequence similarities and/or phylogenetic relationships to generate the grid representation to strike a balance between performance of the methods (e.g., accuracy of prediction) and computational resource.

The genomes shown in the grid representation may include “positive genomes” and “negative genomes.” Positive genomes are those genomes having orthologs of the anchor gene, such as the core synthase gene, in the query (or reference) genome. Negative genomes are those genomes that do not have orthologs of the anchor gene, such as the core synthase gene, in the query (or reference) genome. In some instances, the positive and negative genomes are selected from a database of genomes. In some instances, the plurality of genomes used to build the grid representation comprises a plurality of positive genomes each having an ortholog (e.g., BBH) of the anchor gene (e.g., core synthase gene), and a plurality of negative genomes that do not have an ortholog (e.g., BBH) of the anchor gene (e.g., core synthase gene). In some instances, the positive genomes are selected from a genome database by identifying genomes having an ortholog (e.g., BBH) of the anchor gene (e.g., core synthase gene), and the negative genomes are selected form genome database by identifying genomes without an ortholog (e.g., BBH) for the anchor gene (e.g., core synthase gene). Negative genomes may be phylogenetically adjacent to the selected positive genomes. In some embodiments, the number of positive genomes and the number of negative genomes are equal to each other.

The positive and negative genomes may be selected from a database having a large number of genomes. For example, a database may contain at least 2, 10, 100, 500, 1000, 5000, 10000, 15000, 20000, 25000, 30000, 35000, 40000, 45000, 50000, 100000, 200000, 500000, 1000000, or more than 1000000 genomes. Selection of the positive and negative genomes from a large genome database may require clustering of the genomes in order to allow sampling of diverse genomes, including positive and negative genomes, from the database. For example, in some instances the database genomes may be clustered according to the sequence similarities of one or more single copy genes in the genomes, or the sequence similarities of orthologs of the anchor gene (e.g., core synthase gene), or the putative embedded gene (e.g., a pETaG) in the genomes. The clustering may be performed using an unsupervised clustering method. The unsupervised clustering method may comprise the use of, for example, a Markov Cluster algorithm (MCL), a Restricted Neighborhood Search Cluster (RNSC) algorithm, an Affinity Propagation clustering algorithm, a Spectral clustering algorithm, a k-means clustering algorithm, or any other method known in the art. The clustering may alternatively comprise the use of a supervised clustering method that is known in the art, such as supervised k-means clustering or semi-supervised spectral clustering. The threshold for clustering may be determined by a predetermined goal for the number of clusters. For example, the threshold for clustering may be a pre-determined sequence similarity level among the group of genomes, e.g., requiring sequence similarities between different groups of genomes to be no more than about any one of 99.5%, 99%, 98%, 95%, 90%, 85%, 80%, 75%, 70%, 65%, 60%, 50%, 40%, 30%, or less. In some embodiments, it may be desirable to select positive genomes in which the pairwise percent sequence similarities (e.g., sequence identity) of orthologs of one or more single copy genes in the positive genomes to be more than about any one of 99.5%, 99%, 98%, 97%, 96%, 95%, 94%, 93%, 92%, 91%, 90%, 85%, 80%, 70%, 60%, 50%, 40%, 30%, or less. In some embodiments, it may be desirable to select negative genomes in which the pairwise percent sequence similarities (e.g., sequence identity) of orthologs of one or more single copy genes in the negative genomes to be more than about any one of 99.5%, 99%, 98%, 97%, 96%, 95%, 94%, 93%, 92%, 91%, 90%, 85%, 80%, 70%, 60%, 50%, 40%, 30%, or less. A representative genome from each of the clusters can be further selected for use in the analytical steps as described herein. In some embodiments, a negative genome is selected from a database by identifying the genome having the highest sequence similarity to a positive genome, but that lacks an ortholog of the anchor gene (e.g., core synthase gene).

For example, a grid representation may be built based on a number, n, of positive genomes selected from a database of m genomes. As a first step, a number of positive genomes, m₁, having an ortholog (e.g., BBH) of the anchor gene (e.g., core synthase gene) and a number, (m−m₁), of negative genomes without an ortholog (e.g., BBH) of the anchor gene (e.g., core synthase gene) are identified from the m number of genomes in the database. In the situation where mₗ is larger than n, the mₗ positive genomes are clustered using MCL based on the average sequence similarities of one or more single copy genes (e.g., identified using the BUSCO tool, see, busco.ezlab.org) among the positive genomes into n clusters. One positive genome is then selected from each of the n clusters to provide n positive genomes for building the grid representation. Each of the n negative genomes is selected by identifying the most similar genome (e.g., the genome with the highest sequence similarity to, or with the shortest phylogenetic distance from) a selected positive genome among the (m−m₁) negative genomes. This genome selection method results in a grid representation built from n positive genomes and n negative genomes.

In the situation where m₁ is smaller than n, then more negative genomes may be selected than positive genomes in order to build a grid representation having a total of 2n genomes. In this case, the (m−m₁) negative genomes may be clustered into (2n−m₁) clusters, and one negative genome is chosen from each cluster. Alternatively, for each of the mₗ positive genomes, more than one negative genome that is closely related to the positive genome is chosen based on their sequence similarities or phylogenetic distances to the positive genome, so that a total of 2n−m₁ negative genomes are selected.

The grid representation may be generated, for example, using the method illustrated in FIG. 6.

As a first optional step, resource files can be prepared for the downstream computational processes. Exemplary resource files include a pairwise genome comparison file, a file (e.g., FASTA file) containing relevant proteins or genes from target genomes (i.e., genomes selected for the analysis), and optionally, a resource file containing Clusters of Orthologous Groups of proteins or genes (COGs).

A pairwise genome comparison file is a file created to show the homology relationship between all pairs of genomes in a database. Genome similarities can be determined based on either pairwise genome sequence similarities, or pairwise phylogenetic distances between genomes.

In some examples, pairwise identities or similarities among genomes can be determined by either comparing whole genome sequences, or by comparing sequences of subsets of proteins or genes. For example, to determine whole genome sequence identities, the entire genomes can be aligned and the pairwise identities between the alignments are computed. Alternatively, pairwise genome identities can be calculated by comparing single copy proteins (i.e., amino acid sequences) or genes (i.e., nucleotide sequences) shared between the pair of genomes. In some preferred embodiments, single copy proteins or genes are used as duplicated or fragmented proteins may provide misleading estimates of genome homology. Single copy proteins or genes in a genome can be annotated using BUSCO (doi.org/10.1093/molbev/msab199), or specific known single-copy proteins or genes can be used to determine genome sequence similarities. In some instances, single-copy proteins can be identified using known bioinformatic tools such as OrthoMCL, OrthoFinder, or PanX. A subset or all single copy proteins or genes shared between the genomes are individually aligned, trimmed, and concatenated to create a super-alignment. The pairwise identity is the number of identical residues in the super-alignment. Alternatively, a similarity score can be calculated based on substitution matrices, such as BLOSUM and PAM if protein sequences are used to determine sequence similarities.

In other examples, genome similarities are determined based on phylogenetic distances between the genomes. To determine phylogenetic distances, a set of single-copy proteins (i.e., amino acid sequences) or genes (i.e., nucleotide sequences) can be individually aligned using any alignment software such as MAFFT, MUSCLE, or ClustalW, trimmed using any sequence trimming software such as trimAI, GBlocks, or ClipKIT, and concatenated to create a super-alignment. The super-alignment can be used by any phylogenetic tree building software such as FastTree, IQ-TREE, RAxML, MEGA, MrBayes, BEAST, or PAUP to provide a phylogenetic tree of the genomes. Trees can be constructed using different algorithms, such as maximum likelihood algorithms, parsimony algorithms, neighbor joining algorithms, distance matrix algorithms, or Bayesian inference algorithms. Alternatively, a gene-coalescent phylogenetic model approach can be used to re-construct the phylogeny instead of the super-alignment approach.

In some examples, a FASTA file containing all protein or gene sequences from target genomes is created as an input resource file. Alternatively, a smaller subset of proteins, such as proteins in putative gene clusters (e.g., a putative BGCs) based on bioinformatics predictions, can be supplied as an input instead of all proteins from all genomes. The putative BGCs, for example, can be predicted using publicly available BGC prediction tools such as antiSMASH, SMURF, TOUCAN, deepBGC, or using custom BGC prediction tools. The FASTA file may contain either protein sequences or nucleic acid sequences as sequence similarities (e.g., homology) may be determined using either protein sequences or nucleic acid sequences.

Clusters of Orthologous Groups (COG) of gene cluster (e.g., BGC) proteins or genes may be provided as an optional resource file. Members of the same COG are presumed to have orthologous functions. COGs can be created using protein clustering tools such as USEARCH, CD-HIT, and MMseqs. Alternatively, COGs can be generated using clustering algorithms such as MCL or SiLiX after performing sequence (e.g., amino acid or nucleotide) alignment searches with BLAST, ggsearch, or Diamond. COGS can also be identified using custom developed scripts or using known bioinformatics tools such as OrthoMCL, OrthoFinder, or PanX. COGs can also be defined as gene families or protein families. The COG file may, for example, contain protein or gene IDs from the same COG in each row of a table.

At block 602 of FIG. 6, the method for generating a grid representation comprises identifying a putative gene cluster (e.g., a putative biosynthetic gene cluster (BGC)) comprising a putative embedded gene in a query (or reference) genome from a plurality of genomes, wherein the putative gene cluster comprises an anchor gene (e.g., core synthase gene) known to associate with the gene cluster (e.g., the BGC), and wherein the anchor gene is co-localized with the putative embedded gene. In some examples, the method comprises identifying the longest biosynthetic or structural gene in the putative gene cluster as the anchor gene. The resource files can be used to perform the step of block 602. This step establishes the first axis of a grid representation, e.g., the X-axis of a heat map, which corresponds to a plurality of query genes that are co-localized with the putative gene cluster (e.g., BGC) in the query genome and that comprises the putative embedded gene. Co-localization may be determined either based on putative BGC annotations, or based on the distance between two genes, e.g., two genes within a specified proximity zone of no more than about 50 kb, or no more than about 20 kb.

For example, the X-axis of a heat map can be established based on a single protein or gene ID of interest (e.g., a gene ID corresponding to a pETaG), or based on multiple protein or gene IDs of interest as input. If multiple protein or gene IDs are input, then correlation and co-localization of these genes (along with flanking genes) with respect to each other across multiple genomes are determined. For example, the multiple protein or gene IDs of interest may correspond with an ETaG and a core synthase gene in a query (or reference) genome. If a single protein or gene ID is used as an input, then one can determine if the flanking genes surrounding it are co-localized across multiple genomes. The X-axis corresponds to the search region that contains the flanking genes of the protein or gene(s) (e.g., pETaG) of interest. The search region can be defined through either a predicted BGC or based on a coordinate location on the genomes. Putative BGCs can be predicted using tools such as antiSMASH, SMURF (dx.doi.org/10.1016/j.fgb.2010.06.003), TOUCAN (doi.org/10.1093/nargab/lgaa098), deepBGC (doi.org/10.1093/nar/gkz654), or other custom search algorithms.

If a predefined region is used as an input, then it is assumed that the specified input protein(s) or corresponding gene ID(s) reside within the region. Alternatively, a custom flanking distance (i.e., a proximity zone distance) specified in units of, e.g., base pairs (bp) can be used to identify a genome window region that comprises a certain number of bps upstream and downstream flanking either side of the specified protein or gene ID(s). All proteins (or genes) that are located within the defined region are assigned as X-axis proteins (or genes). The proteins (or genes) within the region are labeled. If the input protein ID is a single protein, the input label is used as the label for the protein ID. For example, one can input a core synthase gene and determine the genes that are co-localized with the core synthase. If the input protein ID is a single protein and it is desired to determine its correlation with another gene (for which the ID was not input), then another specified input can be used to identify genes within the region to label as such. For example, if a gene of interest corresponding to an ETaG is used as an input and it is desired to determine if it is correlated with a core synthase, one can identify a search for core synthases and label identified proteins as such. Proteins can be searched based on gene annotations. If there are multiple target proteins within the region that match the requested search criteria, several options can be utilized. For example, heatmaps for each of the target proteins can be created. A target protein can be selected based on length or proximity of the target protein to the input protein ID. If multiple protein or gene IDs are used as an input, then the protein IDs are labeled based on their input labels. For example, one input protein can be labeled as the ETaG and the other input protein labeled as a core synthase.

At block 604 of FIG. 6, the method comprises obtaining a plurality of positive genomes comprising an ortholog of the core synthase gene and a plurality of negative genomes that do not comprise an ortholog of the core synthase gene, wherein the plurality of negative genomes are selected based on sequence similarities or phylogenetic distances to the plurality of positive genomes. The resource files can be used to perform the step of block 404. This step establishes the second axis of the grid representation, e.g., Y-axis of a heat map, which corresponds to the target genomes, including a plurality of positive genomes and a plurality of negative genomes.

For example, to establish the Y-axis of a heat map, positive and negative genome IDs can be obtained as follows. To establish positive genome IDs, protein homologs of the core synthase ID are searched for by running protein sequence alignment tools such as ggsearch, BLASTp, or Diamond-blastp against a set of genomes. Alternatively, genes (i.e., nucleotide sequences) can be used to find homologs for the gene of the core synthase in the set of genomes using tools such as ggsearch or BLASTn. Protein IDs falling within a specified range of minimum and maximum sequence identity are identified. A specified number of protein homologs from those having the highest sequence identity to the query core synthase may be selected. Core synthase homologs can be de-replicated by selecting representatives from protein clusters created by using protein clustering tools with a specified cutoff. Alternatively, dereplication can also be done using a BUSCO pairwise cutoff, phylogenetic distance cutoff, or taxonomic classifications. If a phylogenetic tree is used as a genome homology resource file, it can be traversed to select a diverse set of positive genomes that pass the criteria for presence of protein homologs. Alternatively, positive genomes can be selected based on pairwise BUSCO identity cutoffs, phylogenetic distance or clades, or taxonomic classifications (e.g., one isolate per species or one species per genera or family from genomes with protein homologs). These methods can be utilized to help ensure selection of a diverse set of genomes, which provides greater accuracy in identifying co-localized genes and avoids confounding results with multiple genomes of the same species, which can bias the results towards co-localization and increase the false positive rate. Each genome ID from the selected core synthase homologs is obtained and assigned as a positive genome.

To obtain negative genome IDs, the genome ID with the highest sequence identity or the closest phylogenetic distance to each positive genome may be selected using the pairwise genome homology file as an input. If a genome contains core synthase homologs within a specified range of sequence identity, the genome is dropped from the list of candidates and the search skips to the next candidate genome. If a phylogenetic tree is used as the homology resource file, then the tree can be traversed to find the closest negative genome to each positive genome. The positive and negative genome IDs are combined and assigned as the Y-axis genome IDs.

Optionally, files containing all protein (or nucleotide) sequences and gene annotation files (GFF, GTF, GenBank, or similar) related to Y-axis genomes are obtained. If gene cluster (e.g., BGC) predictions are used to define the search region, then gene cluster (e.g., BGC) information for the Y-axis genomes including gene cluster (e.g., BGC) IDs, cluster numbers, and protein (or gene) IDs located in the gene cluster (e.g., BGC) is stored as a resource file.

In some examples, a linkage matrix (also known as a cladogram) is built to visually show distances between all genomes of the Y-axis. The linkage matrix can be created using the pairwise homology matrix of the Y-axis genomes from the genome homology resource file (e.g., pairwise identities, similarities, or phylogenetic distance). A hierarchical clustering method can be used with the pairwise homology matrix to create a linkage matrix. Alternatively, the presence/absence of BBHs or forward alignment results of the X-axis protein homologs can be used with a hierarchical clustering method to create a linkage matrix. Alternatively, a phylogenetic tree can be used. The phylogenetic tree can be created from either a set of proteins (i.e., amino acid sequences) or transcripts (i.e., nucleotide sequences).

At block 606 of FIG. 6, the method comprises creating a grid representation comprising a plurality of cells arranged according to a first axis and a second axis, wherein the first axis corresponds to all protein-coding genes (e.g., query genes) that are co-localized with the anchor gene in the putative gene cluster (e.g., a putative BGC) in the query (or reference) genome, and the second axis corresponds to the plurality of positive genomes and the plurality of negative genomes, wherein each cell is based on: (1) the presence or absence of an ortholog of the respective protein-coding gene in the respective genome; (2) sequence similarity of the ortholog to the respective protein-coding gene; and (3) whether the ortholog of the respective protein-coding gene is co-localized with the ortholog of the anchor gene in the respective genome.

For example, the following steps can be used to create a heat map matrix. First, bi-directional best hits (BBH) results for X-axis proteins (or genes) in all Y-axis genomes are obtained to provide a BBH table. BBH is identified as a pair of proteins (or genes) from two different genomes that are more similar to each other than either is to any other gene in the other genome. BBH can be useful in identifying the true ortholog of a given protein (or gene), which is particularly helpful in identifying the current ortholog of genes that have had duplication events (such as ETaGs). Identification of BBHs involves a forward alignment step and a backward alignment step. In the forward alignment step, a sequence alignment tool is run using each X-axis protein as a query against the protein FASTA file of each Y-axis genome with specified cutoffs. Sequence alignment tools such as ggsearch, BLASTp, or Diamond-blastp can be used.

Alternatively, genes or transcripts (i.e., nucleotide sequences) can be used instead of protein sequences for the forward alignment step using tools such as ggsearch or BLASTn. The protein (or gene) ID of the best match from each alignment is stored in a table with X-axis proteins (or genes) as columns and Y-axis genomes as indices. The sequence identity of the best match from each alignment is stored in a table with X-axis proteins as columns and Y-axis genomes as indices. In the backward alignment step, a backward alignment is run using protein sequence alignment tools such as ggsearch, BLASTp, or Diamond-blastp. Each protein stored as the best hit from the forward alignment step is used as a query protein (or query gene). Protein alignment is performed against the protein FASTA file of the Y-axis genomes. Alternatively, genes or transcripts (i.e., nucleotide sequences) can be used instead of protein sequences for the backward alignment using tools such as ggsearch or BLASTn. If the best hit from the backward alignment is the same as the query protein used in the forward alignment, then an X-axis protein (or gene) and its forward alignment hit are BBHs, and the BBH value in the table is defined as True. If the best hit from the backward alignment is different from the query protein used in the forward alignment, then an X-axis protein (or gene) and its forward alignment hit are not BBHs, and the BBH value in the table is defined as False. This binary data is stored in the BBH table with X-axis proteins as columns and Y-axis genomes as indices. Alternatively, only the forward alignment results are used instead of full BBH results.

In addition, a co-localization table is created. If, for example, BGC predictions are used to define the search region, then the cluster number is obtained, and the cluster number of each forward alignment hit from a table containing the BGC information of each genome is obtained. The values are stored in a table with X-axis proteins as columns and Y-axis genomes as indices. If the cluster number of each forward alignment hit is the same as the core synthase homolog of the genome, then the forward alignment hit is co-localized with the core synthase homolog, and the co-localization value is defined as True. Otherwise, the forward alignment hit is not co-localized with the core synthase homolog, and the co-localization value is defined as False. This binary information of co-localization is stored in the co-localization table.

Alternatively, if a custom flanking distance (i.e., proximity zone distance) is used to define the search region, then the genome location is obtained, and a table is created, which contains genome location of each forward alignment hit. The scaffold ID and the coordinates of the start and end positions of each protein are stored. A co-localization table that contains the binary information of the co-location between each forward alignment hit and a core synthase homolog of the corresponding genome is created. Co-localization value is defined as True if the query protein and the core synthase homolog are located within the specified distance (i.e., proximity zone) in the same scaffold.

A final table storing values for the cells of the heat map are created based on the BBH and co-localization tables as described above. The following transformations are applied: convert True to 1 and False to 0 from the binary data of the table containing BBH information; and convert True to 1 and False to −1 from the binary data of the table containing the co-localization information. To compute the value of each cell of the heat map, multiply sequence identity, BBH information, and the co-localization information value of all combinations with X-axis proteins and Y-axis genomes. For example, a cell may have the value of: Sequence identity (96.28)*BBH (1 or 0)*co-localization (1 or −1). If there is no BBH, the value of the cell becomes 0. If the cell corresponds to a gene not co-localized with the core synthase gene, the cell value becomes minus of the sequence identity. For the heat map based on forward alignment hits, the value of each cell is computed as the product of sequence identity and the co-localization information value of all combinations with X-axis proteins and Y-axis genomes. A heat map is plotted using the final table and the linkage matrix. A diverging colormap may be used to visualize the value from −100 to 100. The Y-axis can be reordered based on hierarchical clustering of the linkage matrix.

The generated grid representation (e.g., heat map, or data matrix) and/or subsets of the generated grid representation can be inputted into a machine-learning model, such as an LSTM, or CNN, to provide a likelihood that the putative embedded gene (e.g., an pETaG) is associated with the BGC.

### Machine-Learning Model for Performing Embeddedness Classification

At block 704 of FIG. 7, the system inputs the grid representation into a machine-learning model, in which the machine-learning model is trained to determine a likelihood that the putative embedded gene is associated with the gene cluster (e.g., a BGC) based on values of the plurality of cells in the grid representation. Non-limiting examples of machine learning models that may be used include, but are not limited to, artificial neural networks (ANNs), convolutional neural networks (CNNs) or other recurrent neural networks (RNNs), multilayer perceptrons (MLPs), deep neural networks (DNNs), LSTMs, vision transformer models, generative adversarial networks (GANs), variational autoencoders, latent diffusion models. etc.

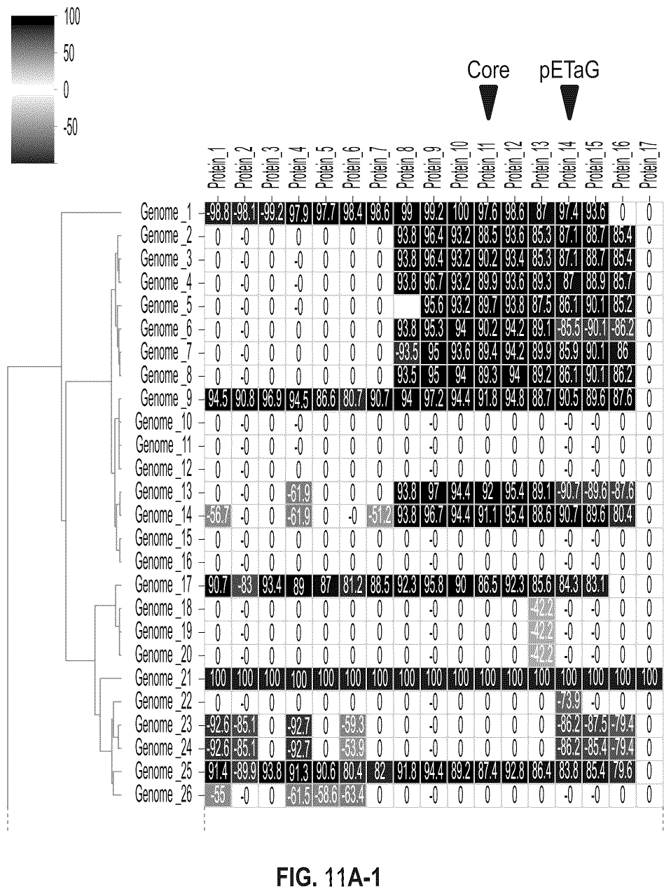

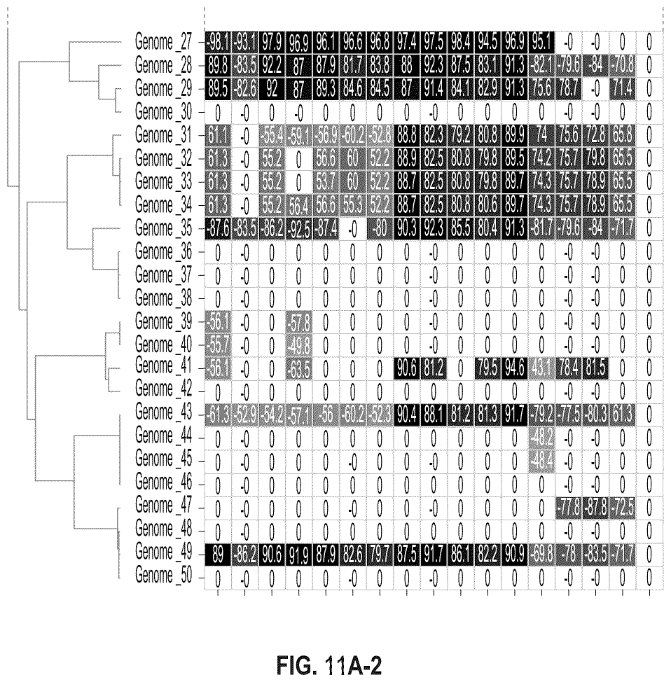

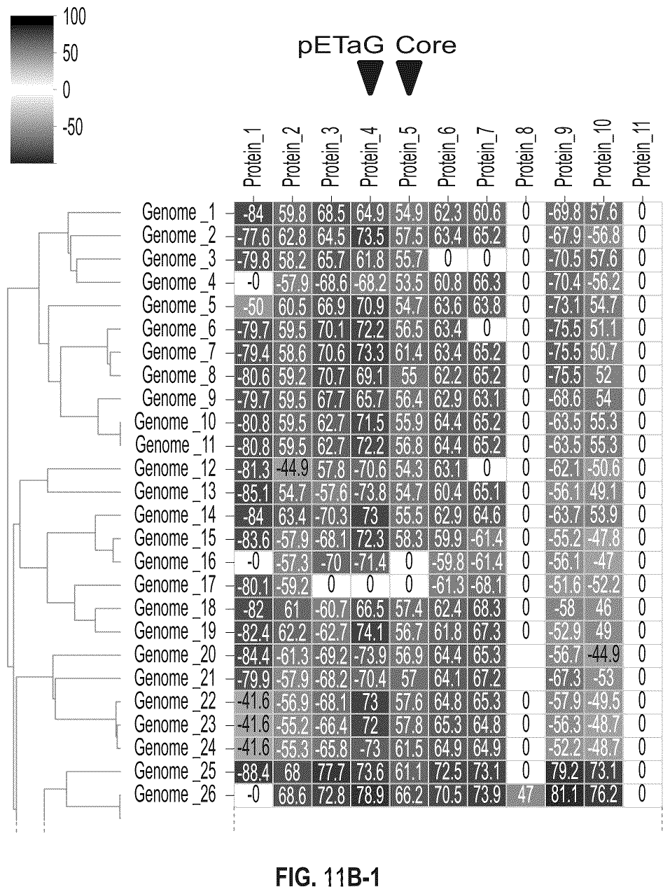

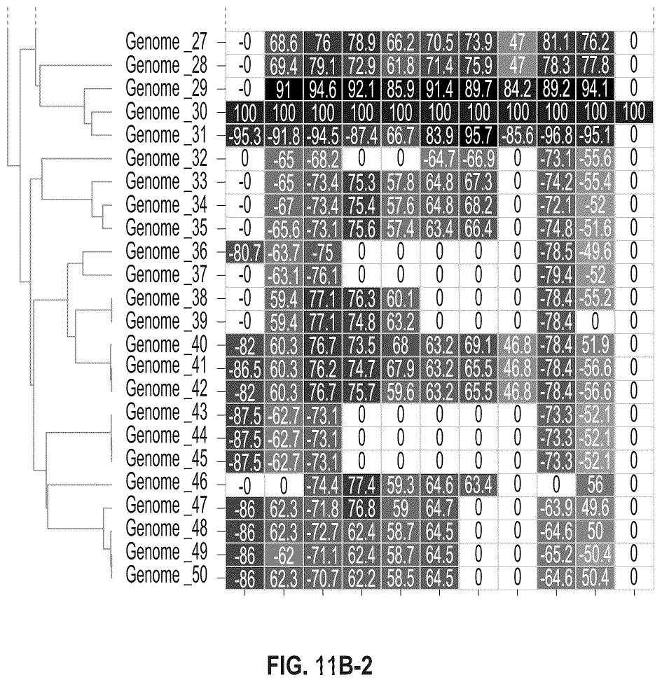

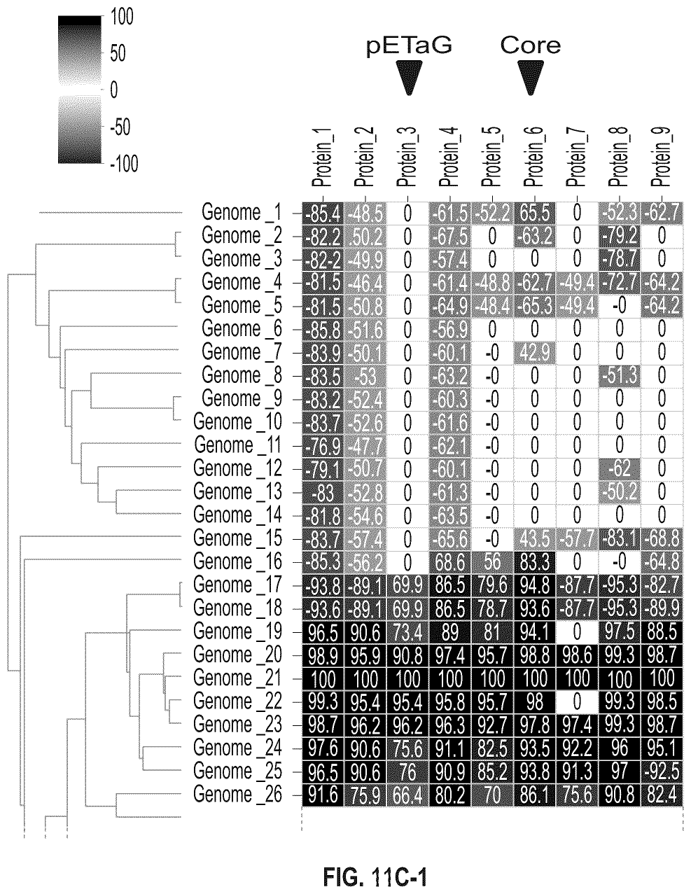

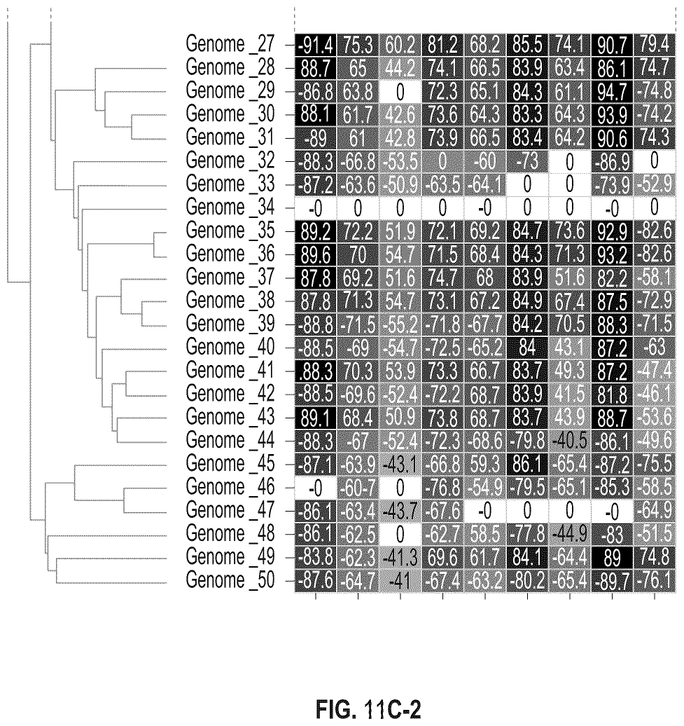

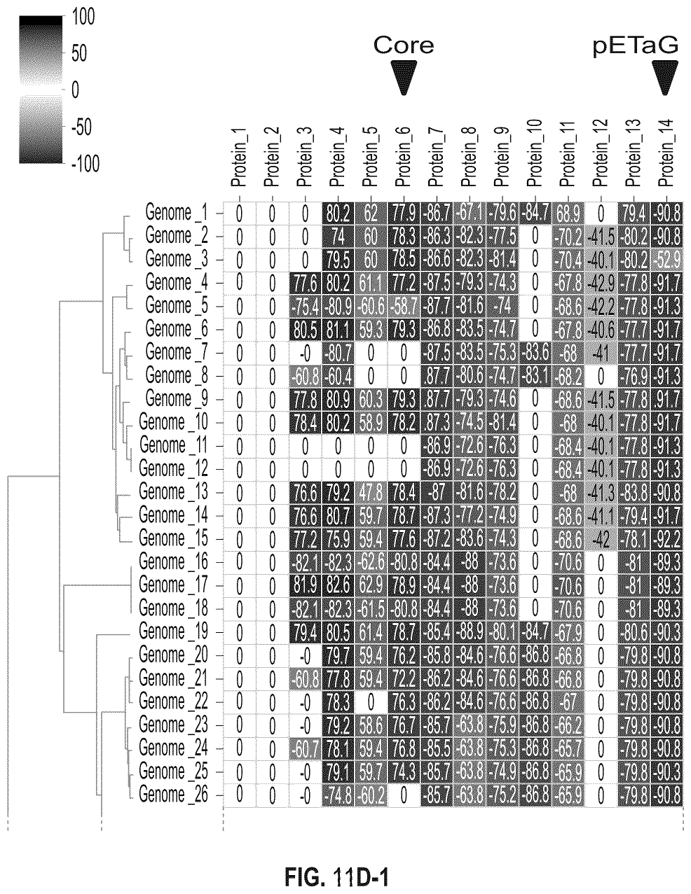

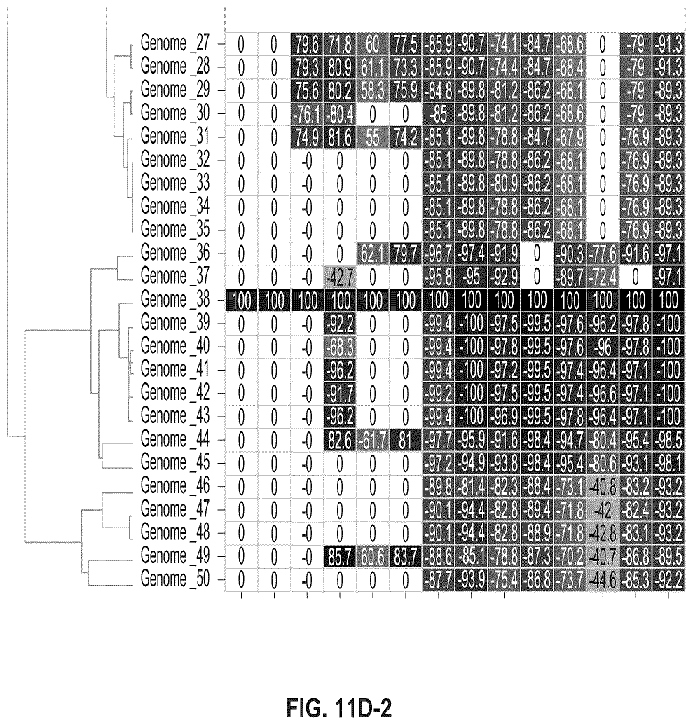

The likelihood may be a probability that the putative embedded gene is associated with the gene cluster (e.g., a BGC), or a probability that the putative embedded gene falls within one of a plurality of predefined likelihood categories. In some instances, the likelihood may fall within one of the following four categories: (1) high likelihood that the putative embedded gene is associated with the gene cluster (e.g., BGC) (also referred herein as “Tier A+”); (2) more likely than not that the putative embedded gene is associated with the gene cluster (e.g., BGC) (also referred herein as “Tier 1”); (3) more unlikely than not that the putative embedded gene is associated with the gene cluster (e.g., BGC) (also referred herein as “Tier 2”); and (4) low likelihood that the putative embedded gene is associated with the gene cluster (e.g., BGC) (also referred herein as “Tier 3”). Tier A+ heat maps have clearly defined gene cluster (e.g., BGC) boundaries, and the putative embedded gene (e.g., a putative resistance gene or pETaG) is within those boundaries. See, for example FIGS. 11A-1 and 11A-2. Tier 1 heat maps have gene cluster (e.g., BGC) boundaries that are not clearly defined, but the putative embedded gene (e.g., a putative resistance gene or pETaG) is correlated with the anchor gene (e.g., a core synthase gene). See, for example FIGS. 11B-1 and 11B-2. Tier 2 heat maps tend to provide insufficient information for identification of the gene cluster (e.g., BGC) boundaries, or allow acceptance or rejection of the correlation between the putative embedded gene (e.g., a putative resistance gene or pETaG) and the anchor gene (e.g., a core synthase gene). See, for example FIGS. 11C-1 and 11C-2. Tier 3 heat maps indicate that the putative embedded gene is a false positive because either: (1) the boundaries of the gene cluster (e.g., BGC) are clearly defined and the putative embedded gene (e.g., a putative resistance gene or pETaG) is not within the boundaries; or (2) there is no correlation or co-localization between the putative embedded gene (e.g., a putative resistance gene or pETaG) and the anchor gene (e.g., a core synthase gene). See, for example FIGS. 11D-1 and 11D-2. The method may output a probability associated with each of these likelihood categories. For example, the method may provide a probability associated with each of the four categories, in which the sum of the four probabilities is equal to 100%.

At block 706 of FIG. 7, the system obtains from the machine-learning model a likelihood that the putative embedded gene is associated with the gene cluster (e.g., BGC).

At block 708 of FIG. 7, the system displays, on a display, the grid representation (e.g., a heat map) and the likelihood that the putative embedded gene is associated with the gene cluster (e.g., BGC).

FIGS. 8A-1 to 8A-3 illustrate a heat map that represents the degree of “embeddedness” (i.e., association with a BGC) of a pETaG in the computed heat map, in accordance with the presently disclosed examples. In the heat map, the putative BGC is identified by antiSMASH in the genome marked by an asterisk (*). Each of the columns along the X-axis represents a protein (“Protein X”) encoded by a query gene in the BGC identified in the query genome marked by an asterisk (*). The genes in a BGC may be identified by antiSMASH or based on proximity (e.g., within 20 kb) to the core synthase gene of a BGC. The columns corresponding to the pETaG and the core synthase gene are marked with arrows. Each of the rows along the Y-axis represents a unique genome (“Genome Y”) selected from the genome database. Half of the genomes contain a BBH of the anchor gene, and are referred herein as “positive genomes.” Half of the genomes do not contain a BBH of the anchor gene, and are referred herein as “negative genomes.” Each cell is colored or shaded according to the presence or absence of a BBH of the respective query gene, and the percentage sequence identity (numbers in cells) of the BBH to the respective query gene. For example, cell (X,Y) is blank if there is no BBH of Protein X in Genome Y; cell (X,Y) is, e.g., blue or positive, if there is a BBH of Protein X in Genome Y and the BBH is in the same antiSMASH BGC cluster as the BBH of the core synthase gene in Genome Y; or cell (X, Y) is e.g., red or negative, if there is a BBH of Protein X in Genome Y and the BBH is not in the same antiSMASH BGC cluster as the BBH of the core synthase gene in Genome Y or if there is no BBH of the core synthase gene in Genome Y. The intensity of the red or blue color (or grayscale shading) of cell (X, Y) is based on percentage sequence identity of the BBH of Protein X in Genome Y to Protein X. The heat map is hierarchically clustered based on pairwise sequence identities among the genomes.

FIGS. 8B-1 and 8B-2 and FIGS. 8C-1 and 8C-2 illustrate an exemplary long short-term memory (LSTM) model used for classification of an input heat map into one of four tiers: (1) high likelihood that a putative embedded target gene (pETaG) is associated with the gene cluster (e.g., BGC) (“Tier A+”); (2) more likely than not that a pETaG is associated with the gene cluster (e.g., BGC) (“Tier 1”); (3) more likely than not that a pETaG is not associated with the gene cluster (e.g., BGC) (“Tier 2”); and (4) low likelihood that a pETaG is associated with the gene cluster (e.g., BGC) (“Tier 3”). In some embodiments, the heat map and/or one or more subsections thereof may be used as input for the LSTM.

As illustrated in FIGS. 8B-1 and 8B-2, the values of cells in each column of the heat map are sequentialized into a plurality of input arrays each corresponding to a query gene across a number of genomes, and each of the plurality of input arrays is inputted into an LSTM cell. Each input array also harbors the positional information of the pETaG and the core synthase, represented by two scalars, with 1 and 0 corresponding to the presence and absence of the particular gene (pETaG or core synthase). The plurality of LSTM cells provide an output tier. In some instances, subsequent to computing the heat map, vector representations of the data included in the heat map (e.g., a table of values indicating one or more patterns, one or more colors, pETaG position, and so forth) may be provided to one or more neural networks to perform an embeddedness classification of the pETaG in the heat map. For example, in some instances, the one or more neural networks may include, for example, a long short-term memory (LSTM) model, convolutional neural network, or other recurrent neural network (RNN) that may be suitable for processing genomic data or other text-based data expressed as a sequence of elements. For example, in one embodiment, the LSTM model may receive as input linear vector representations of heat map data (e.g., values indicating one or more patterns, one or more colors, pETaG position, and so forth) and output the embeddedness classification (e.g., a probability value of how embedded the pETaG is within a cluster of genes). In some instances, the LSTM model perform embeddedness classifications by classifying a pETaG into one of 4 classes: (1) “true positive” (e.g., high likelihood that a pETaG is associated with the BGC (“Tier A+”)); (2) “promising” (e.g., more likely than not that a pETaG is associated with the BGC (“Tier 1”)); (3) “inconclusive” (e.g., more likely than not that a pETaG is not associated with the BGC (“Tier 2”)); and (4) “true negative” (e.g., low likelihood that a pETaG is associated with the BGC (“Tier 3”)).

FIGS. 8B-1 and 8B-2 and FIGS. 8C-1 and 8C-2 illustrate one or more running examples of the LSTM model outputting the embeddedness classification (e.g., a probability value of how embedded the pETaG is within a cluster of genes) based on input linear vector representations of heat map data (e.g., values indicating one or more patterns, one or more colors, pETaG position, and so forth), in accordance with the presently disclosed instances. For example, in some instances, as depicted by FIGS. 8B-1 and 8B-2, the LSTM model may include a series of memory tiers, each including, for example, a respective memory cell. In some instances, the respective memory cells may operate, for example, according to its cell state (e.g., Cₜ₋₁ to Cₜ). The LSTM model may include the ability to remove or add information to the cell state, carefully regulated by structures called gates. In some instances, the gates of the respective memory cells may be provided to optionally let information through. For example, in some instances, the respective memory cells may include a sigmoid neural net layer and a pointwise multiplication operation. The sigmoid layer outputs numbers between “0” and “1”, describing how much data of each component should be passed through. For example, in one embodiment, a value of “0” means “do not let data pass,” while a value of “1” means “let data pass.” In some instances, each respective memory cell may include, for example, these gates to protect and control the cell state.

In some instances, during operation, each tier and memory cell of the LSTM model may begin by determining which of the heat map data will be discarded from the cell state. For example, in some instances, the determinations may be performed by a sigmoid layer called the forget gate layer, which looks at input data and outputs a number between “0” and “1” for each number in the cell state (e.g., Cₜ₋₁ to Cₜ) in which “1” represents completely keep this data while a “0” represents completely discard this data. The respective tiers and memory cells of the LSTM model may then determine what new information is to be stored in the cell state. For example, in some instances, a sigmoid layer called the input gate layer determines which values to update, and a tan h layer creates a vector of new candidate values that could be added to the state.

The respective tiers and memory cells of the LSTM model may then update the old cell state, Cₜ₋₁, into the new cell state Cₜ. The LSTM model may then determine what is to be output based on the cell states of each of the respective tiers and memory cells of the LSTM model. For example, in some instances, the sigmoid layer determines what parts of the cell state is to be outputted and then that cell state is passed through the tan h layer to set the values to be between “−1” and “+1” and multiply the values by the output of the sigmoid gate.

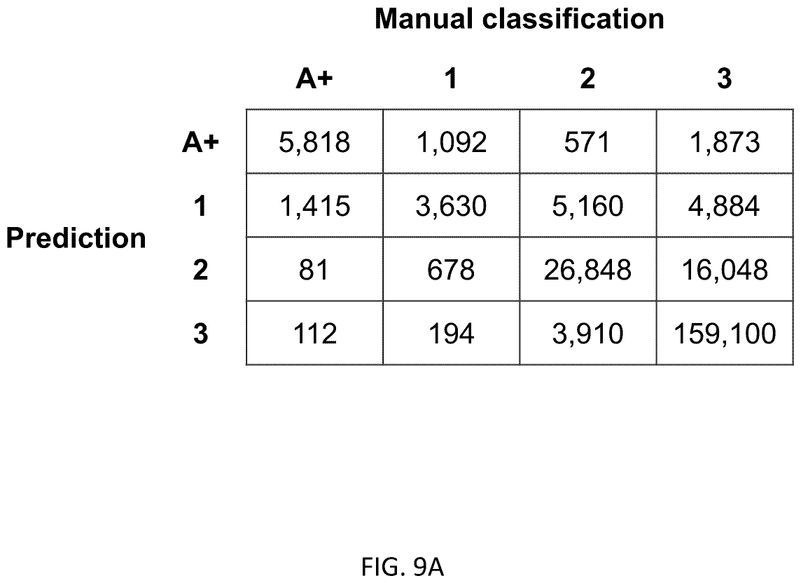

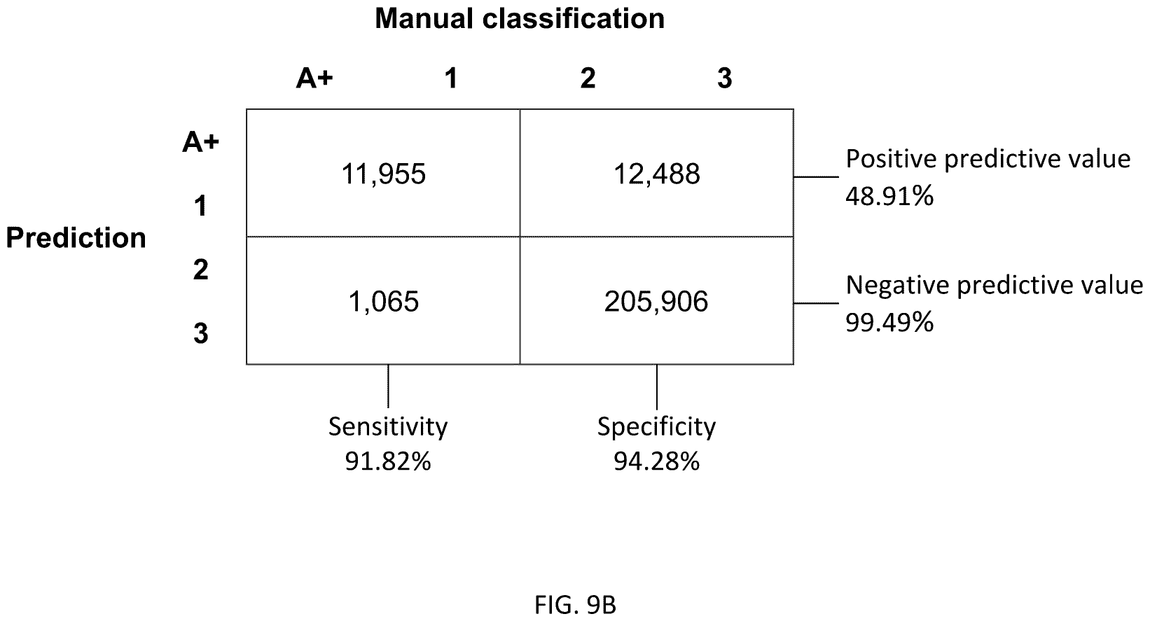

FIGS. 8C-1 and 8C-2 illustrate an example of an output of the LSTM model that, as discussed above, may represent embeddedness classifications of a pETaG into one of 4 classes: (1) “true positive” (e.g., high likelihood that a pETaG is associated with the BGC (“Tier A+”)); (2) “promising” (e.g., more likely than not that a pETaG is associated with the BGC (“Tier 1”)); (3) “inconclusive” (e.g., more likely than not that a pETaG is not associated with the BGC (“Tier 2”)); and (4) “true negative” (e.g., low likelihood that a pETaG is associated with the BGC (“Tier 3”)), as further illustrated by the prediction tables of FIGS. 9A and 9B, in accordance with the presently disclosed instances.

Specifically, FIG. 9A illustrates a table of prediction embeddedness benchmark values for “Tier A+”, “Tier 1”, “Tier 2”, and “Tier 3”. Similarly, FIG. 9B illustrates a table of final prediction embeddedness benchmark values for “Tier A+”, “Tier 1”, “Tier 2”, and “Tier 3”, including a positive predictive value (i.e., precision) of 48.91%, a negative predictive value of 99.49%, a sensitivity value of 91.82%, and a specificity value (i.e., recall) of 94.28%. The values in the table of FIG. 9A are calculated from comparing the manually annotated Tier versus the prediction results from the LSTM model. In the table of FIG. 9B, Tier A+ and Tier 1 results are combined and Tier 2 and Tier 3 results are combined. Sensitivity is calculated as the true predicted positives divided by total actual positives. Specificity is calculated as the true predicted negatives divided by the total actual negatives. Positive predictive value is calculated as the true predicted positives divided by the total predicted positives. Negative predictive value is calculated as the true predicted negatives divided by the total predicted negatives.

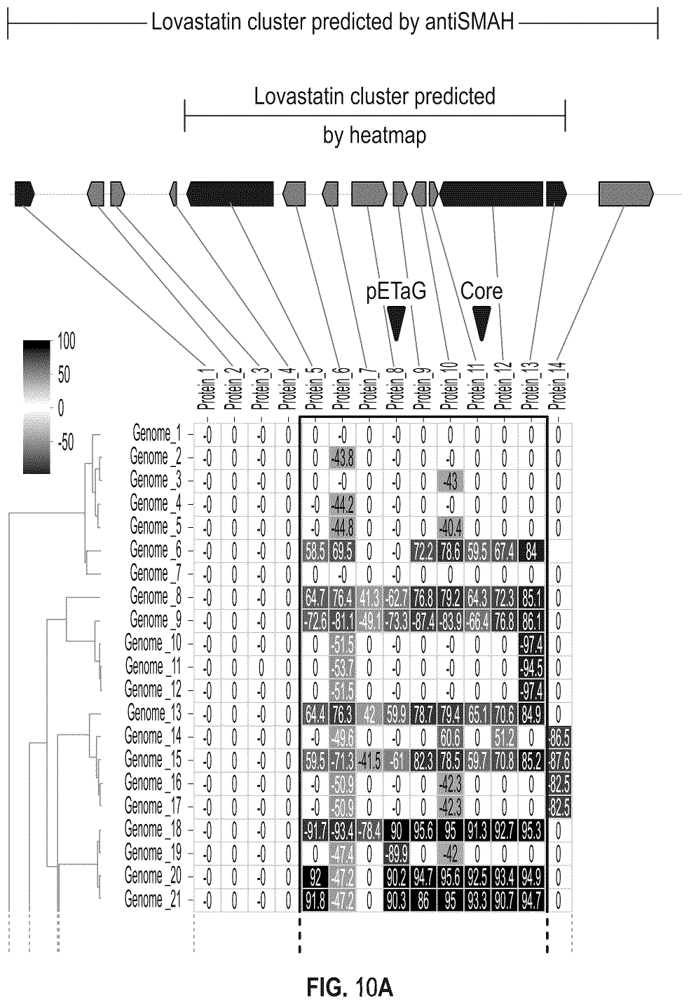

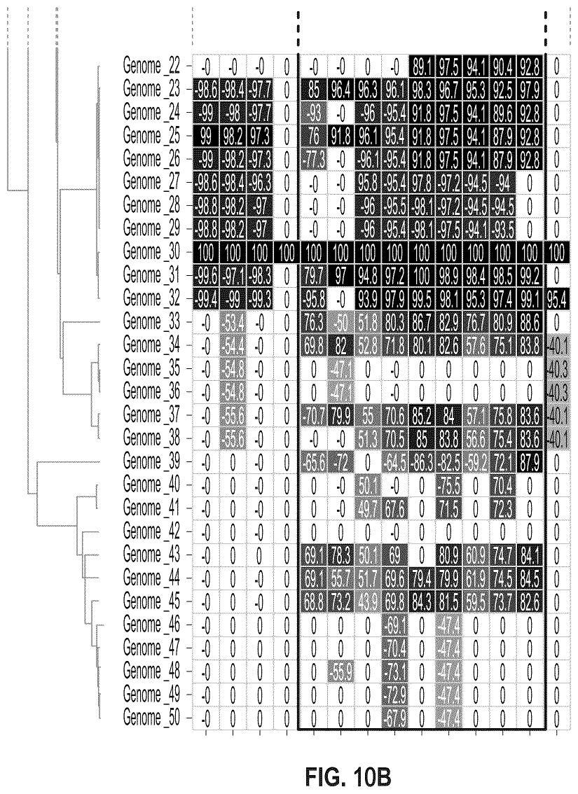

FIGS. 10A and 10B show an example of a heatmap representing a BGC for production of lovastatin as predicted by antiSMASH in Aspergillus terreus. Using the methods described herein, a smaller set of genes are identified as being associated with the BGC. FIGS. 11A-1 to 11D-2 show heat maps classified as Tier A+, Tier 1, Tier 2 and Tier 3, respectively.

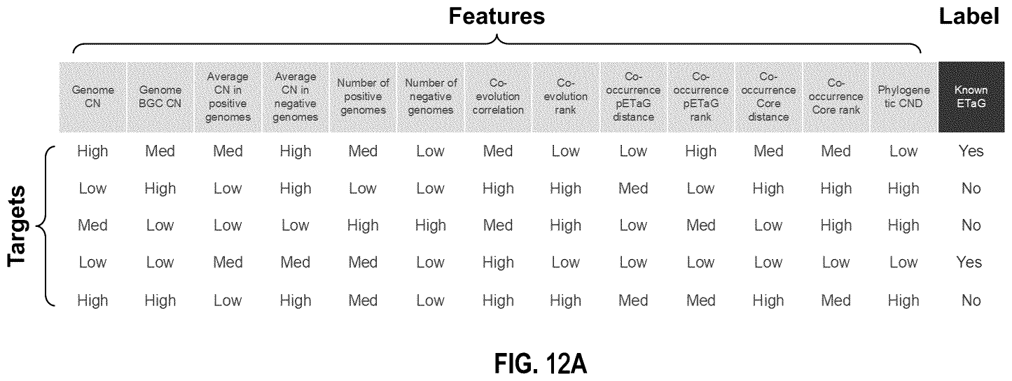

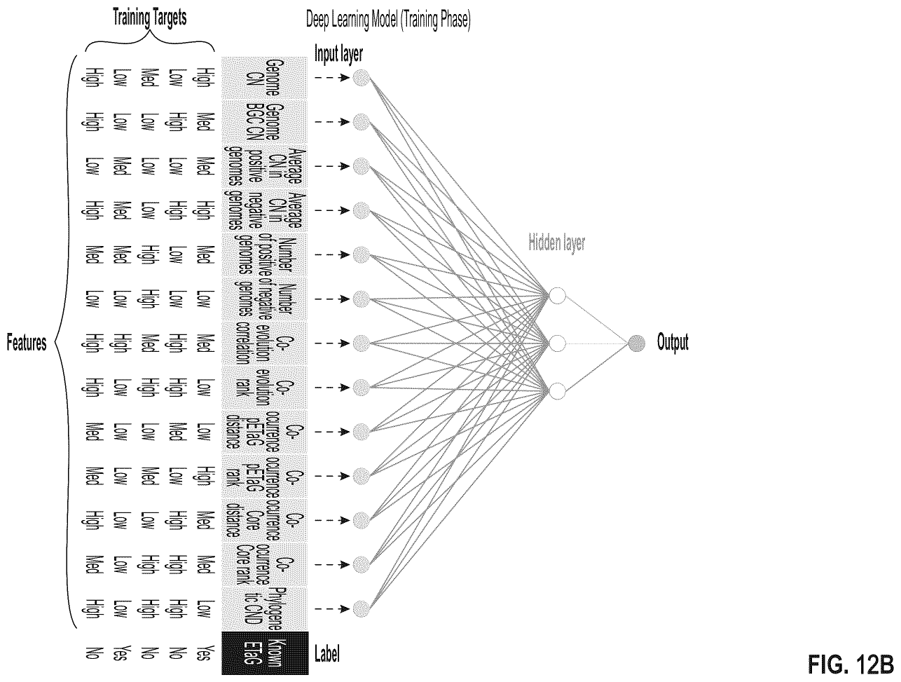

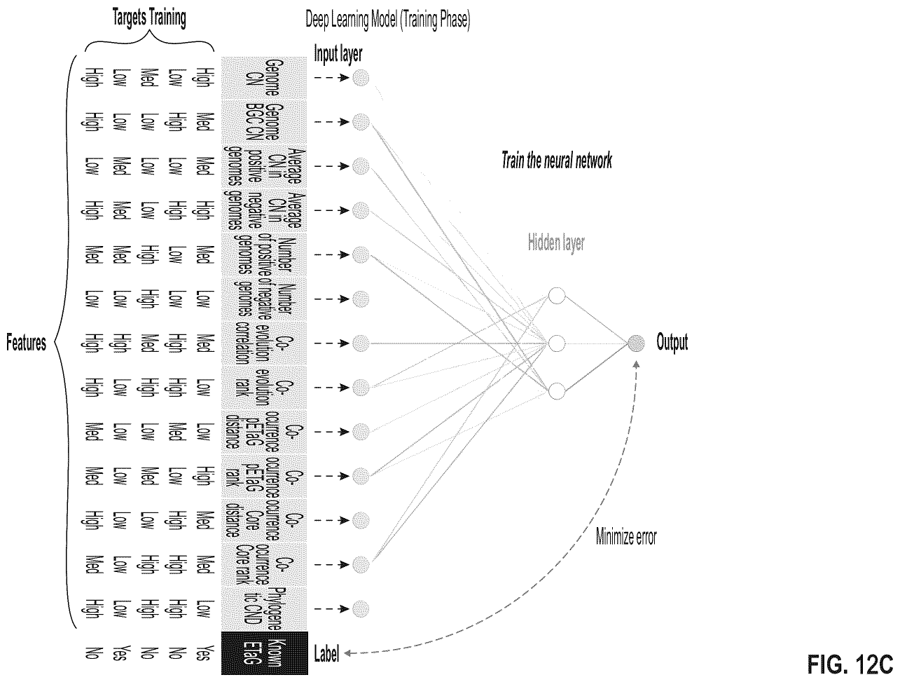

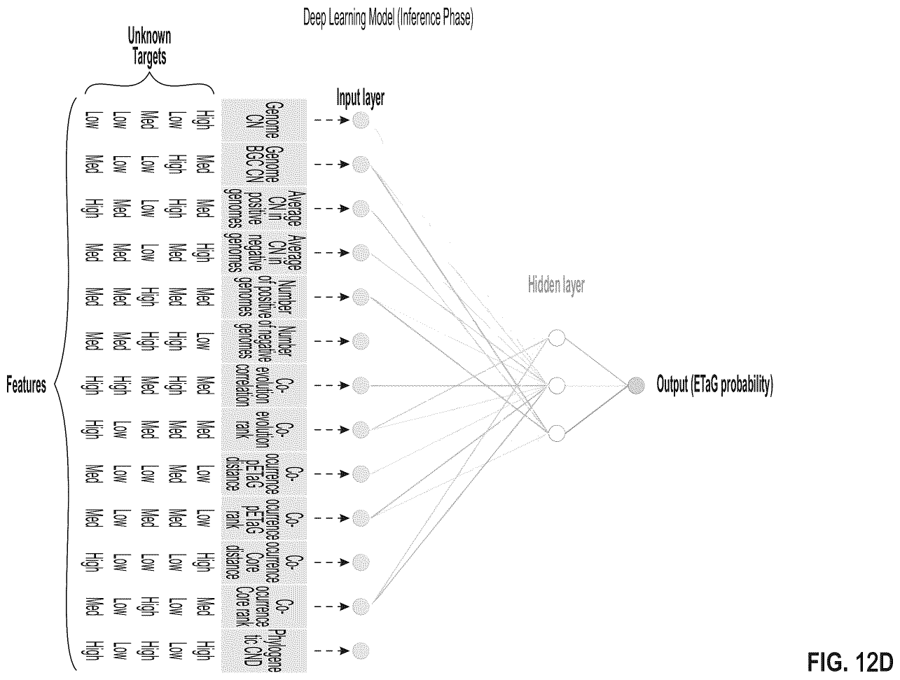

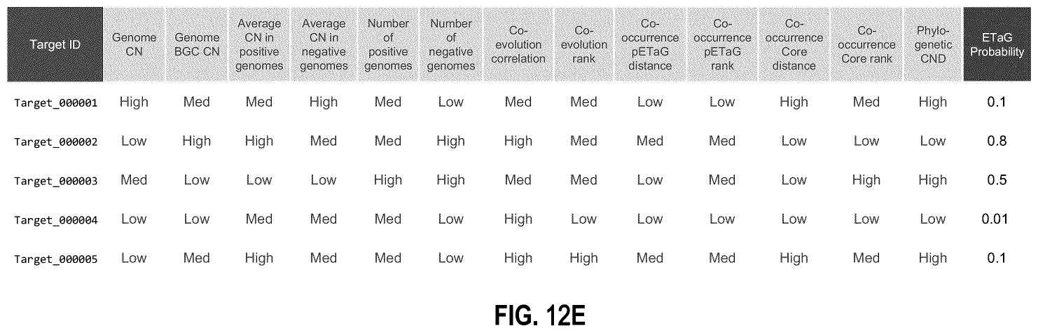

In some instances, based on the LSTM model outputting the embeddedness classification (e.g., a probability value of how embedded the pETaG is within a cluster of genes), the output(s) of the LSTM model may be organized into one or more tables (as illustrated by FIG. 12A) representing 4 features “Tier A+”, “Tier 1”, “Tier 2”, and “Tier 3”, and combined with a predetermined set of additional features (e.g., up to 27 features or more including the 4 features “Tier A+”, “Tier 1”, “Tier 2”, and “Tier 3”).

### Machine Learning Model for Predicting ETaG Likelihood

FIG. 12A illustrates a data table including a combined set of features. In some instances, the combined set of features (e.g., up to 27 features or more) may be organized into a data table as illustrated by FIG. 12A may be utilized to train a machine learning model (e.g., an artificial neural network (ANN), a multilayer perceptron (MLP), a deep neural network (DNN), a convolutional neural network, and so forth) to output an ETaG or pETaG probability value based on the inputs of the combined set of features illustrated in FIG. 12A. In some instances, the data can be utilized to train other types of machine-learning models (e.g. Bayesian inference, decision tree-based methods such as XGBoost or Random Forest, and so forth). In some instances, the data can be utilized to train logistic regression models or other types of supervised models. As further illustrated by FIG. 12A, the data table of the combined set of features may also include an annotated data set of known ETaG or pETaG label values that may be utilized, for example, as a ground truth or other reference for training the machine learning model.

FIG. 12B illustrates an initial training phase of a neural network trained to output an ETaG or pETaG probability value, in accordance with the presently disclosed instances. As illustrated, a training data set corresponding to the features included in the data table of FIG. 12A may be input to an input layer of the neural network. Due to the structured or semi-structured input data, in one embodiment, the neural network may include a multilayer perceptron (MLP) or other layered neural network including at least one hidden layer. For example, during training, the table of features may be input to respective neurons or nodes. Specifically, in some instances, the respective neurons or nodes may take as input the table of features and perform one or more designated activation functions (e.g., computational functions) to generate outputs based thereon. For example, in some instances, the designated activation functions (e.g., computational functions) may specifically determine the value of the outputs of the input neurons or nodes.

In some instances, the respective input neurons or nodes may be connected to a set of hidden neurons or nodes, which may receive the outputs of the input neurons or nodes. In some instances, the hidden neurons or nodes may constitute a hidden layer of the neural network, and may each include, for example, a weight that determine the relative strength (e.g., positive or negative) of the input of each connection to the input neurons or nodes. For example, in some instances, the hidden layer weights may influence, for example, the effect each input will have on the hidden neurons or nodes and may be adjusted iteratively for the neural network to learn over time. In some instances, as further illustrated by FIG. 12B, the neural network may be trained based on, for example, a forward propagation technique. In some instances, the forward propagation, and, by extension, the combined outputs of the hidden neurons or nodes, may include the weighted sum of the outputs of the hidden neurons or nodes and the predicted ETaG or pETaG label value (e.g., as compared to the ground truth ETaG or pETaG label value).

FIG. 12C further illustrates the training phase of a neural network trained to output an ETaG or pETaG probability value, in accordance with the presently disclosed instances. For example, as depicted by FIG. 12C, a loss function or cost function may be utilized (e.g., supervised learning) evaluate the neural network by comparing the predicted ETaG or pETaG label value to the ground truth ETaG or pETaG label value to compute a loss (e.g., error). In some instances, the weights of the hidden neurons or nodes may be iteratively adjusted, such that the loss (e.g., computed based on the comparison of the predicted ETaG or pETaG label value to the ground truth ETaG or pETaG label) may be minimized to a degree that the neural network is adequately and accurately trained.

FIG. 12D illustrates the inference phase of a neural network trained to output an ETaG or pETaG probability value, in accordance with the presently disclosed instances. As illustrated, an unknown data set of features corresponding, for example, to one or more features included in the data table of FIG. 12A, may be input to an input layer of the neural network. For example, during inference, the table of features may be input to respective neurons or nodes. Specifically, as previously discussed with respect to FIG. 12B, the respective neurons or nodes may take as input the table of features and perform one or more designated activation functions (e.g., computational functions) to generate outputs based thereon. For example, in some instances, the designated activation functions (e.g., computational functions) may specifically determine the value of the outputs of the input neurons or nodes. In some instances, the respective input neurons or nodes may be connected to a set of hidden neurons or nodes, which may receive the outputs of the input neurons or nodes. In some instances, the hidden neurons or nodes may each include, for example, a weight that determines the relative strength (e.g., positive or negative) of the input of each connection to the input neurons or nodes. In some instances, as further illustrated by FIG. 12D, the combined outputs of the hidden neurons or nodes may include the weighted sum of the outputs of the hidden neurons or nodes and the predicted ETaG or pETaG label value (e.g., as compared to the ground truth ETaG or pETaG label value). Specifically, in accordance with the presently disclosed instances, the neural network may output an ETaG or pETaG probability value based on the unknown input data set of features. FIG. 12E illustrates an example data table including the unknown input data set of features and the corresponding outputs of ETaG or pETaG probability values for each of the features, in accordance with the presently disclosed instances. In this way, the present instances may identify and determine a likelihood that an ETaG or pETaG is associated with one or more features representing BGCs.

### Applications

The computer-based methods described herein have various applications, including identification of orthologs of one or more query sequences (e.g., gene sequences of interest) in one or more query (or target) genomes, the identification of BGCs, the identification of a resistance gene against a secondary metabolite produced by a BGC in a query genome, therapeutic target identification and/or characterization, identification and/or characterization of a target of a secondary metabolite, and drug discovery, etc.

In some instances, the present disclosure provides methods and systems for determining borders of a gene cluster (e.g., a BGC) by identifying a plurality of genes associated with the gene cluster (e.g., BGC). In some instances, the method comprises: (1) identifying a plurality of query genes that are co-localized with an anchor gene (e.g., a core synthase gene) of a BGC in a query (or reference) genome; (2) for each of the plurality of query genes, performing any one of the computer-implemented methods described in the “Grid representation analysis methods” section for determining a likelihood that the query gene is associated with the BGC; and (c) identifying query genes having a specified high likelihood of being associated with the BGC as the plurality of genes associated with the BGC, wherein the specified high likelihood is a likelihood beyond a threshold value. For example, if a query gene, or an ortholog thereof, has a probability of more than any one of 30%, 40%, 50%, 60%, 70%, 80%, 90%, or higher for the category of (1) high likelihood, the query gene is associated with the BGC. In some instances, if a query gene, or an ortholog thereof, has a combined probability of more than about any one of 50%, 60%, 70%, 80%, 90%, or higher for the categories of (1) high likelihood; and (2) more likely than not, the query gene is associated with the BGC. In some instances, if a query gene, or an ortholog thereof, has a probability of more than about any one of 30%, 40%, 50%, 60%, 70%, 80%, 90%, or higher for the category of (4) low likelihood, the query gene is rejected as not being associated with the BGC. The borders (i.e., upstream and downstream limits) of the BGC can be determined based on the location of all genes associated with the BGC as determined using this method.

In some instances, the present disclosure provides methods and systems for identifying a resistance gene against a secondary metabolite produced by a BGC in a query (or reference) genome, comprising: (a) identifying a putative embedded gene that is co-localized (e.g., within a proximity zone of no more than about 100 kb, 50 kb, 20 kb, or a user-specified distance) with an anchor gene (e.g., core synthase gene) in the BGC in the query genome, wherein the putative embedded gene is not involved in the production of the secondary metabolite by the BGC; (b) performing any one of the computer-implemented methods described in the “Grid representation analysis methods” section for determining a likelihood that the putative embedded gene is associated with the BGC; and (c) identifying the putative embedded gene as a resistance gene based at least in part on the likelihood that the putative embedded gene is associated with the BGC. In some instances, the putative embedded gene is identified as a resistance gene if the likelihood that it is associated with the BGC is above a specified threshold value. In some instances, the likelihood that the putative embedded gene is associated with the BGC is one of a plurality of factors used for identifying the putative embedded gene as a resistance gene associated with the BGC. In some instances, the method further comprises experimentally verifying that the putative embedded gene is a resistance gene against the secondary metabolite produced by the BGC in the query genome. For example, the putative embedded gene may be expressed and contacted with the secondary metabolite to determine if binding occurs between the product of the putative embedded gene and the secondary metabolite.

In some instances, the present disclosure provides methods and systems for mammalian (e.g., human) target identification and/or characterization. For example, a resistance gene (e.g., a fungal resistance gene) identified using the methods described herein, which has a homolog in the human genome, provides a connection between the resistance gene, the human homolog and the secondary metabolite produced by the BGC. The connection suggests that the human homolog may be a human target of the secondary metabolite, and the secondary metabolite may interact with and/or modulate the human homolog.

In some instances, the present disclosure provides methods for identifying and/or characterizing a mammalian (e.g., human) target of a secondary metabolite of a BGC, or an analog of the BGC product, comprising: (1) identifying a putative embedded target gene (pETaG) that is co-localized (e.g., within a proximity zone of no more than about 200 kb, 100 kb, 50 kb, 40 kb, 30 kb, 20 kb or less) with the BGC in a query genome, wherein the pETaG is homologous to a mammalian (e.g., human) gene and the pETaG does not encode an enzyme that produces the secondary metabolite of the BGC; (2) performing any one of the computer-based method described in the “Grid representation analysis methods” section for determining a likelihood that the pETaG is associated with the BGC; and (3) identifying the mammalian (e.g., human) gene as a target of the secondary metabolite of the BGC based at least in part on the likelihood that the pETaG is associated with the BGC. In some instances, the mammalian (e.g., human) gene is identified as a target if the likelihood that it is associated with the BGC is above a threshold value. In some instances, the likelihood that the pETaG is associated with the BGC is one of a plurality of factors for identifying the mammalian (e.g., human) gene as a target of the secondary metabolite of the BGC. In some instances, the method further comprises identifying mammalian (e.g., human) homologs of the pETaG in a mammalian (e.g., human) genome. In some instances, the method further comprises assaying an effect of the secondary metabolite produced by the BGC, or an analog of the BGC product, on the mammalian (e.g., human) target.

In some instances, the present disclosure provides methods and systems for drug discovery, e.g., identifying a small molecule modulator of a mammalian target gene (or a reptilian target gene, avian target gene, amphibian target gene, or a target gene from any other organism). In some instances, the present disclosure provides methods for identifying a small molecule modulator of a mammalian (e.g., human) target gene or product thereof, comprising: (a) identifying a homologous gene of the mammalian target gene in a fungal genome (or in an archaea genome, a bacterial genome, a plant genome, or other genomes that comprise BGCs), wherein the homologous gene is co-localized (e.g., within a proximity zone of no more than about 100 kb, 50 kb, 40 kb, 30 kb, 20 kb or less) with an anchor gene (e.g., a core synthase gene) of a BGC of the fungal genome, and wherein the homologous gene is not involved in production of a secondary metabolite by the BGC; (b) performing any one of the computer-based methods described in the “Grid representation analysis methods” section for determining a likelihood that the homologous gene is associated with the BGC; and (c) identifying the secondary metabolite, or an analog thereof, as a small molecule modulator of the mammalian target gene, or product thereof, based at least in part on the likelihood that the homologous gene is associated with the BGC. In some instances, the secondary metabolite, or analog thereof, is identified as a small molecule modulator of the mammalian (e.g., human) gene, or product thereof, if the likelihood that the homologous gene is associated with the BGC is above a threshold value. In some instances, the likelihood that the homologous gene is associated with the BGC is one of a plurality of factors used for identifying the secondary metabolite, or an analog thereof, as a small molecule modulator of the mammalian (e.g., human) gene or product thereof. In some instances, the method further comprises assessing interactions of the mammalian target gene product with a compound derived from the secondary metabolite produced by the BGC. In some embodiments, the method comprises contacting the secondary metabolite, or analog thereof, with a protein encoded by the mammalian target gene, and detecting an activity of the protein encoded by the mammalian target gene. In some instances, the activity is binding of the protein encoded by the mammalian target gene with the secondary metabolite, or analog thereof.

In some instances, the secondary metabolite is a product of enzymes encoded by the BGC or a salt thereof, including an unnatural salt. In some instances, the secondary metabolite or analog thereof is an analog of a product of enzymes encoded by the BGC, e.g., a small molecule compound having the same core structure as the secondary metabolite, or a salt thereof.

In some instances, the present disclosure provides methods for modulating a human target, comprising: providing a secondary metabolite produced by enzymes encoded by a BGC, or an analog thereof, wherein the human target (or a nucleic acid sequence encoding the human target) is homologous to an ETaG that is associated with the BGC as determined using any one of the methods described herein.

In some instances, the present disclosure provides methods for treating a condition, disorder, or disease associated with a human target, comprising administering to a subject susceptible to, or suffering therefrom, a secondary metabolite produced by enzymes encoded by a BGC, or an analog thereof, wherein the human target (or a nucleic acid sequence encoding the human target) is homologous to an ETaG that is associated with the BGC as determined using any one of the methods described herein.

In some instances, the secondary metabolite is produced by a fungus. In some instances, the secondary metabolite is acyclic. In some instances, the secondary metabolite is a polyketide. In some instances, the secondary metabolite is a terpene compound. In some instances, the secondary metabolite is a non-ribosomally synthesized peptide.

In some instances, an analog of a substance (e.g., secondary metabolite) that shares one or more particular structural features, elements, components, or moieties with a reference substance. Typically, an analog shows significant structural similarity with the reference substance, for example sharing a core or consensus structure, but also differs in certain discrete ways. In some instances, an analog is a substance that can be generated from the reference substance, e.g., by chemical manipulation of the reference substance. In some instances, an analog is a substance that can be generated through performance of a synthetic process substantially similar to (e.g., sharing a plurality of steps with) one that generates the reference substance. In some instances, an analog is or can be generated through performance of a synthetic process different from that used to generate the reference substance. In some instances, an analog of a substance is the substance being substituted at one or more of its substitutable positions.

In some instances, an analog of a product comprises the structural core of a product. In some instances, a biosynthetic product is cyclic, e.g., monocyclic, bicyclic, or polycyclic, and the structural core of the product is or comprises the monocyclic, bicyclic, or polycyclic ring system.

In some instances, the structural core of the product comprises one ring of the bicyclic or polycyclic ring system of the product. In some instances, a product is or comprises a polypeptide, and a structural core is the backbone of the polypeptide. In some instances, a product is or comprises a polyketide, and a structural core is the backbone of the polyketide. In some instances, an analog is a substituted biosynthetic product comprising one or more suitable substituents.

### Identification of ETaGs

As noted above, in some instances the present disclosure provides methods of identifying an embedded target gene (“ETaG”) or a mammalian (e.g., human) target gene corresponding to an ETaG. The present disclosure provides methods for identifying and/or characterizing ETaGs, databases including biosynthetic gene cluster and/or ETaG gene sequences (and optionally relevant annotations), systems for identifying and/or characterizing human target genes corresponding to ETaGs, as well as methods of making and/or using such human target genes and/or systems that contain and/or express them, etc. ETaGs have been described, for example, in WO201955816A1, the contents of which are incorporated herein by reference. The methods described herein provide improved methods of identifying ETaGs that are truly associated with BGCs, and reducing calls of false positive ETaGs identified based on co-localization and/or co-regulation with one or more biosynthetic genes in a BGC in a particular genome.

In some instances, the methods described herein are applied to identify ETaGs from fungal genomes. In some instances, ETaGs from eukaryotic fungi can bear more similarities to mammalian genes than, for example, their counterparts, if any, in prokaryotes such as certain bacteria. In some instances, fungi contain ETaGs that are more therapeutically relevant, and/or contain more therapeutically relevant ETaGs, than organisms that are evolutionarily more distant from human.

In some instances, the method comprises: (a) identifying a putative ETaG (pETaG) sequence in a fungal genome, wherein: (1) the pETaG is co-localized (i.e., within a proximity zone relative) to an anchor gene (e.g., core synthase gene) in a BGC; (2) the pETaG is not involved in the production of the secondary metabolite by the BGC; and (3) the pETaG is homologous to an expressed mammalian nucleic acid sequence; (b) determining a likelihood that the pETaG is associated with the BGC using any one of the computer-based methods described in the “Grid representation analysis methods” section; and (c) identifying the pETaG as an ETaG based on the likelihood that the pETaG is associated with the BGC. For example, the pETaG may be identified as an ETaG if the likelihood is above a threshold value, or the likelihood is one of a plurality of factors used for identifying the pETaG as an ETaG. In some instances, the pETaG is co-regulated with at least one biosynthetic gene in the BGC. In some instances, the pETaG is not co-regulated with at least one biosynthetic gene in the BGC. In some instances, the method is repeated for a plurality of pETaGs and used to prioritize the pETaGs for experimental verification based on the likelihoods that the pETaGs are associated with the BGCs. In some instances, the ETaG is compared with mammalian, e.g., human, nucleic acid sequences to identify the homologous mammalian nucleic acid sequences. In some instances, such a method can be used to identify ETaGs on genomic scales, e.g., from sequences of many (e.g., hundreds, thousands, or even more) genomes. Identified ETaGs can be prioritized based on therapeutic importance of their mammalian homologs, particularly human homologs. In some instances, a biosynthetic product (i.e., secondary metabolite) produced by enzymes encoded by the related biosynthetic gene cluster, or an analog thereof, is a modulator (e.g., an activator, an inhibitor, etc.) of a human target. In some embodiments, a biosynthetic product (i.e., secondary metabolite) produced by enzymes encoded by the related biosynthetic gene cluster, or an analog thereof, is a modulator (e.g., an activator, an inhibitor, etc.) of an animal, bacterial, fungal, or plant target.

As readily appreciated by those skilled in the art, the connection between the biosynthetic products from biosynthetic gene clusters, ETaGs, and human targets, once established, can be utilized in various methods. For example, one may start from a biosynthetic product produced by the enzymes encoded by a biosynthetic gene cluster, to identify an ETaG located within a specified proximity zone of a biosynthetic gene of the biosynthetic gene cluster, and then identify a human target that is homologous to the ETaG. Once the human target is identified, one can prioritize it (even if it was previously considered undruggable), and develop modulators of the human target using the biosynthetic product, including optional further optimization of the biosynthetic product, for medical use, e.g., by preparing and assaying analogs of the product, using any of a variety of methods known to those of skill int he art. One may also start from a human target of therapeutic interest, to identify an ETaG homologous to the human target, and then to identify a biosynthetic gene cluster that contains a biosynthetic gene within a specified proximity zone of the ETaG. Once the biosynthetic gene cluster is identified, the biosynthetic product produced by the enzymes encoded by the biosynthetic gene cluster can be characterized and assayed for modulation of the human target, or a product thereof. The biosynthetic product can be used as a lead compound for optimization of drug candidates using any of a variety of methods known to those of skill in the art to provide agents useful for many medical, e.g., therapeutics purposes. In some embodiments, the target can be from other kingdoms of life such as animals, plants, fungal, bacterial, archaea.

In some instances, the present disclosure provides insights particularly into targets that were considered undruggable prior to the present disclosure, by providing methods for identifying their homologous ETaGs in fungi and elucidating the associated biosynthetic gene clusters. In some instances, the present disclosure greatly improves druggability of targets that were considered undruggable prior to the present disclosure, in some cases, essentially converting them into druggable targets, by, for example, identifying their homologous ETaGs in fungi, elucidating the associated biosynthetic gene clusters, and testing the biosynthetic products of the related biosynthetic gene clusters (which can be directly used as modulators, and/or whose analogs can be used as modulators, of the human targets).

An ETaG is within a proximity zone relative to an anchor gene (e.g., a core synthase gene) in a BGC, is homologous to an expressed mammalian nucleic acid sequence, and is optionally co-regulated with at least one biosynthetic gene in the BGC. In some instances, the ETaG is located not more than about any one of 100 kb, 50 kb, 40 kb, 30 kb, 20 kb, 10 kb, or less from an anchor gene (e.g., a core synthase gene) is a BGC.

In some instances, an ETaG is homologous to a human nucleic acid sequence that is, or encodes a product, which is an existing target of therapeutic interest. In some instances, an ETaG is homologous to a human nucleic acid sequence that is, or encodes a product, which is a novel target of therapeutic interest. In some instances, an ETaG is homologous to a human nucleic acid sequence that is, or encodes a product, which is a target considered undruggable prior to the present disclosure. In some instances, an ETaG is homologous to a human nucleic acid sequence that is, or encodes a product, which is a target considered undruggable by small molecules prior to the present disclosure.

In some instances, an ETaG sequence is homologous to an expressed mammalian nucleic acid sequence in that its sequence, or a portion thereof, is at least 20%, 30%, 40%, 50%, 60%, 70%, 80%, or 90% identical to that of an expressed mammalian nucleic acid sequence. In some instances, an ETaG sequence is homologous to a mammalian nucleic acid sequence in that mRNA produced from an ETaG, or a portion thereof, is homologous to that of a mammalian nucleic acid sequence. In some instances, a homologous portion is at least 50, 100, 150, 200, 500, 1000, 2000, 3000, or 5000 base pairs in length. In some instances, a homologous portion encodes a conserved protein, or a conserved portion of protein, such as a protein domain, a set of residues that relates to a function (e.g., interaction to another molecule (e.g., a protein, a small molecule, etc.), enzymatic activity, etc.), etc., from fungi to a mammal. In some instances, a mammalian nucleic acid, e.g., a human nucleic acid sequence, is related to a human disease, disorder, or condition. In some instances, such a human nucleic acid sequence is an existing target of therapeutic interest. In some instances, such a human nucleic acid sequence is a novel target of therapeutic interest. In some instances, such a human nucleic acid sequence is a target previously considered not susceptible to targeting by, e.g., small molecules.

In some instances, an ETaG sequence is homologous to a mammalian nucleic acid sequence in that a product encoded by an ETaG, or a portion thereof, is homologous to that encoded by a mammalian nucleic acid sequence. In some instances, an ETaG sequence is homologous to a mammalian nucleic acid sequence in that a protein encoded by an ETaG, or a portion thereof, is homologous to that encoded by a mammalian nucleic acid sequence. In some instances, an ETaG sequence is homologous to a mammalian nucleic acid sequence in that a portion of a protein encoded by an ETaG is homologous to that encoded by a mammalian nucleic acid sequence.

In some instances, a portion of a protein is a protein domain. In some instances, a protein domain is an enzymatic domain. In some instances, a protein domain interacts with one or more agents, e.g., small molecules, lipids, carbohydrates, nucleic acids, proteins, etc.

In some instances, a portion of a protein is a functional and/or structural domain that defines a protein family that the protein belongs to. Amino acid resides that within specific catalytic or structural domain defining patent families can be selected based on predictive subfamily domain architecture, and optionally verified by various assays, for use in alignment analysis of homology.

In some instances, a portion of a protein is a set of key residues, either consecutive or not consecutive, that are important for a function of a protein. In some instances, a function is an enzymatic activity, and a portion of a protein is a set of residues that are required for the activity. In some instances, a function is an enzymatic activity, and a portion of a protein is a set of residues that interact with a substrate, an intermediate, or a product. In some instances, a set of residues interact with a substrate. In some instances, a set of residues interact with an intermediate. In some instances, a set of residues interact with a product.

In some instances, a function of a protein is an interaction with one or more agents, e.g., small molecules, lipids, carbohydrates, nucleic acids, proteins, etc., and a portion of a protein is a set of residues that are required for the interaction. In some instances, a set of residues each independently contact an interacting agent. For example, in some instances, each of the residues of a set independently contacts an interacting small molecule. In some instances, a protein is a kinase and an interacting small molecule is or comprises a nucleobase, and a set of residues each independently contact the nucleobase via, e.g., hydrogen bonding, electrostatic forces, van der Waals forces, aromatic stacking, etc. In some instances, an interacting agent is another macromolecule. In some instances, an interaction agent is a nucleic acid. In some instances, a set of residues are those that contact an interacting nucleic acid, for example, those in transcription factors. In some instances, a set of residues are those that contact an interacting protein.

In some instances, a portion of a protein is or comprises an essential structural element of protein effector recruitment and/or binding, for example, based on tertiary protein structures of human targets.

Portions of proteins, such as protein domains, sets of residues responsible for biological functions, etc., can be conserved from species to species, for example, in some instances from fungi to human as illustrated in the present disclosure.

In some instances, protein homology is measured based on exact identity, e.g., the same amino acid residues at given positions. In some instances, homology is measured based on one or more properties, e.g., amino acid residues bearing one or more identical or similar properties (e.g., polar, non-polar, hydrophobic, hydrophilic, size, acidic, basic, aromatic, etc.). Exemplary methods for assessing homology are widely known in the art and can be utilized in accordance with the present disclosure, for example, MAFFT, MUSCLE, TCoffee, ClustalW, etc.

In some instances, a protein encoded by an ETaG, or a portion thereof (e.g., those described in the present disclosure), is at least 20%, 30%, 40%, 50%, 60%, 70%, 80%, 85%, 90%, 91%, 92%, 93%, 94%, 95%, 96%, 97%, 98%, or 99%, or 100% (when 100% it is identical) homologous to that encoded by a mammalian nucleic acid sequence. In some instances, a protein encoded by an ETaG, or a portion thereof, is at least 50%, 60%, 70%, 80%, 85%, 90%, 91%, 92%, 93%, 94%, 95%, 96%, 97%, 98%, or 99%, or 100% homologous to that encoded by an expressed mammalian nucleic acid sequence.

In some instances, an ETaG is co-regulated with at least one biosynthetic gene in the biosynthetic gene cluster. In some instances, an ETaG is co-regulated with two or more genes in the biosynthetic gene cluster. In some instances, an ETaG is co-regulated with the biosynthetic gene cluster in that expression of the ETaG is increased, or turned on, when a biosynthetic product produced by the enzymes encoded by the biosynthetic gene cluster (a biosynthetic product of the biosynthetic gene cluster) is produced. In some instances, an ETaG is co-regulated with the biosynthetic gene cluster in that expression of the ETaG is increased, or turned on, when level of a biosynthetic product of the biosynthetic gene cluster is increased.

In some instances, an organism comprising an ETaG comprises one or more homologous genes of the ETaG. In some instances, an ETaG gene sequence is optionally more than about 10%, 20%, 30%, 40%, 50%, 60%, 70%, 80%, 85%, 90%, 95%, or 99% homologous to one or more gene sequences in the same genome. In some instances, an ETaG gene sequence is optionally more than about 10%, 20%, 30%, 40%, 50%, 60%, 70%, 80%, 85%, 90%, 95%, or 99 homologous to 2, 3, 4, 5, 6, 7, 8, 9 or more gene sequences in the same genome. In some instances, the homology is more than 10%. In some instances, the homology is more than 20%. In some instances, the homology is more than 30%. In some instances, the homology is more than 40%. In some instances, the homology is more than 50%. In some instances, the homology is more than 60%. In some instances, the homology is more than 70%. In some instances, the homology is more than 80%. In some instances, the homology is more than 90%.

In some instances, an ETaG gene sequence is optionally no more than about 10%, 20%, 30%, 40%, 50%, 60%, 70%, 80%, 85%, 90%, 95%, or 99% identical to any expressed gene sequence in at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% fungal nucleic acid sequence in the set that is from a different fungal strain and comprises a homologous biosynthetic gene cluster. In some instances, an ETaG gene sequence is optionally no more than about 10%, 20%, 30%, 40%, 50%, 60%, 70%, 80%, 85%, 90%, 95%, or 99% identical to at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% fungal gene sequence that is within a proximity zone relative to a biosynthetic gene of a homologous biosynthetic gene cluster from a different fungal strain. In some instances, an ETaG gene sequence is optionally no more than about 10%, 20%, 30%, 40%, 50%, 60%, 70%, 80%, 85%, 90%, 95%, or 99% identical to at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% fungal gene sequence that is within a proximity zone relative to a biosynthetic gene of a homologous biosynthetic gene cluster from a different fungal strain. In some instances, an ETaG gene sequence is optionally no more than about 10%, 20%, 30%, 40%, 50%, 60%, 70%, 80%, 85%, 90%, 95%, or 99% identical to any expressed gene sequence in any fungal nucleic acid sequence in the set that is from a different fungal strain and comprises a homologous biosynthetic gene cluster. In some instances, an ETaG gene sequence is optionally no more than about 10%, 20%, 30%, 40%, 50%, 60%, 70%, 80%, 85%, 90%, 95%, or 99% identical to any expressed gene sequence that is within a proximity zone relative to a biosynthetic gene of a homologous biosynthetic gene cluster from a different fungal strain. In some instances, it is no more than about 10% identical. In some instances, it is no more than about 20% identical. In some instances, it is no more than about 30% identical. In some instances, it is no more than about 40% identical. In some instances, it is no more than about 50% identical. In some instances, it is no more than about 60% identical. In some instances, it is no more than about 70% identical. In some instances, it is no more than about 80% identical. In some instances, it is no more than about 90% identical.

In some instances, a human target gene and/or a product thereof is susceptible to modulation by a biosynthetic product, or an analog thereof, of a biosynthetic gene cluster, wherein the human target gene has its homologous ETaGs embedded within the biosynthetic gene cluster or located in a specified proximity zone relative to a biosynthetic gene of the cluster. In some instances, a protein encoded by a human target gene is susceptible to modulation by a biosynthetic product, or an analog thereof, of a biosynthetic gene cluster, wherein the human target gene has its homologous ETaGs embedded within the biosynthetic gene cluster or located in a specified proximity zone relative to a biosynthetic gene of the cluster. Thus, in some instances, the present disclosure not only provides novel human target, but also provides methods and agents for modulating such human targets. In some instances, a compound produced by the enzymes of a biosynthetic gene cluster interacts and/or modulates with a target encoded by a mammalian, e.g., human, nucleic sequence that is homologous to an ETaG related to the biosynthetic gene cluster.

In some instances, the present disclosure provides methods for assessing compounds using identified ETaGs and products encoded thereby. In some instances, the present disclosure provides a method comprising: contacting at least one test compound with a gene product encoded by an embedded target gene of a fungal nucleic acid sequence, and determining that: a level or activity of the gene product is altered when the test compound is present as compared with when it is absent; or a level or activity of the gene product is comparable to that observed when a reference agent having a known effect on the level or activity is present.

In some instances, the present disclosure provides methods for identifying and/or characterizing a mammalian, e.g., human, target of a product produced by enzymes encoded by a biosynthetic gene cluster, or an analog of the product, comprising: identifying a human homolog of an ETaG that is determined to be associated with the BGC using any one of the methods described herein; and optionally assaying an effect of the product produced by enzymes encoded by a biosynthetic gene cluster, or an analog of the product, on the target.

Additional analysis can include assessing conservation/similarity of essential structural elements of protein effector recruitment/binding, for example, based on the examination of the tertiary protein structure of the human target. For example, in some instances, aligned sequences were compared to the PDB crystal structure. In some instances, only amino acids within the specific catalytic or structural domain defining the PFAM boundaries of the ETaG/target (e.g., based on predictive subfamily domain architecture) were used in an alignment analysis. The ETaG sequences were directly compared to their human counterparts by aligning all ETaGs and human target protein(s), with their phylogenetic relationships yielding quantitative correlative data (e.g. peptide sequence similarity and/or evolutionary tree visualization) corresponding to the target protein residues within 4 Angstrom of the corresponding engaging proteins.

Without the intention to be limited by any theory, in cases where these structural motifs are conserved within fungal ETaGs, it may indicate an increased probability that the metabolite produced by the ETaG-related biosynthetic gene cluster is an effector of both fungal and human target proteins, and the metabolite produced can be a drug candidate, or a lead for drug development, toward the human target. In some instances, the above analyses are used to prioritize ETaGs and their related biosynthetic gene clusters, and metabolites produced from the biosynthetic gene clusters, with respect to targeting human targets.

### Computer Systems

In some instances, the provided computer-based methods, sequences, genomes and/or databases are embodied in a computer readable medium. In some instances, the present disclosure provides systems comprising one or more non-transitory machine-readable storage media storing data representing the provided computer-based methods, sequences, genomes and/or databases. Non-transitory machine-readable storage media suitable for embodying provided data include all forms of non-volatile storage area, including by way of example, semiconductor storage area devices, e.g., EPROM, EEPROM, and flash storage area devices; magnetic disks, e.g., internal hard disks or removable disks; magneto-optical disks; and CD-ROM and DVD-ROM disks. Among other things, provided systems can be particularly efficient due to provided sets and databases having particular structures described herein.

In some instances, the present disclosure provides computer systems that can perform the methods described herein. In some instances, the present disclosure provides computer systems adapted to perform the provided methods. In some instances, the present disclosure provides computer systems adapted to query genomes and/or genomic databases, e.g., to identify homologs of one or more query sequences. In some instances, the present disclosure provides computer systems adapted to access one or more genomic databases.

Computer systems that may be used to implement all or part of the provided methods may include various forms of digital computers. Examples of digital computers include, but are not limited to, laptops, desktops, workstations, personal digital assistants, servers, blade servers, mainframes, smart televisions and other appropriate computers. Mobile devices may be used to implement all or part of provided technologies. Mobile devices include, but are not limited to, tablet computing devices, personal digital assistants, cellular telephones, smartphones, digital cameras, digital glasses and other portable computing devices. The computing devices described herein, their connections and relationships, and their functions, are meant to be examples only, and are not meant to limit implementations of the technology.

All or part of technologies described herein and their various modifications can be implemented, at least in part, via a computer program product, e.g., a computer program tangibly embodied in one or more information carriers, e.g., in one or more tangible machine-readable storage media, for execution by, or to control the operation of, data processing apparatus, e.g., a programmable processor, a computer, or multiple computers.

A computer program for provided technologies can be written in any form of programming language, including compiled or interpreted languages, and it can be deployed in any form, including as a stand-alone program or as a module, part, subroutine, or other unit suitable for use in a computing environment. A computer program can be deployed to be executed on one computer or on multiple computers at one site or distributed across multiple sites and interconnected by a network.

Actions, e.g., associated with implementing programs and technologies, can be performed by one or more programmable processors executing one or more computer programs to perform provided technologies. All or part of the processes can be implemented as, special purpose logic circuitry, e.g., an FPGA (field programmable gate array) and/or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only storage area or a random access storage area or both. Elements of a computer (including a server) include one or more processors for executing instructions and one or more storage area devices for storing instructions and data. Generally, a computer will also include, or be operatively coupled to receive data from, or transfer data to, or both, one or more machine-readable storage media, such as mass storage devices for storing data, e.g., magnetic, magneto-optical disks, or optical disks. Non-transitory machine-readable storage media suitable for embodying computer program instructions and data include all forms of non-volatile storage area, including by way of example, semiconductor storage area devices, e.g., EPROM, EEPROM, and flash storage area devices; magnetic disks, e.g., internal hard disks or removable disks; magneto-optical disks; and CD-ROM and DVD-ROM disks.

Each computing device, such as a tablet computer, may include a hard drive for storing data and computer programs, and a processing device (e.g., a microprocessor) and memory (e.g., RAM) for executing computer programs. Each computing device may include an image capture device, such as a still camera or video camera. The image capture device may be built-in or simply accessible to the computing device.

Each computing device may include a graphics system, including a display screen. A display screen, such as an LCD or a CRT (Cathode Ray Tube) displays, to a user, images that are generated by the graphics system of the computing device. As is well known, display on a computer display (e.g., a monitor) physically transforms the computer display. For example, if the computer display is LCD-based, the orientation of liquid crystals can be changed by the application of biasing voltages in a physical transformation that is visually apparent to the user. As another example, if the computer display is a CRT, the state of a fluorescent screen can be changed by the impact of electrons in a physical transformation that is also visually apparent. Each display screen may be touch-sensitive, allowing a user to enter information onto the display screen via a virtual keyboard. On some computing devices, such as a desktop or smartphone, a physical QWERTY keyboard and scroll wheel may be provided for entering information onto the display screen. Each computing device, and computer programs executed thereon, may also be configured to accept voice commands, and to perform functions in response to such commands.

### Examples

### Example 1—Exemplary Workflow for Calculation of Phylogenetic Comparison Metrics Based on a Custom Algorithm

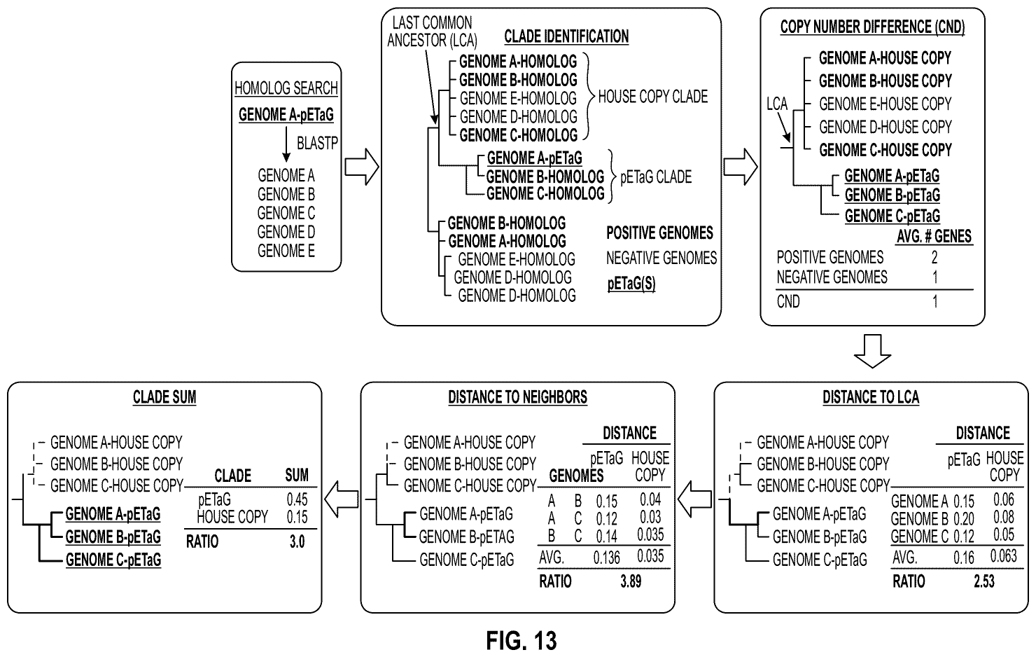

FIG. 13 provides a schematic illustration of an exemplary workflow for calculation of “phylogenetic features” using a custom algorithm. 1) A pETaG is used to search for homologs in a set of positive and negative genomes. 2) The last common ancestor (LCA) is identified, and clades are designated as pETaG or house copy clade (e.g. housekeeping version of the pETaG). 3) The copy number difference between genes in positive genomes versus negative genomes is calculated from the LCA. 4) The distance of each gene to the LCA is calculated and distances per clade are averaged and a ratio of pETaG/house copy clade is calculated. 5) The distance between pairwise combinations of genes within each pETaG or house copy clade is calculated and averaged and a ratio of pETaG/house copy clade is calculated. 6) The branch lengths of the pETaG and house copy clade are respectively summed and a ratio of pETaG/house copy clade branch lengths is calculated.

### Example 2—Phylogenetic Features Calculated for a Lovastatin ETaG

FIG. 14 provides a non-limiting example of the use of the custom phylogenetic algorithm described above on the Lovastatin ETaG. 1) A phylogenetic tree is created from a set of homologous ETaG genes from a set of positive and negative genomes. 2) The LCA clade is identified by traversing the tree clades by walking backwards from the ETaG until a clade exists that contains the ETaG and at least 1 gene from all negative genomes. 3) Clades are separated into the ETaG clade and house copy clade and the copy number difference CND is calculated between genes in positive genomes and genes in negative genomes. The results indicate that positive Lovastatin genomes have on average an increased copy number of HMG-CoA reductase homologs by 0.86. 4) Additional phylogenetic features, as described above, are calculated. The resulting ratios, being >1, indicate that the ETaG clade is faster evolving than the house copy clade.

### Example 3—Exemplary Workflow for Performing Co-Evolution Evaluation

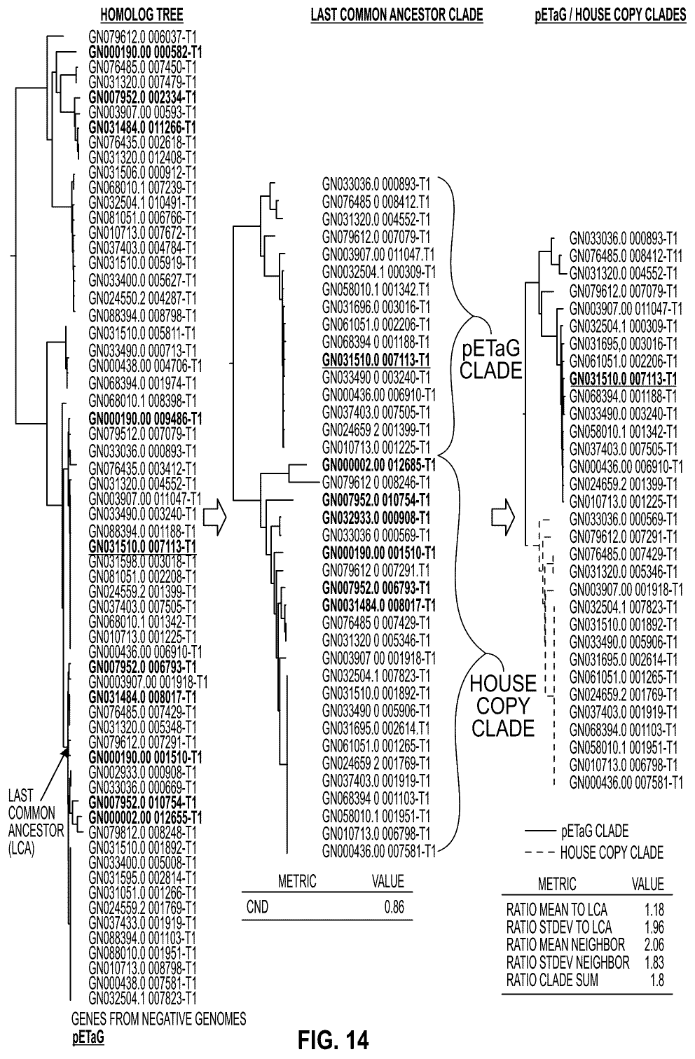

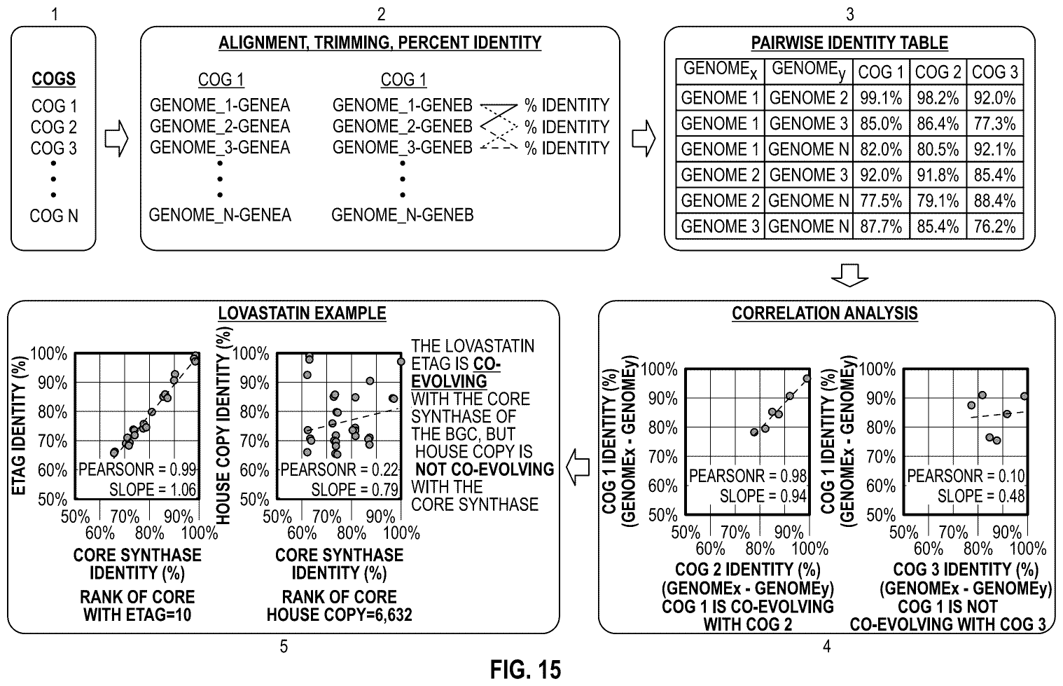

FIG. 15 provides a non-limiting example of the workflow for evaluating co-evolution where percent sequence identities were used to compare the co-evolution between pairs of COGs. 1) COGs were identified as described above. 2) After performing sequence alignment and trimming, pairwise percent sequence identities were computed for each pair of COGs. 3) Pairwise percent sequence identities were summarized in a table. Pearson R and an orthogonal regression analysis were used to examine the pairwise relationship between all pairwise combinations of COGs. 4) The plots in panel 4 provide a simulated example of the co-evolution results for three COGs—COG 1, COG 2, and COG 3. The results indicate that COG 1 is co-evolving with COG 2, but not co-evolving with COG 3. 5) The plots provided a true example of the co-evolution results for the Lovastatin ETaG (panel 5, left) and a house copy (panel 5, right) against the core synthase of the Lovastatin BGC. The results indicate that the Lovastatin ETaG is co-evolving with the core synthase, but the house copy is not.

### Example 4—Performance Data for a Deep Learning Model Trained to Evaluate pETaGs for the Likelihood that they are True ETaGs

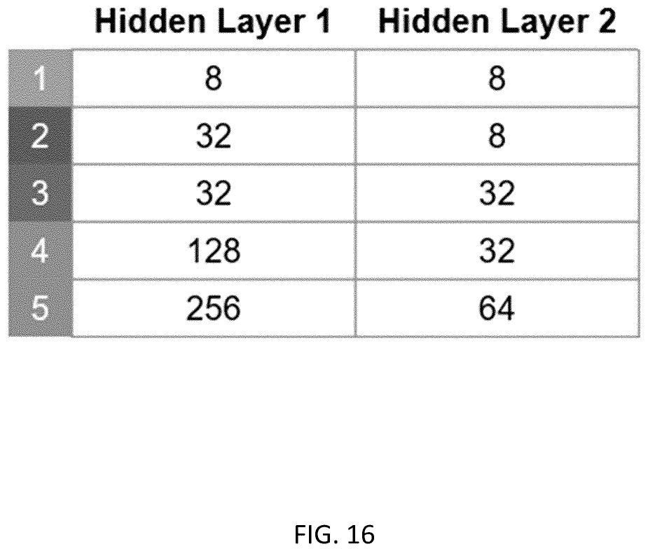

FIG. 16 provides a non-limiting example of the number of units per hidden layer in five different versions (i.e., versions 1, 2, 3, 4, and 5) of a deep learning model comprising two hidden layers trained as described above to evaluate the likelihood that a putative ETaG is an actual ETaG.

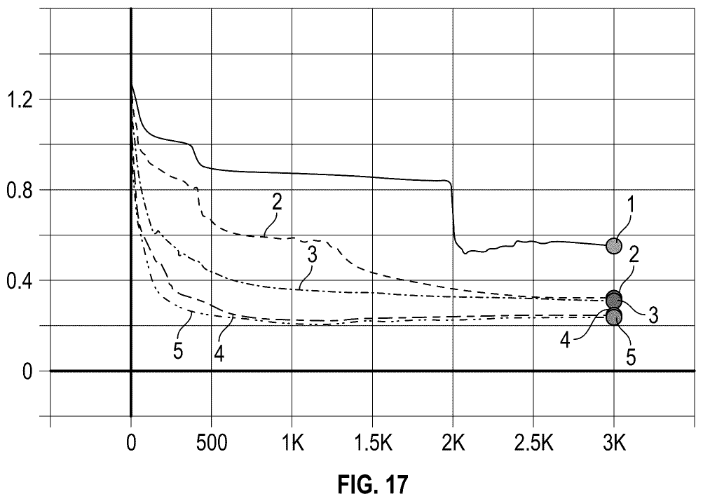

FIG. 17 provides a non-limiting example of performance data (test loss) for the five versions (i.e., versions 1, 2, 3, 4, and 5) of the deep learning model described in FIG. 16. The performance of the model, shown as a summation of the errors, after each iteration of optimization. Models that show a reduction of test loss after each iteration are determined to be a better model.

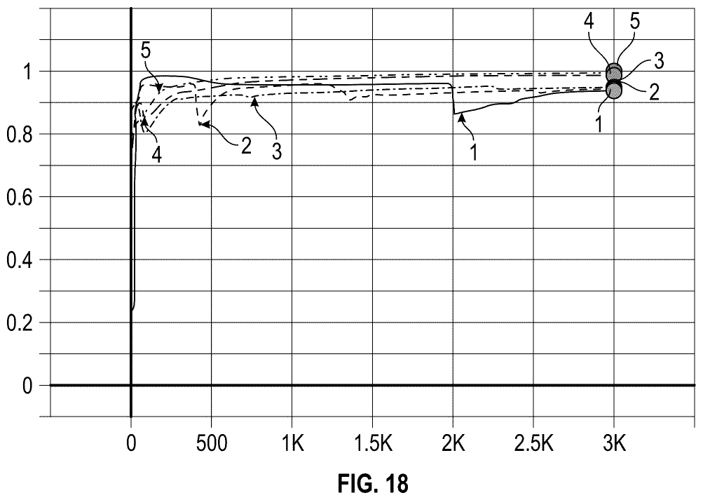

FIG. 18 provides a non-limiting example of performance data (test specificity) for the five different versions (i.e., versions 1, 2, 3, 4, and 5) of the deep learning model described in FIG. 16. i.e., the value of true predicted negatives/total actual negatives, for different training epochs.

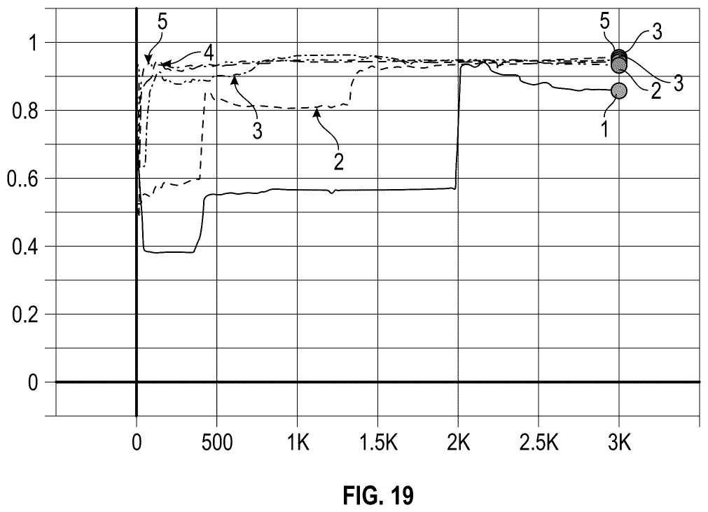

FIG. 19 provides a non-limiting example of performance data (test sensitivity) for the five different versions (i.e., versions 1, 2, 3, 4 and 5) of the deep learning model described in FIG. 16, i.e., the value of true predicted positives/total actual positives, for different training epochs.

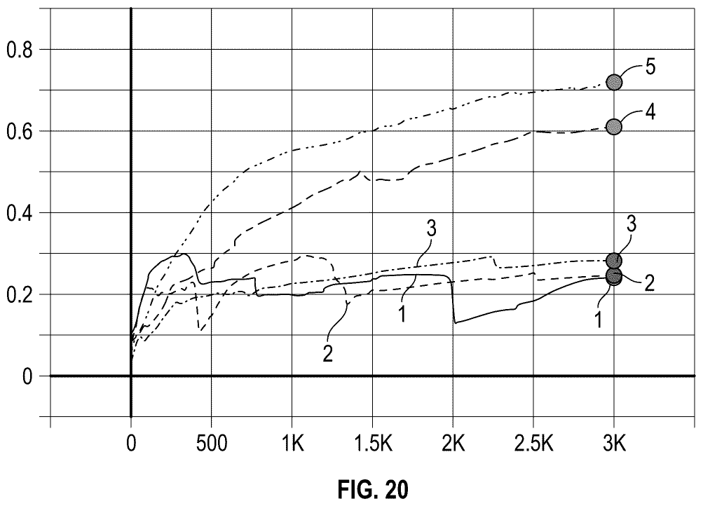

FIG. 20 provides a non-limiting example of performance data (test precision) for the five different versions (i.e., versions 1, 2, 3, 4, and 5) of the deep learning model described in FIG. 16, i.e., the value of True predicted positives/total predicted positives, for different training epochs.

### Example 5—Summary of Exemplary Target Evaluation Values for a Known ETaG Containing BGC (Lovastatin, Monascus ruber) and a Negative ETaG (Histone H3.2, Dendryphion Sp.)

Table 2 provides examples of target evaluation values for a known ETaG containing BGC (Lovastatin, Monascus ruber) and a negative ETaG (Histone H3.2, Dendryphion sp.). Two known Lovastatin candidates are shown, which had varying results from a comparative heatmap perspective. In this scenario, the empirical score and deep learning probabilities, along with the target evaluation metrics, provide confidence that the ETaG is a true ETaG.

TABLE 2

| | | | Negative |
|---|---|---|---|
| | Positive ETaG | | ETaG |
| | Lovastatin | Lovastatin | Histone H3.2 |
| | Monascus | Aspergillus | Dendryphion |
| | ruber | sp. | sp. |
| Number of pETaG-core genomes | 5 | 7 | 9 |
| Number of non-pETaG-core | | | |
| genomes | 12 | 22 | 9 |
| COG CND | 0.667 | 1 | -0.111 |
| Agnostic tree CND | 0.667 | 1 | -0.111 |
| Tier A+ probability | 0.850 | 0.035 | 0.000 |
| Tier 1 probability | 0.143 | 0.051 | 0.001 |
| Tier 2 probability | 0.006 | 0.095 | 0.292 |
| Tier 3 probability | 0.001 | 0.819 | 0.707 |
| Tier | A+ | 3 | 3 |
| PhyloCCC tree CND | 0.70 | 0.98 | 0.33 |
| Ratio mean to LCA | 1.01 | 1.38 | 2.32 |
| Ratio stdev to LCA | 2.21 | 5.86 | 68.11 |
| Ratio mean neighbor distance | 1.04 | 1.61 | 461.69 |
| Ratio stdev neighbor distance | 3.33 | 1.89 | 93.30 |
| Ratio clade sum | 1.41 | 1.52 | 1769.29 |
| Co-occurrence pETaG distance | 0.093 | 0 | 0.192 |
| Co-occurrence pETaG rank | 1 | 1 | 91 |
| Co-occurrence Core distance | 0.093 | 0.108 | 0.287 |
| Co-occurrence Core rank | 1 | 3 | 1169 |
| Co-evolution correlation | 0.824 | 0.933 | 0.442 |
| Co-evolution rank | 130 | 36 | 7167 |
| Co-evolution slope | 1.28 | 1.21 | 1.90 |
| Number positive genomes | 10 | 7 | 10 |
| PGI mean positives | 77.27 | 78.46 | 81.24 |
| PGI stdev positives | 8.41 | 7.85 | 4.67 |
| Number negative genomes | 4 | 6 | 3 |
| PGI mean negatives | 73.87 | 65.62 | 81.46 |
| PGI stdev negatives | 8.29 | 5.34 | 7.83 |
| Empirical Score | 0.75 | 0.812 | 0.125 |
| ETaG Probability | 0.998 | 1 | 0 |

### Exemplary Embodiments

Among the provided embodiments are:

1. A computer-implemented method for identifying an embedded target gene (ETaG) comprising:
specifying one or more query sequences, or proxies thereof;
selecting one or more target genomes;
performing a search of the one or more target genomes using the one or more query sequences, or proxies thereof, to identify putative embedded target gene (pETaG) sequences that are homologs of the one or more query sequences based on a comparison of one or more homologous sequence-based metrics for candidate pETaGs to one or more predetermined homologous sequence-based metric thresholds; and
determining whether or not a given pETaG is an ETaG based on a comparative genomics analysis of a plurality of genomes.
   2. The computer-implemented method of embodiment 1, wherein the comparative genomics analysis comprises generating a comparative genomics heatmap based on the plurality of genomes.
   3. The computer-implemented method of embodiments 1 or embodiment 2, wherein the plurality of genomes comprises a plurality of positive genomes and a plurality of negative genomes.
   4. The computer-implemented method of any one of embodiments 1 to 3, wherein the comparative genomics analysis comprises determining a phylogenetic feature, a co-occurrence feature, a co-evolution feature, or any combination thereof, for the given pETaG based on the plurality of genomes.
   5. The computer-implemented method of any one of embodiments 1 to 4, wherein the comparative genomics analysis comprises an analysis of an input data set comprising phylogenetic features, co-occurrence features, co-evolution features, comparative genomics heatmaps, data derived from comparative genomics heatmaps, or any combination thereof, for a pETaG using a machine learning model or an empirical algorithm to predict a probability that the pETaG is an ETaG.
   6. The computer-implemented method of any one of embodiments 1 to 5, further comprising determining that an identified pETaG is related to a resistance mechanism based on a determination of a copy number for the identified pETaG.
   7. The computer-implemented method of any one of embodiments 1 to 6, further comprising determining that a pETaG is related to a resistance mechanism based on a determination of a copy number difference between positive genomes that comprise the pETaG and negative genomes that to not comprise the pETaG.
   8. The computer-implemented method of any one of embodiments 1 to 7, wherein the one or more query sequences, or proxies thereof, comprise one or more protein sequences, one or more nucleic acid sequences, one or more Universal Protein Resource (Uniprot) identification numbers, one or more profile Hidden Markov Models (pHMMs), a specified set of protein sequence domains, or any combination thereof.
   9. The computer-implemented method of any one of embodiments 1 to 8, wherein the one or more query sequences, or proxies thereof, are selected from a bacterial genome, an archaebacterial genome, a fungal genome, a plant genome, an animal genome, a human genome, or any combination thereof.
   10. The computer-implemented method of any one of embodiments 1 to 8, wherein the one or more target genomes are selected from a bacterial genome, an archaebacterial genome, a fungal genome, a plant genome, an animal genome, a human genome, or any combination thereof.
   11. The computer-implemented method of any one of embodiments 1 to 10, wherein two or more target genomes are selected based on a pairwise similarity score, a pairwise phylogenetic distance, or any combination thereof.
   12. The computer-implemented method of embodiment 11, further comprising filtering the two or more selected target genomes to retain only those target genomes for which: (i) the pairwise similarity score is greater than a specified pairwise similarity threshold, or (ii) the pairwise phylogenetic distance is less than a specified phylogenetic distance threshold.
   13. The computer-implemented method of embodiment 12, further comprising clustering the retained target genomes into sets using a clustering algorithm, and performing the search using one or more of the clustered sets of target genomes.
   14. The computer-implemented method of embodiment 13, wherein the clustering algorithm comprises a Markov cluster algorithm.
   15. The computer-implemented method of any one of embodiments 1 to 14, wherein the search is performed using BLAST, DIAMOND, HMMER, Exonerate, or ggsearch.
   16. The computer-implemented method of any one of embodiments 1 to 15, wherein the search is restricted to one or more specific regions of the one or more target genomes.
   17. The computer-implemented method of embodiment 16, wherein the one or more specific regions comprise one or more biosynthetic gene clusters (BGCs).
   18. The computer-implemented method of embodiment 17, wherein the one or more BGCs in the one or more target genomes are predicted using a BGC search algorithm.
   19. The computer-implemented method of embodiment 18, wherein the BGC search algorithm comprises antiSMASH, SMURF, TOUCAN, or deepBGC.
   20. The computer-implemented method of embodiment 17, wherein the one or more BGCs are predicted for the one or more target genomes by extracting sequence regions of a specified length that are proximal to gene sequences that match a known biosynthetic core synthase as determined using a sequence search tool.
   21. The computer-implemented method of embodiment 20, wherein the sequence search tool comprises BLAST, DIAMOND, HMMER, Exonerate, or ggsearch.
   22. The computer-implemented method of embodiment 17, wherein the one or more BGCs are predicted for the one or more query genomes using a Hidden Markov Model (HMM) of known core synthases.
   23. The computer-implemented method of embodiment 17, wherein the one or more BGCs are predicted for the one or more target genomes based on co-localization of protein sequence domains associated with known core synthases.
   24. The computer-implemented method of any one of embodiments 1 to 23, wherein the one or more homologous sequence-based metrics comprise percent sequence identity, percent sequence coverage, E-value, bitscore, HMM score, or any combination thereof.
   25. The computer-implemented method of any one of embodiments 1 to 24, wherein the one or more predetermined homologous sequence-based metric thresholds comprise a percent sequence identity threshold, a percent sequence coverage threshold, an E-value threshold, a bitscore threshold, an HMM score threshold, or any combination thereof.
   26. The computer-implemented method of embodiment 25, wherein the one or more predetermined homologous sequence-based metric thresholds comprise a percent sequence identity threshold having a value of at least 20%, at least 30%, at least 40%, at least 50%, at least 60%, at least 70%, at least 75%, at least 80%, at least 85%, at least 90%, at least 95%, or at least 98%.
   27. The computer-implemented method of embodiment 25, wherein the one or more predetermined homologous sequence-based metric thresholds comprise a percent sequence coverage threshold having a value of at least 20%, at least 30%, at least 40%, at least 50%, at least 60%, at least 70%, at least 75%, at least 80%, at least 85%, at least 90%, at least 95%, or at least 98%.
   28. The computer-implemented method of embodiment 25, wherein the one or more predetermined homologous sequence-based metric thresholds comprise an E-value threshold having a value of less than 10, less than 9, less than 8, less than 7, less than 6, less than 5, less than 4, less than 3, less than 2, less than 1, less than 0.01, less than 0.001, less than 1e⁻¹⁰, less than 1e⁻²⁰, less than 1e⁻³⁰, less than 1e⁻⁴⁰, less than 1e⁻⁵⁰, less than 1e⁻⁶⁰, less than 1e⁻⁷⁰, less than 1e⁻⁸⁰, less than 1e⁻⁹⁰, or less than 1e⁻¹⁰⁰.
   29. The computer-implemented method of embodiment 25, wherein the one or more predetermined homologous sequence-based metric thresholds comprise a bitscore threshold having a value of at least 40, at least 50, at least 60, at least 70, at least 80, at least 90, at least 100, at least 250, at least 500, at least 1000, or at least 5000.
   30. The computer-implemented method of embodiment 25, wherein the one or more predetermined homologous sequence-based metric thresholds comprise an HMM score threshold having a value of at least 10, at least 25, at least 50, at least 100, at least 250, at least 500, at least 1000, or at least 5000.
   31. The computer-implemented method of any one of embodiments 1 to 30, wherein performing the search comprises:
converting one or more query sequences that comprise protein sequences to nucleic acid sequences;
performing a search of the one or more target genomes using the one or more query sequences that have been converted to nucleic acid sequences to identify homologous nucleic acid sequences based on a comparison of the one or more homologous sequence-based metrics for candidate pETaGs to the one or more predetermined homologous sequence-based metric thresholds; and
comparing genomic coordinates of the homologous nucleic acid sequences with genomic coordinates corresponding to predicted protein sequences in the one or more target genomes.
   32. The computer-implemented method of embodiment 31, wherein if a homologous nucleic acid sequence overlaps a nucleic acid sequence corresponding to a single predicted protein sequence, and the overlap is greater than a specified nucleic acid sequence overlap threshold, the predicted protein sequence is reported as a pETaG.
   33. The computer-implemented method of embodiment 31, wherein if a homologous nucleic acid sequence overlaps nucleic acid sequences corresponding to a plurality of predicted proteins sequences, and the overlap for each is greater than a specified nucleic acid sequence overlap threshold, only one of the predicted protein sequences is reported as a pETaG.
   34. The computer-implemented method of embodiment 33, wherein the predicted protein sequence reported as a pETaG is the predicted protein sequence for which the homologous nucleic acid sequence and the nucleic acid sequence corresponding to the predicted protein sequence exhibit the largest percent sequence identity, percent sequence coverage, E-value, or bitscore value.
   35. The computer-implemented method of embodiment 33, wherein the predicted protein sequence reported as a pETaG is the predicted protein sequence for which the homologous nucleic acid sequence and the nucleic acid sequence corresponding to the predicted protein sequence exhibit the longest overlapping sequence.
   36. The computer-implemented method of embodiment 31, wherein if a homologous nucleic acid sequence overlaps one or more nucleic acid sequences corresponding to one or more predicted proteins sequences, but the overlap for each is less than a specified nucleic acid sequence overlap threshold, the longest predicted protein sequence is reported as a pETaG.
   37. The computer-implemented method of embodiment 31, wherein if a homologous nucleic acid sequence does not overlap a nucleic acid sequence corresponding to a predicted protein sequence, the genomic coordinates of the homologous nucleic acid sequence are reported as a pETaG.
   38. The computer-implemented method of any one of embodiments 32 to 37, wherein the specified nucleic acid sequence overlap threshold has a value of at least 20%, 30%, 40%, 50%, 60%, 70%, 75%, 80%, 85%, 90%, 95%, or 98%,
   39. The computer-implemented method of any one of embodiments 2 to 38, wherein the comparative genomics heatmap generated for a given pETaG comprises a plurality of cells arranged in a grid according to a first axis and a second axis, wherein the first axis corresponds to a plurality of different target genomes, wherein the plurality of different target genomes comprises a plurality of positive genomes each having an ortholog of an anchor gene sequence of a known BGC in one of the target genomes and a plurality of negative genomes that do not have an ortholog of the anchor gene sequence, wherein the second axis corresponds to a plurality of target gene sequences that are co-localized with the anchor gene sequence of the known BGC, wherein the putative embedded target gene (pETaG) is one of the plurality of co-localized target gene sequences, and wherein a numerical value for each cell is based on:
(i) a presence or absence of an ortholog of the respective co-localized query gene sequence in the respective target genome;
(ii) a sequence similarity of the ortholog to the respective co-localized query gene sequence; and
(iii) whether the ortholog of the respective query gene sequence is co-localized with the ortholog of the anchor gene sequence in the respective genome.
   40. The computer-implemented method of embodiment 39, further comprising analyzing the comparative genomics heatmap, or underlying data thereof, using a trained machine learning model, wherein the machine-learning model is trained to determine a likelihood that a putative embedded gene is embedded in a gene cluster based on the numerical values in the plurality of cells in the grid representation.
   41. The computer-implemented method of embodiment 40, wherein the trained machine learning model comprises a long short-term memory (LSTM) model or convolutional neural network (CNN).
   42. The computer-implemented method of any one of embodiments 5 to 41, wherein the machine learning model used to predict a probability that the pETaG is an ETaG comprises a supervised learning model.
   43. The computer-implemented method of embodiment 42, wherein the supervised learning model comprises a deep learning model.
   44. The computer-implemented method of embodiment 42, wherein the supervised learning model comprises a decision tree model.45. A computer-implemented method of determining a likelihood that a putative embedded target gene (pETaG) is a resistance gene against a secondary metabolite produced by a biosynthetic gene cluster (BGC) in a query genome, the method comprising:
a) determining one or more parameters selected from the following:
   i) a likelihood that the pETaG is associated with the BGC based on presence or absence of orthologs of each of a plurality of query genes that are co-localized with the BGC in a plurality of different genomes, wherein the plurality of genomes comprises a plurality of positive genomes comprising an ortholog of an anchor gene of the BGC, and a plurality of negative genomes that do not comprise an ortholog of the anchor gene of the BGC, wherein the anchor gene is known to associate with the BGC;
   ii) one or more phylogenetic features of the last common ancestor (LCA) of homologs of the pETaG in a phylogenetic tree of the plurality of genomes;
   iii) one or more scores indicative of co-occurrence of the ortholog of the pETaG and the ortholog of the anchor gene among the plurality of positive genomes;
   iv) one or more scores indicative of co-evolution of sequence divergence among orthologs of the pETaG with respect to sequence divergence among orthologs of the anchor gene in positive genomes comprising both an ortholog of the pETaG and an ortholog of the anchor gene; and
   v) one or more scores indicative of copy number of homologs of the pETaG in the plurality of positive genomes and copy number of homologs of the pETaG in the plurality of negative genomes; and
   b) determining the likelihood that the pETaG is a resistance gene against the secondary metabolite produced by the BGC based on the one or more parameters.
      46. The computer-implemented method of embodiment 45, wherein the pETaG is co-localized with the BGC in the query genome.
      47. The computer-implemented method of embodiment 46, wherein the pETaG is not involved in production of the secondary metabolite by the BGC.
      48. The computer-implemented method of any one of embodiments 45 to 47, wherein the anchor gene is the core synthase gene of the BGC.
      49. The computer-implemented method of any one of embodiments 45 to 48, wherein the method comprises determining a likelihood for each of a plurality of pETaGs that the pETaG is a resistance gene against a secondary metabolite produced by a BGC in a target genome.
      50. The computer-implemented method of embodiment 49, comprising:
a) identifying putative BGCs in a plurality of genomes having pairwise sequence similarity above a threshold value;
b) identifying non-biosynthetic genes that are co-localized with orthologs of the anchor gene in the putative BGCs, wherein the non-biosynthetic genes are homologous to any one of a plurality of query genes in an organism of interest, and wherein the non-biosynthetic genes are not involved in production of secondary metabolites by the BGCs;
c) for each of the plurality of query genes, identifying the non-biosynthetic gene encoding a protein having the highest sequence similarity to the protein of the respective target gene as the pETaG and the genome encoding the non-biosynthetic gene as the target genome; and
d) for each of the plurality of query genes, determining a likelihood that the respective pETaG is a resistance gene against a secondary metabolite produced by the respective BGC in the respective target genome.
   51. The computer-implemented method of embodiment 49, comprising:
a) clustering genomes in a database into a plurality of clusters each comprising genomes having pairwise sequence similarity above a threshold value;
b) for each of the plurality of clusters:
   i) identifying non-biosynthetic genes that are co-localized with orthologs of the anchor gene in the putative BGCs, wherein the non-biosynthetic genes are homologous to any one of a plurality of query genes in an organism of interest, and wherein the non-biosynthetic genes are not involved in production of secondary metabolites by the BGCs;
   ii) for each of the plurality of query genes, identifying the non-biosynthetic gene encoding a protein having the highest sequence similarity to the protein of the respective query gene as a candidate pETaG; and
c) clustering the candidate pETaGs into a plurality of clusters based on sequence similarities among the pETaGs, and identifying the candidate pETaG encoding a protein having the highest sequence similarity to the protein of the respective query gene in each cluster as the pETaG and the respective genome encoding the pETaG as the target genome; and
d) for each of the plurality of query genes, determining a likelihood that the respective pETaG is a resistance gene against a secondary metabolite produced by the respective BGC in the respective target genome.
   52. The computer-implemented method of embodiment 50 or embodiment 51, wherein the threshold value is at least 70%, at least 75%, at least 80%, at least 85%, at least 90%, at least 95%, or at least at least 98% pairwise sequence similarity.
   53. The computer-implemented method of any one of embodiments 50 to 52, wherein each of the identified non-biosynthetic genes encodes a protein having at least about 30% sequence identity to a protein encoded by the respective query gene.
   54. The computer-implemented method of any one of embodiments 50 to 53, wherein the plurality of query genes are all protein-coding genes in the organism of interest.
   55. The computer-implemented method of any one of embodiments 50 to 54, wherein the organism of interest is a mammal.
   56. The computer-implemented method of embodiment 55, wherein the organism of interest is human.
   57. The computer-implemented method of any one of embodiments 50 to 54, wherein the organism of interest is a reptile, a bird, an amphibian, an animal, a plant, a fungus, or a bacterium.
   58. The computer-implemented method of embodiment 55 or embodiment 56, wherein the plurality of genomes are fungal genomes.
   59. The computer-implemented method of embodiment 55 or embodiment 56, wherein the plurality of genomes are bacterial genomes.
   60. The computer-implemented method of embodiment 55 or embodiment 56, wherein the plurality of genomes are plant genomes.
   61. The computer-implemented method of any one of embodiments 54 to 60, wherein each of the plurality of clusters comprise about 10 to about 100 genomes.
   62. A computer-implemented method of identifying a druggable target in an organism of interest, comprising performing the method of any one of embodiments 45 to 61, and identifying a query gene as a druggable target based on the likelihood that the respective pETaG of the query gene is a resistance gene against a secondary metabolite produced by the BGC in the target genome.
   63. The computer-implemented method of embodiment 62, further comprising identifying the secondary metabolite or an analog thereof as a small molecule modulator of the query gene or a protein encoded by the query gene.
   64. The computer-implemented method of embodiment 63, further comprising contacting the secondary metabolite or analog thereof with a protein encoded by the query gene, and detecting an activity of the protein encoded by the query gene.
   65. The computer-implemented method of any one of embodiments 45 to 64, wherein the number of the positive genomes is equal to the number of the negative genomes.
   66. The computer-implemented method of embodiment 65, comprising selecting a plurality of positive genomes and a plurality of negative genomes from a database of genomes.
   67. The computer-implemented method of embodiment 66, comprising clustering the database of genomes into a plurality of clusters based on sequence similarities, and selecting one positive genome per cluster to provide the plurality of positive genomes.
   68. The computer-implemented method of embodiment 67, comprising selecting a negative genome that has highest sequence similarity to each positive genome in the cluster.
   69. The computer-implemented method of any one of embodiments 66 to 68, wherein the average pairwise percentage sequence identities of orthologs of one or more single copy genes in the positive genomes is no more than about 95%, and/or the average pairwise percentage sequence identities of orthologs of one or more single copy genes in the negative genomes is no more than about 95%,
   70. The computer-implemented method of any one of embodiments 45 to 69, wherein the number of the positive genomes is at least 5.
   71. The computer-implemented method of any one of embodiments 45 to 70, wherein the one or more parameters comprises a likelihood that the pETaG is associated with the BGC based on presence or absence of orthologs of each of a plurality of query genes in the BGC in a plurality of different genomes.
   72. The computer-implemented method of embodiment 71, wherein determining a likelihood that the pETaG is associated with the BGC comprises:
a) receiving a grid representation comprising a plurality of cells arranged according to a first axis and a second axis, wherein the first axis corresponds to the plurality of genomes, wherein the second axis corresponds to the plurality of query genes in the BGC in the query genome, and wherein each cell is based on:
i) the presence or absence of an ortholog of the respective query gene in the respective genome;
ii) sequence similarity of the ortholog to the respective query gene; and
iii) whether the ortholog of the respective query gene is co-localized with the ortholog of the anchor gene in the respective genome; and
b) inputting the grid representation into a machine-learning model, wherein the machine-learning model is trained to determine a likelihood that the pETaG is associated with the BGC based on values of the plurality of cells in the grid representation, thereby providing the likelihood that the pETaG is associated with the BGC.
   73. The computer-implemented method of embodiment 72, further comprising:
a) identifying a putative BGC comprising a pETaG from a library of putative BGCs from a plurality of genomes and identifying the longest biosynthetic gene in the putative BGC as the core synthase gene;
b) obtaining a plurality of positive genomes comprising an ortholog of the core synthase gene and a plurality of negative genomes that do not comprise an ortholog of the core synthase gene, wherein the plurality of positive genomes have pairwise sequence similarities no more than a threshold value, and wherein the plurality of negative genomes are selected based on sequence similarity to the plurality of positive genomes; and
c) creating a grid representation comprising a plurality of cells arranged according to a first axis and a second axis, wherein the first axis corresponds to all protein-coding genes that are co-localized with the core synthase gene in the putative BGC in the query genome, and the second axis corresponds to the plurality of positive genomes and the plurality of negative genomes, wherein each cell is computed based on:
   i) the presence or absence of an ortholog of the respective protein-coding gene in the respective genome;
   ii) sequence similarity of the ortholog to the respective protein-coding gene; and
   iii) whether the ortholog of the respective protein-coding gene is co-localized with the ortholog of the core synthase gene in the respective genome.
      74. The computer-implemented method of embodiment 72 or embodiment 73, wherein the machine-learning model is a classification model configured to output a probability for each of a plurality of predefined likelihood categories.
      75. The computer-implemented method of embodiment 74, wherein the classification model is a long short-term memory (LSTM) model.
      76. The computer-implemented method of embodiment 74, wherein the classification model is a convolutional neural network (CNN).
      77. The computer-implemented method of embodiment 74, wherein the classification model is an artificial neural network (ANN), a multilayer perceptron (MLP), a deep neural network (DNN), a vision transformer model, a generative adversarial network (GAN) model, a variational autoencoder model, or a latent diffusion model.
      78. The computer-implemented method of any one of embodiments 74 to 77, wherein the plurality of predefined likelihood categories comprise: (1) high likelihood; (2) more likely than not; (3) more unlikely than not; and (4) low likelihood.
      79. The computer-implemented method of any one of embodiments 45 to 78, wherein the one or more parameters comprises one or more phylogenetic features of the last common ancestor (LCA) of homologs of the pETaG in a phylogenetic tree of the plurality of positive and negative genomes.
      80. The computer-implemented method of embodiment 79, wherein the one or more phylogenetic features are selected from the group consisting of average copy number difference (CND) between genes in the plurality of positive genomes and genes in the plurality of negative genomes and values determined from a plurality of positive genomes, ratio mean to LCA, ratio standard deviation to LCA, mean ratio of neighbor distance, standard deviation of ratio of neighbor distance, and sum of ratio clade.
      81. The computer-implemented method of any one of embodiments 45 to 80, wherein the one or more parameters comprise one or more scores indicative of co-occurrence of the ortholog of the pETaG and the ortholog of the anchor gene among the plurality of positive genomes.
      82. The computer-implemented method of embodiment 81, wherein the one or more scores indicative of co-occurrence are selected from the group consisting of co-occurrence pETaG distance, co-occurrence pETaG rank, co-occurrence core distance, and co-occurrence core rank.
      83. The computer-implemented method of any one of embodiments 45 to 82, wherein the one or more parameters comprise one or more scores indicative of co-evolution of sequence divergence among orthologs of the pETaG with respect to sequence divergence among orthologs of the anchor gene in positive genomes comprising both an ortholog of the pETaG and an ortholog of the anchor gene.
      84. The computer-implemented method of embodiment 83, wherein the one or more scores indicative of co-evolution are selected from the group consisting of co-evolution correlation, co-evolution rank, and co-evolution slope.
      85. The method of any one of embodiments 79 to 84, wherein the one or more parameters further comprise one or more features of the plurality of positive genomes and the plurality of negative genomes.
      86. The computer-implemented method of embodiment 85, wherein the one or more features are selected from the group consisting of the number of the positive genomes, mean pairwise genomic identity (PGI) among the positive genomes, standard deviation of PGI among the positive genomes, the number of the negative genomes, mean PGI among the negative genomes, and standard deviation of PGI among the negative genomes.
      87. The computer-implemented method of any one of embodiments 45 to 86, wherein said determining the likelihood based on the one or more parameters comprises inputting the one or more features into a machine-learning model, wherein the machine-learning model has been trained to determine a likelihood that the pETaG is a resistance gene.
      88. The computer-implemented method of embodiment 87, wherein the machine-learning model is a deep learning model.
      89. The computer-implemented method of embodiment 87, wherein the machine-learning model is a decision tree model.
      90. The computer-implemented method of embodiment 87, wherein the machine-learning model is a Bayesian inference model.
      91. The computer-implemented method of embodiment 87, wherein the machine-learning model is a logistic regression model.
      92. The computer-implemented method of any one of embodiments 45 to 91, wherein whether a gene is co-localized with an anchor gene of a BGC is determined using antiSMASH.
      93. The computer-implemented method of any one of embodiments 45 to 92, wherein whether a gene is co-localized with an anchor gene of a BGC is determined based on whether the gene is located within a proximity distance from the anchor gene.
      94. The computer-implemented method of embodiment 93, wherein the proximity zone is no more than about 50 kb.
      95. The computer-implemented method of embodiment 93, wherein the proximity zone is about 20 kb.
      96. A system comprising:
one or more processors; and
a memory communicatively coupled to the one or more processors and configured to store instructions that, when executed by the one or more processors, cause the system to:
   i) receive as input one or more query sequences, or proxies thereof;
   ii) receive as input a selection of one or more target genomes;
   iii) perform a search of the one or more target genomes using the one or more query sequences, or proxies thereof, to identify putative embedded target gene (pETaG) sequences that are homologs of the one or more query sequences based on a comparison of one or more homologous sequence-based metrics for candidate pETaGs to one or more predetermined homologous sequence-based metric thresholds; and
   iv) determine whether or not a given pETaG is an actual ETaG based on a comparative genomics analysis of a plurality of genomes related to the one or more target genomes.
      97. A system comprising:
one or more processors; and
a memory communicatively coupled to the one or more processors and configured to store instructions that, when executed by the one or more processors, cause the system to perform a method of determining a likelihood that a putative embedded target gene (pETaG) is a resistance gene against a secondary metabolite produced by a biosynthetic gene cluster (BGC) in a query genome, the method comprising:
a) determining one or more parameters selected from the following:
i) a likelihood that the pETaG is associated with the BGC based on presence or absence of orthologs of each of a plurality of query genes that are co-localized with the BGC in a plurality of different genomes, wherein the plurality of genomes comprises a plurality of positive genomes comprising an ortholog of an anchor gene of the BGC, and a plurality of negative genomes that do not comprise an ortholog of the anchor gene of the BGC, wherein the anchor gene is known to associate with the BGC;
ii) one or more phylogenetic features of the last common ancestor (LCA) of homologs of the pETaG in a phylogenetic tree of the plurality of genomes;
iii) one or more scores indicative of co-occurrence of the ortholog of the pETaG and the ortholog of the anchor gene among the plurality of positive genomes;
iv) one or more scores indicative of co-evolution of sequence divergence among orthologs of the pETaG with respect to sequence divergence among orthologs of the anchor gene in positive genomes comprising both an ortholog of the pETaG and an ortholog of the anchor gene; and
v) one or more scores indicative of copy number of homologs of the pETaG in the plurality of positive genomes and copy number of homologs of the pETaG in the plurality of negative genomes; and
b) determining the likelihood that the pETaG is a resistance gene against the secondary metabolite produced by the BGC based on the one or more parameters.
   98. A system comprising:
one or more processors; and
   a memory communicatively coupled to the one or more processors and configured to store instructions that, when executed by the one or more processors, cause the system to perform the method of any one of embodiments 1 to 95.
   99. A non-transitory computer-readable storage medium storing one or more programs, the one or more programs comprising instructions, which when executed by one or more processors of an electronic device, cause the electronic device to perform any of the methods of embodiments 1 to 95.

The foregoing description, for the purpose of explanation, has been described with reference to specific examples or aspects. However, the illustrative discussions above are not intended to be exhaustive or to limit the invention to the precise forms disclosed. For the purpose of clarity and a concise description, features are described herein as part of the same or separate variations; however, it will be appreciated that the scope of the disclosure includes variations having combinations of all or some of the features described. Many modifications and variations are possible in view of the above teachings. The variations were chosen and described in order to best explain the principles of the techniques and their practical applications. Others skilled in the art are thereby enabled to best utilize the techniques and various variations with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1-44. (canceled)

45. A computer-implemented method of determining a likelihood that a putative embedded target gene (pETaG) is a resistance gene against a secondary metabolite produced by a biosynthetic gene cluster (BGC) in a query genome, the method comprising:
a) determining one or more parameters selected from the following:
i) a likelihood that the pETaG is associated with the BGC based on presence or absence of orthologs of each of a plurality of query genes that are co-localized with the BGC in a plurality of different genomes, wherein the plurality of genomes comprises a plurality of positive genomes comprising an ortholog of an anchor gene of the BGC, and a plurality of negative genomes that do not comprise an ortholog of the anchor gene of the BGC, wherein the anchor gene is known to associate with the BGC;
ii) one or more phylogenetic features of the last common ancestor (LCA) of homologs of the pETaG in a phylogenetic tree of the plurality of genomes;
iii) one or more scores indicative of co-occurrence of the ortholog of the pETaG and the ortholog of the anchor gene among the plurality of positive genomes;
iv) one or more scores indicative of co-evolution of sequence divergence among orthologs of the pETaG with respect to sequence divergence among orthologs of the anchor gene in positive genomes comprising both an ortholog of the pETaG and an ortholog of the anchor gene; and
v) one or more scores indicative of copy number of homologs of the pETaG in the plurality of positive genomes and copy number of homologs of the pETaG in the plurality of negative genomes; and
b) determining the likelihood that the pETaG is a resistance gene against the secondary metabolite produced by the BGC based on the one or more parameters.

46. The computer-implemented method of claim 45, wherein the pETaG is co-localized with the BGC in the query genome.

47. The computer-implemented method of claim 46, wherein the pETaG is not involved in production of the secondary metabolite by the BGC.

48. The computer-implemented method of claim 45, wherein the anchor gene is the core synthase gene of the BGC.

49. The computer-implemented method of claim 45, wherein the method comprises determining a likelihood for each of a plurality of pETaGs that the pETaG is a resistance gene against a secondary metabolite produced by a BGC in a target genome.

50. The computer-implemented method of claim 49, comprising:
a) identifying putative BGCs in a plurality of genomes having pairwise sequence similarity above a threshold value;
b) identifying non-biosynthetic genes that are co-localized with orthologs of the anchor gene in the putative BGCs, wherein the non-biosynthetic genes are homologous to any one of a plurality of query genes in an organism of interest, and wherein the non-biosynthetic genes are not involved in production of secondary metabolites by the BGCs;
c) for each of the plurality of query genes, identifying the non-biosynthetic gene encoding a protein having the highest sequence similarity to the protein of the respective target gene as the pETaG and the genome encoding the non-biosynthetic gene as the target genome; and
d) for each of the plurality of query genes, determining a likelihood that the respective pETaG is a resistance gene against a secondary metabolite produced by the respective BGC in the respective target genome.

51. The computer-implemented method of claim 49, comprising:
a) clustering genomes in a database into a plurality of clusters each comprising genomes having pairwise sequence similarity above a threshold value;
b) for each of the plurality of clusters:
i) identifying non-biosynthetic genes that are co-localized with orthologs of the anchor gene in the putative BGCs, wherein the non-biosynthetic genes are homologous to any one of a plurality of query genes in an organism of interest, and wherein the non-biosynthetic genes are not involved in production of secondary metabolites by the BGCs;
ii) for each of the plurality of query genes, identifying the non-biosynthetic gene encoding a protein having the highest sequence similarity to the protein of the respective query gene as a candidate pETaG; and
c) clustering the candidate pETaGs into a plurality of clusters based on sequence similarities among the pETaGs, and identifying the candidate pETaG encoding a protein having the highest sequence similarity to the protein of the respective query gene in each cluster as the pETaG and the respective genome encoding the pETaG as the target genome; and
d) for each of the plurality of query genes, determining a likelihood that the respective pETaG is a resistance gene against a secondary metabolite produced by the respective BGC in the respective target genome.

52. The computer-implemented method of claim 50, wherein the threshold value is at least 70%, at least 75%, at least 80%, at least 85%, at least 90%, at least 95%, or at least 98% pairwise sequence similarity.

53. The computer-implemented method of claim 50, wherein each of the identified non-biosynthetic genes encodes a protein having at least about 30% sequence identity to a protein encoded by the respective query gene.

54. The computer-implemented method of claim 50, wherein the plurality of query genes are all protein-coding genes in the organism of interest.

55. The computer-implemented method of claim 50, wherein the organism of interest is a mammal.

56. The computer-implemented method of claim 55, wherein the organism of interest is human.

57. The computer-implemented method of claim 50, wherein the organism of interest is a reptile, a bird, an amphibian, a plant, a fungus, or a bacterium.

58. The computer-implemented method of claim 56, wherein the plurality of genomes are fungal genomes, bacterial genomes, or plant genomes.

59-60. (canceled)

61. The computer-implemented method of claim 54, wherein each of the plurality of clusters comprise about 10 to about 100 genomes.

62. A computer-implemented method of identifying a druggable target in an organism of interest, comprising performing the method of claim 45, and identifying a query gene as a druggable target based on the likelihood that the respective pETaG of the query gene is a resistance gene against a secondary metabolite produced by the BGC in the target genome.

63. The computer-implemented method of claim 62, further comprising identifying the secondary metabolite or an analog thereof as a small molecule modulator of the query gene or a protein encoded by the query gene.

64. The computer-implemented method of claim 63, further comprising contacting the secondary metabolite or analog thereof with a protein encoded by the query gene, and detecting an activity of the protein encoded by the query gene.

65. The computer-implemented method of claim 45, wherein the number of the positive genomes is equal to the number of the negative genomes.

66. The computer-implemented method of claim 65, comprising selecting a plurality of positive genomes and a plurality of negative genomes from a database of genomes.

67. The computer-implemented method of claim 66, comprising clustering the database of genomes into a plurality of clusters based on sequence similarities, and selecting one positive genome per cluster to provide the plurality of positive genomes.

68. The computer-implemented method of claim 67, comprising selecting a negative genome that has highest sequence similarity to each positive genome in the cluster.

69. The computer-implemented method of claim 66, wherein the average pairwise percentage sequence identities of orthologs of one or more single copy genes in the positive genomes is no more than about 95%, and/or the average pairwise percentage sequence identities of orthologs of one or more single copy genes in the negative genomes is no more than about 95%.

70. The computer-implemented method of claim 45, wherein the number of the positive genomes is at least 5.

71. The computer-implemented method of claim 45, wherein the one or more parameters comprises a likelihood that the pETaG is associated with the BGC based on presence or absence of orthologs of each of a plurality of query genes in the BGC in a plurality of different genomes.

72-78. (canceled)

79. The computer-implemented method of claim 45, wherein the one or more parameters comprises one or more phylogenetic features of the last common ancestor (LCA) of homologs of the pETaG in a phylogenetic tree of the plurality of positive and negative genomes.

80. The computer-implemented method of claim 79, wherein the one or more phylogenetic features are selected from the group consisting of average copy number difference (CND) between genes in the plurality of positive genomes and genes in the plurality of negative genomes and values determined from a plurality of positive genomes, ratio mean to LCA, ratio standard deviation to LCA, mean ratio of neighbor distance, standard deviation of ratio of neighbor distance, and sum of ratio clade.

81. The computer-implemented method of claim 45, wherein the one or more parameters comprise one or more scores indicative of co-occurrence of the ortholog of the pETaG and the ortholog of the anchor gene among the plurality of positive genomes.

82. The computer-implemented method of claim 81, wherein the one or more scores indicative of co-occurrence are selected from the group consisting of co-occurrence pETaG distance, co-occurrence pETaG rank, co-occurrence core distance, and co-occurrence core rank.

83. The computer-implemented method of claim 45, wherein the one or more parameters comprise one or more scores indicative of co-evolution of sequence divergence among orthologs of the pETaG with respect to sequence divergence among orthologs of the anchor gene in positive genomes comprising both an ortholog of the pETaG and an ortholog of the anchor gene.

84. The computer-implemented method of claim 82, wherein the one or more scores indicative of co-evolution are selected from the group consisting of co-evolution correlation, co-evolution rank, and co-evolution slope.

85. The method of claim 79, wherein the one or more parameters further comprise one or more features of the plurality of positive genomes and the plurality of negative genomes.

86. The computer-implemented method of claim 85, wherein the one or more features are selected from the group consisting of the number of the positive genomes, mean pairwise genomic identity (PGI) among the positive genomes, standard deviation of PGI among the positive genomes, the number of the negative genomes, mean PGI among the negative genomes, and standard deviation of PGI among the negative genomes.

87. The computer-implemented method of claim 45, wherein said determining the likelihood based on the one or more parameters comprises inputting the one or more features into a machine-learning model, wherein the machine-learning model has been trained to determine a likelihood that the pETaG is a resistance gene.

88. The computer-implemented method of claim 87, wherein the machine-learning model is a deep learning model.

89. The computer-implemented method of claim 87, wherein the machine-learning model is a decision tree model.

90. The computer-implemented method of claim 87, wherein the machine-learning model is a Bayesian inference model.

91. The computer-implemented method of claim 87, wherein the machine-learning model is a logistic regression model.

92. The computer-implemented method of claim 45, wherein whether a gene is co-localized with an anchor gene of a BGC is determined using antiSMASH.

93. The computer-implemented method of claim 45, wherein whether a gene is co-localized with an anchor gene of a BGC is determined based on whether the gene is located within a proximity distance from the anchor gene.

94. The computer-implemented method of claim 93, wherein the proximity zone is no more than about 50 kb.

95. The computer-implemented method of claim 93, wherein the proximity zone is about 20 kb.

96. (canceled)

97. A system comprising:
one or more processors; and
a memory communicatively coupled to the one or more processors and configured to store instructions that, when executed by the one or more processors, cause the system to perform a method of determining a likelihood that a putative embedded target gene (pETaG) is a resistance gene against a secondary metabolite produced by a biosynthetic gene cluster (BGC) in a query genome, the method comprising:
a) determining one or more parameters selected from the following:
i) a likelihood that the pETaG is associated with the BGC based on presence or absence of orthologs of each of a plurality of query genes that are co-localized with the BGC in a plurality of different genomes, wherein the plurality of genomes comprises a plurality of positive genomes comprising an ortholog of an anchor gene of the BGC, and a plurality of negative genomes that do not comprise an ortholog of the anchor gene of the BGC, wherein the anchor gene is known to associate with the BGC;
ii) one or more phylogenetic features of the last common ancestor (LCA) of homologs of the pETaG in a phylogenetic tree of the plurality of genomes;
iii) one or more scores indicative of co-occurrence of the ortholog of the pETaG and the ortholog of the anchor gene among the plurality of positive genomes;
iv) one or more scores indicative of co-evolution of sequence divergence among orthologs of the pETaG with respect to sequence divergence among orthologs of the anchor gene in positive genomes comprising both an ortholog of the pETaG and an ortholog of the anchor gene; and
v) one or more scores indicative of copy number of homologs of the pETaG in the plurality of positive genomes and copy number of homologs of the pETaG in the plurality of negative genomes; and
b) determining the likelihood that the pETaG is a resistance gene against the secondary metabolite produced by the BGC based on the one or more parameters.

98. A system comprising:
one or more processors; and
a memory communicatively coupled to the one or more processors and configured to store instructions that, when executed by the one or more processors, cause the system to perform the method of claim 45.

99. A non-transitory computer-readable storage medium storing one or more programs, the one or more programs comprising instructions, which when executed by one or more processors of an electronic device, cause the electronic device to perform any of the methods of claim 45.
